(19) United States
(12) Patent Application Publication
(10) Pub. No.: US 2024/0209408 A1
(43) Pub. Date: Jun. 27, 2024

(54) ENGINEERED 3-O-KINASE VARIANTS AND METHODS OF USE

(71) Applicant: Codexis, Inc., Redwood City, CA (US)

(72) Inventors: David Entwistle, San Carlos, CA (US); Stephanie Marie Forgel, Redwood City, CA (US); Anders Matthew Knight, Mountain View, CA (US); Mikayla Krawczyk, Redwood City, CA (US); Philip Provencher, San Francisco, CA (US); Amani Shoubber, Redwood City, CA (US); Jonathan Vroom, South San Francisco, CA (US); David Watts, San Mateo, CA (US); Leland Wong, San Bruno, CA (US)

(73) Assignee: Codexis, Inc., Redwood City, CA (US)

(21) Appl. No.: 18/485,925

(22) Filed: Oct. 12, 2023

Related U.S. Application Data
(60) Provisional application No. 63/387,908, filed on Dec. 16, 2022.

(51) Int. Cl. C12P 19/38; C12N 9/02; C12N 9/12

(52) U.S. Cl. CPC C12P 19/38; C12N 9/0008; C12N 9/1205; C12Y 102/03003; C12Y 207/01

(57) ABSTRACT

The present invention provides engineered 3′O-kinase polypeptides useful for construction of materials used in template-independent polynucleotide synthesis, as well as compositions and methods of utilizing these engineered polypeptides. The present disclosure also describes one-pot methods for conversion of a natural or modified nucleoside to a nucleoside tetraphosphate or NQP.

### CROSS REFERENCE TO RELATED APPLICATIONS

This application claims the priority benefit of U.S. Provisional Patent Application No. 63/387,908, filed Dec. 16, 2023, the entirety of which is incorporated herein by reference.

### TECHNICAL FIELD

The present invention provides engineered 3′O-kinase polypeptides useful for construction of materials used in template-independent polynucleotide synthesis, as well as compositions and methods of utilizing these engineered polypeptides. The present disclosure also describes one-pot methods for conversion of a natural or modified nucleoside to a nucleoside tetraphosphate or NQP.

### REFERENCE TO SEQUENCE LISTING, TABLE OR COMPUTER PROGRAM

The official copy of the Sequence Listing is submitted concurrently with the specification as an XML file, with a file name of “CX10-241WO2_ST26.xml”, a creation date of Oct. 11, 2023, and a size of 3,729,984 bytes. The Sequence Listing filed is part of the specification and is incorporated in its entirety by reference herein.

### BACKGROUND

Synthetic biology is becoming established in a diverse range of high value, high growth markets. From food and agriculture to therapeutics, diagnostics, and vaccines; tools such as gene editing, DNA sequencing and gene synthesis are being used to build value-added products with advanced functionality (e.g., cell bioreactors, etc.) and desired end products (e.g., drugs, chemicals, etc.). The barrier to widespread implementation of these technologies is the ability to efficiently synthesize RNA, DNA, and other polynucleotides.

In particular, silencing RNA (siRNA) therapeutics are, amongst other polynucleotides, a promising class of drugs that have the potential to treat numerous difficult to treat conditions in a highly targeted manner by binding to known mRNA targets (Hu et al. (2020). Sig Transduct Target Ther 5, 101; Zhang et al. (2021). Bioch. Pharmac., 189, 114432.) As these therapies become more common and are targeted at larger patient populations, the ability to produce large amounts of the oligonucleotide active pharmaceutical ingredient (API) becomes critical.

Phosphoramidite chemistry has been developed extensively over the years to synthesize small amounts of DNA and RNA, but suffers from several cost, processing and sustainability issues that are potentially limiting as API demand grows to triple-quadruple digit kilograms per year (Andrews et al. (2021). J. Org. Chem. 86, 49-61). Additionally, RNA synthesis using phosphoramidite synthesis chemistry is limited to producing short oligonucleotides of approximately 200 basepairs (Beaucage & Caruthers. (1981). Tetrahedron Lett. 22 (20): 1859.)

New oligonucleotide synthesis techniques are being developed to replace to phosphoramidite chemistry to meet the growing demand for large quantities of DNA and RNA necessary for modern medical and industrial applications. The most promising of these is template independent oligonucleotide synthesis using various polymerases, including terminal nucleotide transferases (TdTs) and polyX polymerases. These methods often rely on modified nucleotide triphosphates (NTPs) that incorporate blocking groups or other structural or chemical elements that allow the controlled addition of a defined sequence of NTPs. These modified NTPs include NTPs with blocking groups on the 3′ or 2′ positions of the sugar, as well as NTPs with modified bases or thiol derivates for the formation of more stable oligonucleotide phosphorthioate backbone bonds.

As these methods mature, a limiting factor is the commercial availability and cost of natural and modified NTPs for synthesis reactions. In particular, NTPs with a phosphate at the 3′ position of the sugar (nucleoside tetraphosphates, pppNps or NQPs), with or without additional modifications to the nucleobase, sugar, and/or phosphate chain, are useful for emerging template independent synthesis applications. However, these NQPs are not widely commercially available and are cost prohibitive at an industrial scale. In conclusion, new methods to synthesize natural and modified NTPs and NQPs are necessary to enable production of oligonucleotides on the scale required for modern synthetic biology applications.

### SUMMARY

The present disclosure provides engineered 3′O-kinase polypeptides useful for the synthesis of nucleoside tetraphosphates (pppNps or NQPs), as well as compositions and methods of utilizing these engineered polypeptides. The 3′O-kinases of the present disclosure are variants of the wild-type dephospho-CoA kinase (CoaE) gene from Geobacillus stearothermophilus (SEQ ID NO: 10). These engineered 3′O-kinases are capable of adding a phosphate group to the 3′ position of the sugar of a natural or modified NTP to produce an NQP. The present disclosure provides various methods of synthesizing natural and modified NQPs.

In some embodiments, the present disclosure provides an engineered 3′O-kinase polypeptide comprising an amino acid sequence having at least 80%, 85%, 90%, 91%, 92%, 93%, 94%, 95%, 96%, 97%, 98%, 99%, or more sequence identity to a reference sequence selected from SEQ ID NO: 10, 142, 372, 450, 496, 1042, 1180, and 1412, comprising at least one substitution or one substitution set at one or more positions, wherein the positions are numbered with reference to SEQ ID NO: 10 and wherein the engineered 3′O-kinase polypeptide has increased activity on natural substrates, increased activity on non-natural substrates, increased selectivity, increased substrate promiscuity, decreased product inhibition and/or decreased byproduct formation, as compared to a wild-type or reference 3′O-kinase known to those of skill in the art. These engineered 3′O-kinase polypeptides with one or more amino acid substitutions or substitution sets are described, below, in the detailed description of the invention.

In some additional embodiments, the engineered polypeptide comprises an amino acid sequence with at least 60%, 80%, 85%, 90%, 91%, 92%, 93%, 94%, 95%, 96%, 97%, 98%, 99%, or more sequence identity to any even-numbered sequence selected from SEQ ID NO: 56-366, or 372-2122.

The present disclosure also provides an engineered polynucleotide encoding at least one engineered polypeptide described in the above paragraphs. In some embodiments, the engineered polynucleotide comprises the odd-numbered sequences selected from SEQ ID NO: 55-365, or 371-2121.

The present disclosure further provides vectors comprising at least one engineered polynucleotide described above. In some embodiments, the vectors further comprise at least one control sequence.

The present disclosure also provides host cells comprising the vectors provided herein. In some embodiments, the host cell produces at least one engineered polypeptide provided herein.

The present disclosure further provides methods of producing an engineered 3′O-kinase polypeptide, comprising the steps of culturing the host cell provided herein under conditions such that the engineered polynucleotide is expressed and the engineered polypeptide is produced. In some embodiments, the methods further comprise the step of recovering the engineered polypeptide.

The present disclosure further provides a one-pot method for conversion of nucleosides to NQPs comprising a 5′O-kinase enzyme, a nucleoside diphosphate kinase enzyme, an acetate kinase enzyme, and a 3′O-kinase enzyme, under suitable reaction conditions for conversion of a natural or modified nucleoside to a natural or modified NQP, optionally including an acetate kinase enzyme and/or a pyruvate oxidase enzyme and/or other suitable recycling enzymes. In some embodiments, the one-pot method is a telescoping method, comprising two steps.

### BRIEF DESCRIPTION OF THE DRAWINGS

FIG. 1 depicts Scheme 3—a one-pot method for conversion of nucleosides to NQPs.

FIG. 2 depicts Scheme 4—the first step conversion of a nucleoside to an NTP in a one-pot method for conversion of nucleosides to NQPs that occurs in two steps.

### DESCRIPTION OF THE INVENTION

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the invention as a whole.

It is to be understood that both the foregoing general description and the following detailed description are exemplary and explanatory only and are not restrictive of the present invention. The section headings used herein are for organizational purposes only and not to be construed as limiting the subject matter described. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used in this specification and the appended claims, the singular forms “a”, “an” and “the” include plural referents unless the context clearly indicates otherwise. Thus, for example, reference to “a polypeptide” includes more than one polypeptide. Similarly, “comprise,” “comprises,” “comprising” “include,” “includes,” and “including” are interchangeable and not intended to be limiting.

It is to be understood that where descriptions of various embodiments use the term “comprising,” those skilled in the art would understand that in some specific instances, an embodiment can be alternatively described using language “consisting essentially of” or “consisting of.” It is to be further understood that where descriptions of various embodiments use the term “optional” or “optionally” the subsequently described event or circumstance may or may not occur, and that the description includes instances where the event or circumstance occurs and instances in which it does not. It is to be understood that both the foregoing general description, and the following detailed description are exemplary and explanatory only and are not restrictive of this disclosure. The section headings used herein are for organizational purposes only and not to be construed as limiting the subject matter described.

### Abbreviations

The abbreviations used for the genetically encoded amino acids are conventional and are as follows:

| Amino Acid | Three-Letter | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | HIS | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). These abbreviations are also used interchangeably for nucleosides and nucleotides (nucleosides with one or more phosphate groups). Unless specifically delineated, the abbreviated nucleosides or nucleotides may be either ribonucleosides (or ribonucleotides) or 2′-deoxyribonucleosides (or 2′-deoxyribonucleotides). The nucleosides or nucleotides may also be modified at the 3′ position. The nucleosides or nucleotides may be specified as being either ribonucleosides (or ribonucleotides) or 2′-deoxyribonucleosides (or 2′-deoxyribonucleotides) on an individual basis or on an aggregate basis. When nucleic acid sequences are presented as a string of one-letter abbreviations, the sequences are presented in the 5′ to 3′ direction in accordance with common convention, and the phosphates are not indicated.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

As used herein, “polynucleotide,” “oligonucleotide,” and “nucleic acid” are used interchangeably herein and refer to two or more nucleosides or nucleotides that are covalently linked together. The polynucleotide may be wholly comprised of ribonucleotides (i.e., RNA), wholly comprised of 2′deoxyribonucleotides (i.e., DNA), wholly comprised of other synthetic nucleotides or comprised of mixtures of synthetic, ribo- and/or 2′ deoxyribonucleotides. The polynucleotides may also include modified nucleotides with substitutions, including 2′ substitutions (e.g., 2′-flouro, 2′-O-methyl, 2′-O-methoxyethyl, locked or constrained ethyl modifications, and others known to those skilled in the art). Nucleosides will be linked together via standard phosphodiester linkages or via one or more non-standard linkages, including but not limited to phosphothiolated linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino-acid sequences. Nucleobases that are modified or synthetic may comprise any known or hypothetical or future discovered modification or structure that would be recognized by one of skill in the art as a modified or synthetic nucleobase. Similarly, the terms “polynucleotide,” “oligonucleotide,” and “nucleic acid” are intended to comprise any modified or synthetic structure that is now known or discovered in the future that would be recognized by one of skill in the art as being or having the function of a “polynucleotide,” “oligonucleotide,” or “nucleic acid.” An example of a modified or synthetic structure having the function of a “polynucleotide,” “oligonucleotide,” or “nucleic acid” is PNA or peptide nucleic acid.

As used herein, “template-independent synthesis” refers to synthesis of an oligonucleotide or a polynucleotide without the use of template strand as a guide for synthesis of a complementary oligo or polynucleotide strand. Thus, template-independent synthesis refers to an iterative process, whereby, successive NTPs are added to a growing oligo or nucleotide chain or acceptor substrate. Template-independent synthesis may be in a sequence defined manner or may be random, as is the case with the wild-type TdT in creating antigen receptor diversity. Processes for template-independent synthesis are further described herein.

“Coding sequence” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “recombinant,” “engineered,” and “non-naturally occurring” when used with reference to a cell, nucleic acid, or polypeptide, refer to a material, or a material corresponding to the natural or native form of the material, that has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity” and “percentage homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970]), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 215: 403-410 [1990]; and Altschul et al., Nucl. Acids Res., 3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, a “reference sequence based on SEQ ID NO:4 having at the residue corresponding to X14 a valine” or X14V refers to a reference sequence in which the corresponding residue at X14 in SEQ ID NO:4, which is a tyrosine, has been changed to valine.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “substantial identity” refers to a polynucleotide or polypeptide sequence that has at least 80 percent sequence identity, at least 85 percent identity, at least between 89 to 95 percent sequence identity, or more usually, at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). In some embodiments, residue positions that are not identical in sequences being compared differ by conservative amino acid substitutions.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered 3′O-kinase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” or “residue difference” refers to a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X25 as compared to SEQ ID NO: 2” refers to a change of the amino acid residue at the polypeptide position corresponding to position 25 of SEQ ID NO:2. Thus, if the reference polypeptide of SEQ ID NO: 2 has a valine at position 25, then a “residue difference at position X25 as compared to SEQ ID NO:2” an amino acid substitution of any residue other than valine at the position of the polypeptide corresponding to position 25 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some embodiments, more than one amino acid can appear in a specified residue position (i.e., the alternative amino acids can be listed in the form XnY/Z, where Y and Z represent alternate amino acid residues). In some instances (e.g., in Tables 13.1, 13.2, 13.3, 13.4, and 13.5.) the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present invention can include one or more amino acid residue differences relative to a reference sequence, which is indicated by a list of the specified positions where changes are made relative to the reference sequence. In some additional embodiments, the present invention provides engineered polypeptide sequences comprising both conservative and non-conservative amino acid substitutions.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain is substituted with another aliphatic amino acid (e.g., alanine, valine, leucine, and isoleucine); an amino acid with an hydroxyl side chain is substituted with another amino acid with a hydroxyl side chain (e.g., serine and threonine); an amino acid having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table 1 below.

TABLE 1
Conservative Amino Acid Substitution Examples

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered 3′O-kinase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered 3′O-kinase enzymes comprise insertions of one or more amino acids to the naturally occurring polypeptide as well as insertions of one or more amino acids to other improved 3′O-kinase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Fragment” as used herein refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99% of the full-length 3′O-kinase polypeptide, for example the polypeptide of SEQ ID NO: 2 or an 3′O-kinase provided in the even-numbered sequences of SEQ ID NOs: 4-1960.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The engineered 3′O-kinase enzymes may be present within a cell, present in the cellular medium, or prepared in various forms, such as lysates or isolated preparations. As such, in some embodiments, the engineered 3′O-kinase enzyme can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure 3′O-kinase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered 3′O-kinase polypeptide is a substantially pure polypeptide composition.

As used herein, “improved enzyme property” refers to at least one improved property of an enzyme. In some embodiments, the present invention provides engineered 3′O-kinase polypeptides that exhibit an improvement in any enzyme property as compared to a reference 3′O-kinase polypeptide and/or a wild-type 3′O-kinase polypeptide, and/or another engineered 3′O-kinase polypeptide. For the engineered 3′O-kinase polypeptides described herein, the comparison is generally made to the wild-type enzyme from which the 3′O-kinase is derived, although in some embodiments, the reference enzyme can be another improved engineered 3′O-kinase. Thus, the level of “improvement” can be determined and compared between various 3′O-kinase polypeptides, including wild-type, as well as engineered 3′O-kinases. Improved properties include, but are not limited, to such properties as enzymatic activity (which can be expressed in terms of percent conversion of the substrate), thermo stability, solvent stability, pH activity profile, cofactor requirements, refractoriness to inhibitors (e.g., substrate or product inhibition), activity at elevated temperatures, increased soluble expression, decreased by-product formation, increased specific activity substrates, and/or increased activity (including enantioselectivity).

“Increased enzymatic activity” refers to an improved property of the 3′O-kinase polypeptides, which can be represented by an increase in specific activity (e.g., product produced/time/weight protein) or an increase in percent conversion of the substrate to the product (e.g., percent conversion of starting amount of substrate to product in a specified time period using a specified amount of 3′O-kinase) as compared to the reference 3′O-kinase enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.2 times the enzymatic activity of the corresponding wild-type enzyme, to as much as 2 times, 5 times, 10 times, 20 times, 25 times, 50 times or more enzymatic activity than the naturally occurring or another engineered 3′O-kinase from which the 3′O-kinase polypeptides were derived. 3′O-kinase activity can be measured by any one of standard assays, such as by monitoring changes in properties of substrates, cofactors, or products. In some embodiments, the amount of products generated can be measured by Liquid Chromatography-Mass Spectrometry (LC-MS), HPLC, or other methods, as known in the art. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Conversion” refers to the enzymatic conversion of the substrate(s) to the corresponding product(s). “Percent conversion” refers to the percent of the substrate that is converted to the product within a period of time under specified conditions. Thus, the “enzymatic activity” or “activity” of a 3′O-kinase polypeptide can be expressed as “percent conversion” of the substrate to the product.

“Thermostable” refers to a 3′O-kinase polypeptide that maintains similar activity (more than 60% to 80% for example) after exposure to elevated temperatures (e.g., 40-80° C.) for a period of time (e.g., 0.5-24 hrs) compared to the wild-type enzyme exposed to the same elevated temperature.

“Solvent stable” refers to a 3′O-kinase polypeptide that maintains similar activity (more than e.g., 60% to 80%) after exposure to varying concentrations (e.g., 5-99%) of solvent (ethanol, isopropyl alcohol, dimethylsulfoxide (DMSO), tetrahydrofuran, 2-methyltetrahydrofuran, acetone, toluene, butyl acetate, methyl tert-butyl ether, etc.) for a period of time (e.g., 0.5-24 hrs) compared to the wild-type enzyme exposed to the same concentration of the same solvent.

“Thermo- and solvent stable” refers to a 3′O-kinase polypeptide that is both thermostable and solvent stable.

The term “stringent hybridization conditions” is used herein to refer to conditions under which nucleic acid hybrids are stable. As known to those of skill in the art, the stability of hybrids is reflected in the melting temperature (Tₘ) of the hybrids. In general, the stability of a hybrid is a function of ion strength, temperature, G/C content, and the presence of chaotropic agents. The Tₘ values for polynucleotides can be calculated using known methods for predicting melting temperatures (See e.g., Baldino et al., Meth. Enzymol., 168:761-777 [1989]; Bolton et al., Proc. Natl. Acad. Sci. USA 48:1390 [1962]; Bresslauer et al., Proc. Natl. Acad. Sci. USA 83:8893-8897 [1986]; Freier et al., Proc. Natl. Acad. Sci. USA 83:9373-9377 [1986]; Kierzek et al., Biochem., 25:7840-7846 [1986]; Rychlik et al., 1990, Nucl. Acids Res., 18:6409-6412 [1990] (erratum, Nucl. Acids Res., 19:698 [1991]); Sambrook et al., supra); Suggs et al., 1981, in Developmental Biology Using Purified Genes, Brown et al. [eds.], pp. 683-693, Academic Press, Cambridge, MA [1981]; and Wetmur, Crit. Rev. Biochem. Mol. Biol., 26:227-259 [1991]). In some embodiments, the polynucleotide encodes the polypeptide disclosed herein and hybridizes under defined conditions, such as moderately stringent or highly stringent conditions, to the complement of a sequence encoding an engineered 3′O-kinase enzyme of the present invention.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5× Denhart's solution, 5× SSPE, 0.2% SDS at 42° C., followed by washing in 0.2× SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5× Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the 3′O-kinase enzymes may be codon optimized for optimal production from the host organism selected for expression.

As used herein, “preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See e.g., GCG CodonPreference, Genetics Computer Group Wisconsin Package; CodonW, Peden, University of Nottingham; McInerney, Bioinform., 14:372-73 [1998]; Stenico et al., Nucl. Acids Res., 222437-46 [1994]; Wright, Gene 87:23-29 [1990]). Codon usage tables are available for many different organisms (See e.g., Wada et al., Nucl. Acids Res., 20:2111-2118 [1992]; Nakamura et al., Nucl. Acids Res., 28:292 [2000]; Duret, et al., supra; Henaut and Danchin, in Escherichia coli and Salmonella, Neidhardt, et al. (eds.), ASM Press, Washington D.C., p. 2047-2066 [1996]). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (See e.g., Mount, Bioinformatics: Sequence and Genome Analysis, Chapter 8, Cold Spring Harbor Laboratory Press, Cold Spring Harbor, N.Y. [2001]; Uberbacher, Meth. Enzymol., 266:259-281 [1996]; and Tiwari et al., Comput. Appl. Biosci., 13:263-270 [1997]).

“Control sequence” is defined herein to include all components, which are necessary or advantageous for the expression of a polynucleotide and/or polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refer to those conditions in the biocatalytic reaction solution (e.g., ranges of enzyme loading, substrate loading, cofactor loading, temperature, pH, buffers, co-solvents, etc.) under which a 3′O-kinase polypeptide of the present invention is capable of converting one or more substrate compounds to a product compound. Exemplary “suitable reaction conditions” are provided in the present invention and illustrated by the Examples.

“Composition” refers to a mixture or combination of one or more substances, wherein each substance or component of the composition retains its individual properties. As used herein, a biocatalytic composition refers to a combination of one or more substances useful for biocatalysis.

“Loading”, such as in “compound loading” or “enzyme loading” or “cofactor loading” refers to the concentration or amount of a component in a reaction mixture at the start of the reaction.

“Substrate” in the context of a biocatalyst mediated process refers to the compound or molecule acted on by the biocatalyst. For example, a 3′O-kinase biocatalyst used in the synthesis processes disclosed herein acts on a natural or modified NTP.

“Product” in the context of a biocatalyst mediated process refers to the compound or molecule resulting from the action of the biocatalyst. For example, an exemplary product for a 3′O-kinase biocatalyst used in a process disclosed herein is an NQP.

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁-C₆)alkyl refers to an alkyl of 1 to 6 carbon atoms).

“Alkenyl” refers to hydrocarbon groups of from 2 to 12 carbon atoms inclusively, either straight or branched containing at least one double bond but optionally containing more than one double bond.

“Alkynyl” refers to hydrocarbon groups of from 2 to 12 carbon atoms inclusively, either straight or branched containing at least one triple bond but optionally containing more than one triple bond, and additionally optionally containing one or more double bonded moieties.

“Heteroalkyl, “heteroalkenyl,” and heteroalkynyl,” refer respectively, to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to-O—, —S—, —S—O—, —NR^{γ}—, —PH—, —S(O)—, —S(O)2-, —S(O) NR^{γ}—, —S(O)₂NR^{γ}, and the like, including combinations thereof, where each Rr is independently selected from hydrogen, alkyl, cycloalkyl, heterocycloalkyl, aryl, and heteroaryl.

“Amino” refers to the group —NH₂. Substituted amino refers to the group —NHR^{η}, NR^{η}Rη, and NR^{η}R^{η}R^{η}, where each R^{η} is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with one or more amino groups, including substituted amino groups.

“Aminocarbonyl” refers to —C(O)NH₂. Substituted aminocarbonyl refers to —C(O)NRηR^{η}, where the amino group NR^{η}R^{η} is as defined herein.

“Oxy” refers to a divalent group —O—, which may have various substituents to form different oxy groups, including ethers and esters.

“Alkoxy” or “alkyloxy” are used interchangeably herein to refer to the group —OR, wherein R is an alkyl group, including optionally substituted alkyl groups.

“Carboxy” refers to —COOH.

“Carbonyl” refers to —C(O)—, which may have a variety of substituents to form different carbonyl groups including acids, acid halides, aldehydes, amides, esters, and ketones.

“Carboxyalkyl” refers to an alkyl in which one or more of the hydrogen atoms are replaced with one or more carboxy groups.

“Aminocarbonylalkyl” refers to an alkyl substituted with an aminocarbonyl group, as defined herein.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl group in which in which one or more of the hydrogen atoms are replaced with one or more hydroxy groups.

“Thiol” or “sulfanyl” refers to —SH. Substituted thiol or sulfanyl refers to —S—R^{η}, where R^{η} is an alkyl, aryl or other suitable substituent.

“Sulfonyl” refers to —SO₂—. Substituted sulfonyl refers to —SO₂—R^{η}, where R^{η} is an alkyl, aryl or other suitable substituent.

“Alkylsulfonyl” refers to —SO₂—R^{ζ}, where R^{ζ} is an alkyl, which can be optionally substituted. Typical alkylsulfonyl groups include, but are not limited to, methylsulfonyl, ethylsulfonyl, n-propylsulfonyl, and the like.

“Phosphate” as used herein refers to a functional group comprised of an orthophosphate ion (phosphorous atom covalently linked to four oxygen atoms). The orthophosphate ion is commonly found with one or more hydrogen atoms or organic groups. A phosphate group or chain may be modified, as further described herein.

“Phosphorylated” as used herein refers to the addition or presence of one of more phosphoryl groups (phosphorous atom covalently linked to the three oxygen atoms).

“thiophosphate” refers to an instance where a non-bridging oxygen in a phosphate group of a phosphodiester bond, NMP, NDP, NTP or NQP is replaced with a sulfur.

“dithiophosphate” refers to an instance where two non-bridging oxygens in a phosphate group of a phosphodiester bond, NMP, NDP, NTP or NQP are replaced with two sulfurs

“Optionally substituted” as used herein with respect to the foregoing chemical groups means that positions of the chemical group occupied by hydrogen can be substituted with another atom (unless otherwise specified) exemplified by, but not limited to carbon, oxygen, nitrogen, or sulfur, or a chemical group, exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; where preferred heteroatoms are oxygen, nitrogen, and sulfur. Additionally, where open valences exist on these substitute chemical groups they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further contemplated that the above substitutions can be made provided that replacing the hydrogen with the substituent does not introduce unacceptable instability to the molecules of the present invention, and is otherwise chemically reasonable. One of ordinary skill in the art would understand that with respect to any chemical group described as optionally substituted, only sterically practical and/or synthetically feasible chemical groups are meant to be included. “Optionally substituted” as used herein refers to all subsequent modifiers in a term or series of chemical groups. For example, in the term “optionally substituted arylalkyl,” the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

“Reaction” as used herein refers to a process in which one or more substances or compounds or substrates is converted into one or more different substances, compounds, or processes.

### Enzymatic Synthesis of Natural and Modified Nucleoside Tetraphosphates (NQPs)

### 3′O-Kinase Mediated NQP Synthesis

New methods to synthesize natural and modified NQPs are necessary to enable production of oligonucleotides on the scale required for modern synthetic biology applications.

The present disclosure provides methods to synthesize natural and modified NTPs using one or more enzymes. In some embodiments, the present disclosure provides enzymes for the conversion of an NTP to an NTP with a phosphate group at the 3′ position of the sugar (known herein as a nucleoside tetraphosphate, pppNp, or NQP), as depicted in Scheme 1.

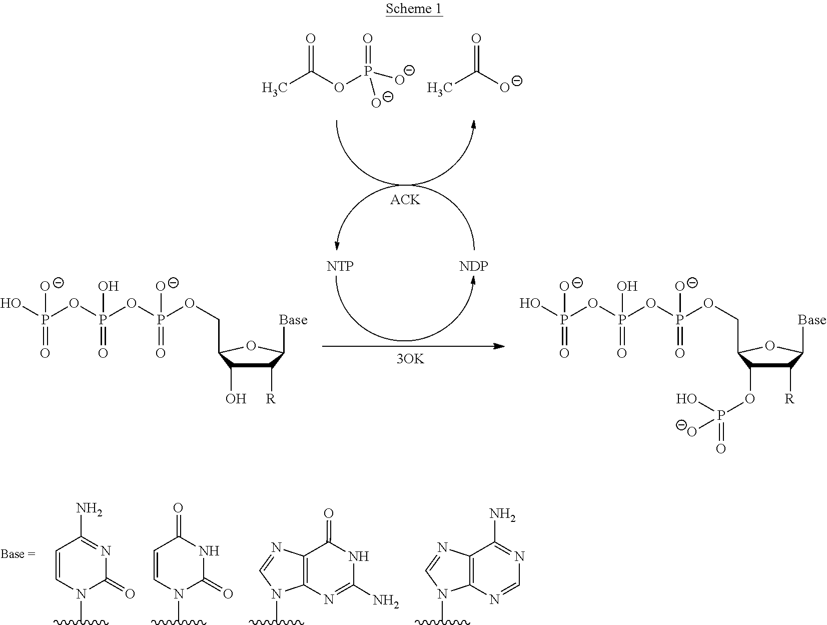

As depicted in Scheme 1, the NTP is converted by a 3′O-kinase to a nucleoside tetraphosphate (NQP or pppNp) with the fourth phosphate group at the 3′ position of the sugar. The group R at the 2′ position of the sugar (“2′-R group”) may be an atom or group selected from H, OH, OCH₃, OCH₂CH₂OCH₃ F, and CO₂R′ (where R′ is any alkyl or aryl), or another atom or chemical group. Additionally, the sugar may have other modifications at other positions. The nucleobase may be a uridine, thymine, cytosine, adenine, guanine or another nucleobase known to those skilled in the art. Although not depicted in Scheme 1, the NTP may also have modifications of the nucleobase or of the 5′ phosphate chain. Any of these modifications may be present in any combination in the 3′O-kinase substrate or may be added after or during conversion to the 3′O-kinase product.

In the embodiment depicted in Scheme 1, the 3′O-kinase uses an NTP as a co-substrate and phosphate donor, simultaneously producing an NDP. An acetate kinase enzyme (ACK) is used to recycle the NTP donor from NDP using acetyl-phosphate as a donor substrate that is converted to acetate. In some embodiments, the phosphate donor NTP is a different type of NTP than the substrate NTP (e.g. ATP donor versus GTP substrate or ATP donor versus 2′-F-ATP substrate). However, this is only one embodiment of the present invention, which is not intended to be so limited. The phosphate used by the 3′O-kinase may be sourced from any suitable molecule, with or without a recycling enzyme, as is known by those of skill in the art.

In certain embodiments where the 3′O-kinase enzyme is coupled with an ACK recycling enzyme, the ACK enzyme may be further coupled with a pyruvate oxidase enzyme (POX) to generate acetyl phosphate from pyruvate. In certain embodiments, the POX enzyme transiently generates acetyl phosphate from pyruvate, atmospheric oxygen, and potassium phosphate buffer. These embodiments have the advantage of generating an unstable, moisture sensitive, and expensive substrate (acetyl phosphate) from stable, readily available, and inexpensive reagents (pyruvate, atmospheric oxygen, and potassium phosphate buffer).

In some embodiments, the 3′O-kinase may catalyze one or more side reactions. In some embodiments, the side reaction produces a byproduct instead of, or in addition to an NQP. As depicted in Scheme 2, below, the byproduct may comprise a fourth phosphate on the 5′-OH phosphate chain (adenosine-5′-tetraphosphate or ppppN, denoted herein as p4A), or it may comprise a 3′ phosphate with an additional or fourth phosphate on the 5′-OH phosphate chain (3′O-phosphoadenosine-5′-tetraphosphate or ppppNp), or it may comprise an additional phosphate at the 2′ position (2′, 3′O-phosphoadenosine-5′-tetraphosphate) or 3′ position (3′O-diphospohoadenosdine-5′triphosphate) of the sugar.

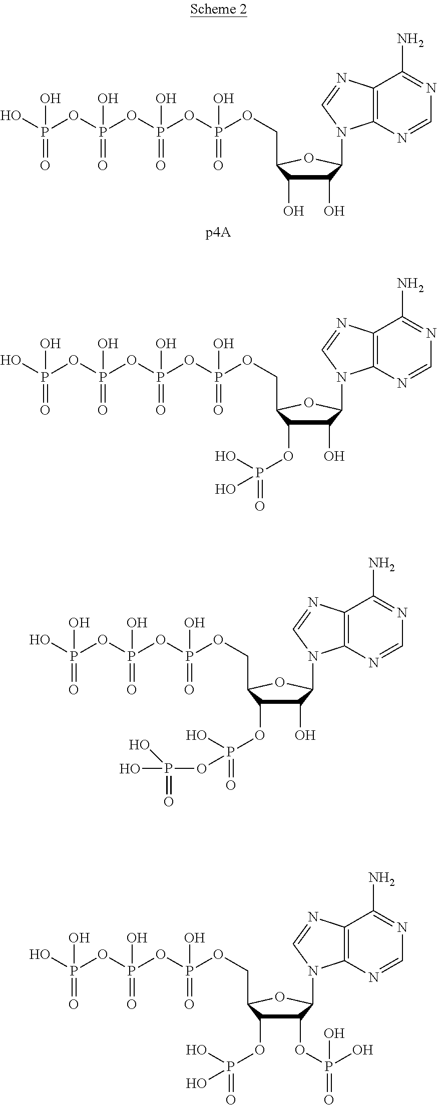

In some embodiments, the 3′O-kinase is more selective for the production of NQP as compared to the p4A or other byproduct species. In some embodiments, the 3′O-kinase is more selective for the production p4A or other byproduct species as compared to NQP. In some embodiments, the 3′O-kinase is 100% selective for NQP. In some embodiments, the 3′O-kinase is 100% selective for p4A.

Any suitable 3′O-kinase may be used in the present invention. Various suitable 3′O-kinases are known in the art. These include homologs of CysC enzymes (adenylyl-sulfate kinase) and CoaE (dephospho-CoA kinase). As used herein, the term 3′O-kinase refers to any of these enzymes and any enzyme capable of phosphorylation of the 3′ position of a natural or modified NTP, NDP, NMP, nucleoside, or nucleoside analog. CysC enzymes catalyze the conversion of adenosine 5′-phosphosulfate to 3′-phosphoadenylyl sulfate using ATP as a co-factor (C. Satishchandran et al., J. Biol. Chem., 1989, 264(25), 15012-15021)

Similarly, CoaE enzymes are known to catalyze the conversion of 3′-dephospho-CoA to CoA, using ATP as a co-factor (Satishchandran C. et al. Biochemistry 1992, 31, 47, 11684-11688). Various 3′O-kinase enzymes that may be used in the present invention are presented, below, in the Examples.

In some embodiments, the present disclosure provides novel 3′O-kinases that have improved activity in the conversion of an NTP to an NQP or pppNp (an NTP with a phosphate group at the 3′ position of the sugar). The 3′O-kinases of the present disclosure have increased activity on natural substrates, increased activity on non-natural substrates, increased selectivity, increased substrate promiscuity, decreased product inhibition and/or decreased byproduct formation, as compared to a wild-type or reference 3′O-kinase. The engineered polypeptides of the present disclosure are variants of SEQ ID NO: 10, a wild-type dephospho-CoA kinase (CoaE) from the species Geobacillus stearothermophilus. These engineered 3′O-kinases are capable of improved activity in the production of NQPs, using the methods described herein.

In some embodiments, the present invention provides an engineered 3′O-kinase polypeptide comprising an amino acid sequence having at least 60% sequence identity to an amino acid reference sequence of SEQ ID NO: 10 and further comprising one or more amino acid residue differences as compared to the reference amino acid sequence, wherein the engineered 3′O-kinase polypeptide has increased activity, increased activity on non-natural substrates, increased selectivity, increased substrate promiscuity, decreased product inhibition, and/or decreased byproduct formation, as compared to a wild-type or reference 3′O-kinase.

In particular, the engineered 3′O-kinase polypeptides of the present disclosure have been engineered for efficient synthesis of NQPs, in the processes depicted in Scheme 1, above, and Schemes 3 and 4, below. A variety of suitable reaction conditions are known to those skilled in the art, as detailed below and in the Examples.

In some embodiments, the present disclosure provides a method of producing an NTP with a phosphate group at the 3′ position of the sugar giving an NQP (pppNp), the method comprising (i) providing a 3′O-kinase enzyme, and (ii) contacting the 3′O-kinase enzyme with an NTP under suitable reaction conditions, such that an NTP with a phosphate group at the 3′ position of the NQP (pppNp) is produced.

In any of the above embodiments, the method may further comprise an engineered 3′O-kinase comprising one or more amino acid residue substitutions as compared to a wild type or reference polypeptide. In any of the above embodiments, the method may further comprise a nucleoside, NMP, NDP, NTP, and/or NQP with one or more modifications to the sugar, the 5′ phosphate chain, or nucleobase. In any of the above embodiments, the method may further comprise providing a source of phosphate, and, optionally, one or more phosphate recycling enzymes, including but not limited to pyruvate oxidase and/or acetate kinase.

### One Pot Synthesis: One Step and Two Step Methods

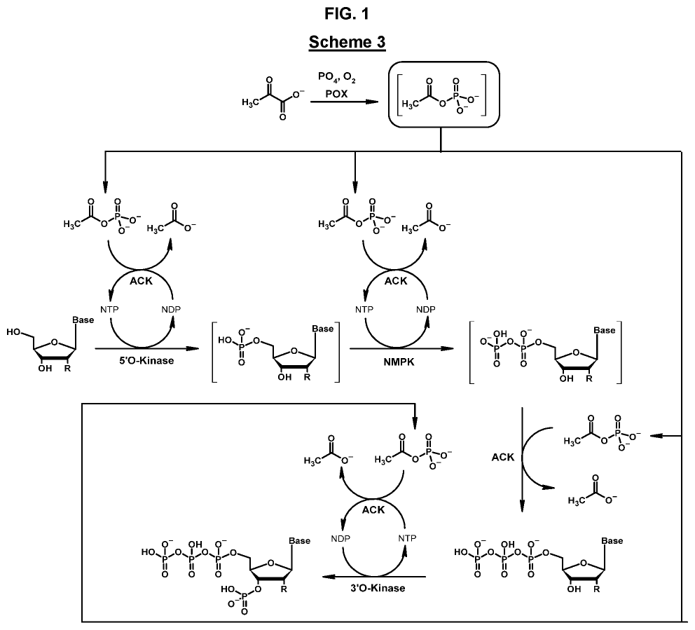

In some embodiments, the present disclosure provides a one-pot method for conversion of nucleosides to NQPs, as depicted in Scheme 3 (shown in FIG. 1). In some embodiments, the one-pot method for conversion of nucleosides to NQPs occurs in one step.

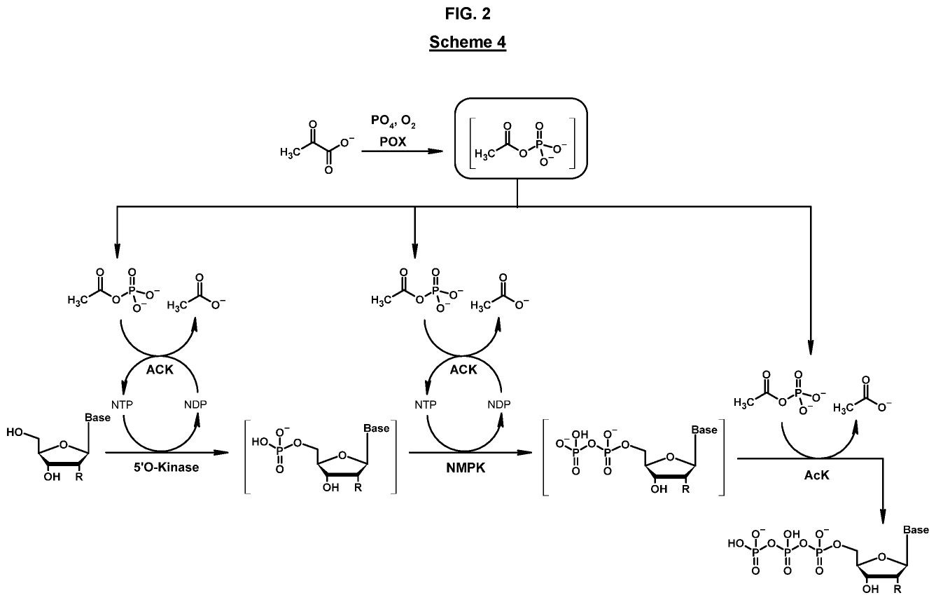

In some embodiments, the present disclosure provides a one-pot method for conversion of nucleosides to NQPs, as depicted in Scheme 3. In some embodiments, the one-pot method for conversion of nucleosides to NQPs occurs in more than one step. In some embodiments, the one-pot method for conversion of nucleosides to NQPs occurs in two steps. In some embodiments, the first step comprises conversion of a nucleoside to an NTP (as depicted in Scheme 4 shown in FIG. 2), and the second step comprises conversion of the NTP to an NQP (as depicted in Scheme 1, above). In some other embodiments, the first step comprises conversion of a nucleoside to a nucleoside with a phosphate at the 3′ position, and the second step comprises conversion of the nucleoside with a phosphate at the 3′ position to an NQP.

The first step of the two step one-pot method for conversion of nucleosides to NQPs comprises use of a 5′O-kinase, a nucleotide monosphate kinase (NMPK), and an acetate kinase (ACK) to sequentially add three phosphates (or modified phosphates or phosphate substitutes) to a nucleoside to generate an NTP.

As depicted in Scheme 4, the nucleoside is first converted by a 5′O-kinase to an NMP by addition of a phosphate group to the 5′-OH position of the sugar. After conversion of the nucleoside to an NMP by the 5′O-kinase, the NMP is converted to an NDP by an NMPK. Then, the NDP is converted to an NTP by an ACK.

With reference to Scheme 4, the 2′-R group may be H, OH, O—CH₃, F, OCH₂CH₂OCH₃, CO₂R′ (where R′ is any alkyl or aryl), or another atom or chemical group. Additionally, the sugar may have other modifications at other positions. The nucleobase may be a uridine, thymine, cytosine, adenine, guanine or another nucleobase known to those skilled in the art. Although not depicted in Scheme 4, the nucleoside may also have modifications of the nucleobase or of the 5′ phosphate chain. Any of these modifications may be present in any combination in any of the substrates or products depicted in Scheme 4 or may be added after conversion to the NTP product or may be added during or after the second step of the conversion depicted in Scheme 1.

In the embodiment depicted in Scheme 4, the 5′O-kinase and NMPK use an NTP as a co-substrate and phosphate donor, simultaneously producing an NDP. In addition to catalyzing the conversion of the NDP to NTP, the ACK is used to recycle the NTP donor from NDP using acetyl phosphate as a donor substrate that is converted to acetate. However, this is only one embodiment of the present invention, which is not intended to be so limited. The phosphate used by the 5′O-kinase and NMPK may be sourced from any suitable molecule, with or without a recycling enzyme, as is known by those of skill in the art.

In certain embodiments where the 5′O-kinase and NMPK enzymes are coupled with an ACK recycling enzyme, the ACK enzyme may be further coupled with a pyruvate oxidase enzyme (POX) to generate acetyl phosphate from pyruvate, as depicted in Scheme 4. In certain embodiments, the POX enzyme transiently generates acetyl phosphate from pyruvate, atmospheric oxygen, and potassium phosphate buffer. These embodiments have the advantage of generating an unstable, moisture sensitive, and expensive substrate (acetyl phosphate) from stable, readily available, and inexpensive reagents (pyruvate, atmospheric oxygen, and potassium phosphate buffer).

The second step of the two step one-pot method for conversion of nucleosides to NQPs comprises use of a 3′O-kinase to convert the natural or modified NTP generated in step one to the NQP product. The second step of the two step one-pot method conversion of nucleosides to NQPs, is described above and depicted in Scheme 1.

Thus, the two step one-pot method for conversion of nucleosides to NQPs comprises i) a first step comprising providing a 5′OK enzyme, an NMPK enzyme, and an ACK enzyme under suitable reaction conditions for conversion of a natural or modified nucleoside to a natural or modified NTP, optionally including an ACK recycling enzyme and/or POX enzyme and/or other suitable recycling enzymes; ii) a second step comprising providing a 3′OK enzyme under suitable reaction conditions for conversion of a natural or modified NTP to a natural or modified NQP, optionally including an ACK recycling enzyme and/or POX enzyme or other suitable recycling enzymes; and iii) optionally, comprising providing one or more additional enzymes or catalysts to generate one or more modifications to a nucleoside, NMP, NDP, NTP, or NQP as part of the one-pot method.

The one step one-pot method for conversion of nucleosides to NQPs comprises i) a step comprising providing a 5′OK enzyme, an NMPK enzyme, an ACK enzyme, and a 3′OK enzyme under suitable reaction conditions for conversion of a natural or modified nucleoside to a natural or modified NQP, optionally including an ACK enzyme and/or POX enzyme and/or other suitable recycling enzymes; and ii) optionally, comprising providing one or more additional enzymes or catalysts to generate one or more modifications to a nucleoside, NMP, NDP, NTP, or NQP as part of the one-pot method.

Any suitable 5′O-kinase may be used in the present invention. Various suitable 5′O-kinases are known in the art. These include homologs of adenosine kinase (AdoK) and polynucleotide 5′-hydroxyl-kinaseAs used herein, the term 5′O-kinase refers to any of these enzymes and any enzyme capable of phosphorylation of the 5′ position of a natural or modified nucleoside. Various 5′O-kinase enzymes that may be used in the present invention are presented, below, in the Examples.

Any suitable NMPK may be used in the present invention. Various suitable NMPKs are known in the art. These include homologs of adenylate kinase (AdK) and guanylate kinase. As used herein, the term NMPK refers to any of these enzymes and any enzyme capable of phosphorylation of the 5′beta phosphate position of a natural or modified NDP. Various NMPK enzymes that may be used in the present invention are presented, below, in the Examples.

Any suitable ACK may be used in the present invention. Various suitable ACK are known in the art. These include homologs of ACK. In some embodiments, more than one ACK is used. In some embodiments, one ACK is used for conversion of a natural or modified NDP to the desired NTP product, and a different ACK is used as a recycling enzyme for the conversion of NDPs to NTPs used as cofactors in the 5′OK and NMPK reactions. Various ACK enzymes that may be used in the present invention are presented, below, in the Examples.

Any suitable POX may be used in the present invention. Various suitable POX are known in the art. These include homologs of POX. As used herein, the term POX refers to any of these enzymes and any enzyme capable of decarboxylative phosphorylation of pyruvate to generate acetyl phosphate. Various POX enzymes that may be used in the present invention are presented, below, in the Examples.

In some embodiments, the present disclosure provides a method of producing an NTP with a phosphate group at the 3′ position of the sugar or NQP (pppNp), the method comprising (i) providing a 5′O-kinase enzyme, a nucleoside monophosphate kinase enzyme, an acetate kinase enzyme, and a 3′O-kinase enzyme, and (ii) contacting the 5′O-kinase enzyme, the nucleoside monophosphate kinase enzyme, the acetate kinase enzyme, and the 3′O-kinase enzyme with a nucleoside under suitable reaction conditions, such that an NTP with a phosphate group at the 3′ position of the sugar or NQP (pppNp) is produced.

In some embodiments, the present disclosure provides a method of producing an NTP with a phosphate group at the 3′ position of the sugar or NQP (pppNp), the method comprising (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside under suitable reaction conditions, such that an NTP is produced; iii) providing a 3′O-kinase enzyme; and (iv) contacting the 3′O-kinase enzyme with the NTP under suitable reaction conditions, such that an NTP with a phosphate group at the 3′ position of the sugar or NQP (pppNp) is produced.

In some embodiments, the present disclosure provides a method of producing an NTP with a phosphate group at the 3′ position of the sugar or NQP (pppNp), the method comprising i) providing a 3′O-kinase enzyme; (ii) contacting the 3′O-kinase enzyme with a nucleoside under suitable reaction conditions, such that a nucleoside with a phosphate at the 3′ position of the sugar is produced; (iii) providing a 5′O-kinase enzyme, a nucleoside monophosphate kinase enzyme, and an acetate kinase enzyme; and (iv) contacting the 5′O-kinase enzyme, the nucleoside monophosphate kinase enzyme, and the acetate kinase enzyme with a nucleoside with nucleoside with a phosphate at the 3′ position of the sugar under suitable reaction conditions, such that an NTP with a phosphate group at the 3′ position of the sugar or NQP (pppNp) is produced.

In any of the above embodiments, the method may further comprise an engineered 3′O-kinase comprising one or more amino acid residue substitutions as compared to a wild type or reference polypeptide. In any of the above embodiments, the method may further comprise a nucleoside, NMP, NDP, NTP, and/or NQP with one or more modifications. In any of the above embodiments, the method may further comprise providing a source of phosphate, and, optionally, one or more phosphate recycling enzymes, including but not limited to pyruvate oxidase and/or acetate kinase. In any of the above embodiments, the method may further comprise providing more than one 3′O-kinase in step (i).

In some embodiments, the present disclosure provides enzymes for the conversion of a nucleoside to an NMP via addition of a phosphate group to the 5′ position of the sugar. In some embodiments, the present invention provides enzymes for the conversion of an NMP to an NDP. In some embodiments, the present disclosure provides enzymes for the conversion of an NDP to an NTP. In some embodiments, the present disclosure provides enzymes for the conversion of an NTP to an NQP. In some embodiments, the present disclosure provides a one-pot method for conversion of nucleosides to NQPs. In some embodiments, the present disclosure provides a one-pot method, two step method for conversion of nucleosides to NQPs.

In any of the above embodiments, the nucleosides, NMPs, NDPs, NTPs, NQPs, p4A and other byproduct species may be natural or may comprise one or more modifications. In any of the above embodiments, the nucleosides, NMPs, NDPs, NTPs, NQPs, p4A and other byproduct species may comprise ribonucleosides, deoxyribonucleosides, dideoxynucleosides, or modified nucleosides. In any of the above embodiments, the nucleosides, NMPs, NDPs, NTPs, NQPs, p4A and other byproduct species may comprise one or more modifications to the sugar. In any of the above embodiments, the nucleosides, NMPs, NDPs, NTPs, NQPs, p4A and other byproduct species may comprise one or more modifications to the nucleobase. In any of the above embodiments, the NMPs, NDPs, NTPs, NQPs, p4A and other byproduct species may comprise an α-thiophosphate or dithiophosphate or other modification to the 5′ phosphate chain.

### Engineered 3′O-Kinase Polypeptides

The present invention provides engineered 3′O-kinase polypeptides useful in the synthesis of NQPs, as well as compositions and methods of utilizing these engineered polypeptides.

The present invention provides 3′O-kinase polypeptides, polynucleotides encoding the polypeptides, methods of preparing the polypeptides, and methods for using the polypeptides. Where the description relates to polypeptides, it is to be understood that it can describe the polynucleotides encoding the polypeptides.

Suitable reaction conditions under which the above-described improved properties of the engineered polypeptides carry out the desired reaction can be determined with respect to concentrations or amounts of polypeptide, substrate, co-substrate, buffer, solvent, pH, conditions including temperature and reaction time, and/or conditions with the polypeptide immobilized on a solid support, as further described below and in the Examples.

In some embodiments, exemplary engineered 3′O-kinases comprise an amino acid sequence that has one or more residue differences as compared to SEQ ID NO: 10 at the residue positions indicated in Tables 13.1, 13.2, 13.3, 13.4, and 13.5.

The structure and function information for the exemplary engineered polypeptides of the present invention are based on the conversion of a natural or modified NTP to a natural or modified NQP, the results of which are shown below in Tables 13.1, 13.2, 13.3, 13.4, and 13.5, as further described in the Examples. The odd numbered sequence identifiers (i.e., SEQ ID NOs) in these Tables refer to the nucleotide sequence encoding the amino acid sequence provided by the even numbered SEQ ID NOs in these Tables. Exemplary sequences are provided in the electronic sequence listing file accompanying this invention, which is hereby incorporated by reference herein. The amino acid residue differences are based on comparison to the reference sequence of SEQ ID NO: 10.

Various 3′O-kinases, have been identified in many species. These include homologs of CysC enzymes (adenylyl-sulfate kinase) and CoaE (dephospho-CoA kinase). CysC enzymes catalyze the conversion of adenosine 5′-phosphosulfate to 3′-phosphoadenylyl sulfate using ATP as a co-factor. Similarly, CoaE enzymes are known to catalyze the conversion of 3′-dephospho-CoA to CoA, using ATP as a co-factor. Other 3′O-kinases are also known in the art and may be used to practice the invention. As used herein, the term 3′O-kinase refers to any of these enzymes and any enzyme capable of phosphorylation of the 3′ position of a natural or modified NTP, NDP, NMP, or nucleoside.

The wild-type 3′O-kinase (CoaE) from Geobacillus stearothermophilus (SEQ ID NO: 10) was selected for evolution. The 3′O-kinase polypeptides of the present disclosure are engineered variants of SEQ ID NO: 10.

The polypeptides of the present disclosure have residue differences that result in improved properties necessary to develop an efficient 3′O-kinase enzyme, capable of biocatalytic synthesis of NQPs. Various residue differences, at both conserved and non-conserved positions, have been discovered to be related to improvements in various enzymes properties, including increased activity, increased activity on non-natural substrates, increased selectivity, increased substrate promiscuity, decreased product inhibition, and/or decreased byproduct formation, as compared to a wild-type or reference 3′O-kinase. Increased activity of the 3′O-kinase polypeptide may be evidenced by increased % conversion of substrate to product. The activity of each engineered 3′O-kinase relative to the reference polypeptide of SEQ ID NO: 10 was determined as conversion of the substrates described in the Examples herein. In some embodiments, a shake flask purified enzyme (SFP) is used to assess the properties of the engineered 3′O-kinases, the results of which are provided in the Examples.

In some embodiments, the specific enzyme properties are associated with the residues differences as compared to SEQ ID NO: 10 at the residue positions indicated herein. In some embodiments, residue differences affecting polypeptide expression can be used to increase expression of the engineered 3′O-kinases.

In light of the guidance provided herein, it is further contemplated that any of the exemplary engineered polypeptides comprising the even-numbered sequences of SEQ ID NOs: 56-366, or 372-2122 find use as the starting amino acid sequence for synthesizing other 3′O-kinase polypeptides, for example by subsequent rounds of evolution that incorporate new combinations of various amino acid differences from other polypeptides in Tables 13.1, 13.2, 13.3, 13.4, and 13.5, and other residue positions described herein. Further improvements may be generated by including amino acid differences at residue positions that had been maintained as unchanged throughout earlier rounds of evolution.

In some embodiments, the engineered 3′O-kinase comprises a polypeptide sequence having at least 60%, 70%, 80%, 85%, 90%, 91%, 92%, 93%, 94%, 95%, 96%, 97%, 98%, 99%, or more sequence identity to a reference sequence selected from SEQ ID NO: 10, 142, 372, 450, 496, 1042, 1180, 1412 1464, 1800, and 2078, or a functional fragment thereof, and one or more amino acid residue differences relative to the reference sequence.

In some embodiments, a reference sequence, as well as any specified amino acid sequence herein, can be described without the amino acid residues of a His-tag when present. For example, an polypeptide sequence of an engineered 3′O-kinase comprises residues 1-201 of an engineered 3′O-kinase referenced by it SEQ ID NO., where the sequence of the SEQ ID NO. includes a His-tag. It is also to be understood that the range of residues can be adapted to account for any amino acid deletions within the sequence of the 3′O-kinase polypeptide sequence.

As such, in some embodiments, the present disclosure provides an engineered 3′O-kinase comprising a polypeptide sequence having at least 60%, 70%, 80%, 85%, 90%, 91%, 92%, 93%, 94%, 95%, 96%, 97%, 98%, 99%, or more sequence identity to a reference sequence corresponding to residues 1-201 of SEQ ID NO: 10, 142, 372, 450, 496, 1042, 1180, 1412 1464, 1800, and 2078, and one or more amino acid residue differences relative to the reference sequence.

In some embodiments, the engineered 3′O-kinase comprises a polypeptide sequence having at least an amino acid residue difference at amino acid position 2, 3, 4, 5, 6, 7, 10, 11, 13, 15, 17, 19, 20, 22, 23, 25, 26, 27, 28, 29, 32, 35, 36, 38, 39, 40, 41, 44, 46, 47, 48, 49, 50, 51, 52, 53, 55, 56, 57, 58, 59, 60, 61, 62, 63, 64, 67, 68, 69, 71, 72, 74, 76, 77, 78, 79, 81, 82, 83, 84, 85, 86, 88, 89, 91, 92, 93, 94, 95, 96, 97, 98, 100, 101, 103, 104, 105, 109, 110, 111, 115, 116, 117, 121, 122, 123, 124, 125, 126, 127, 129, 130, 131, 134, 135, 136, 138, 139, 141, 142, 144, 146, 148, 149, 150, 152, 153, 156, 157, 158, 160, 161, 163, 165, 166, 167, 169, 170, 171, 173, 175, 176, 177, 178, 179, 181, 182, 184, 190, 191, 192, 194, 195, 196, 197, 198, 199, 200, 201, 202, 203, 204, 210, or 211, or combinations thereof, relative to the reference sequence of SEQ ID NO: 10, 142, 372, 450, 496, 1042, 1180, 1412 1464, 1800, and 2078.

In some embodiments, the engineered 3′O-kinase comprises a polypeptide sequence having an amino acid residue difference or amino acid residue 2L/V, 3C/D/K/L/P/V, 4G/V, 5T/Y, 6C/F/W, 7I/M/S, 10S, 11K, 13A/D/E/V, 15S, 17Q/R/S, 19C/G, 20C/T, 22A/E/S, 23A, 25A/M/V, 26T, 27F/Y, 28D/N/T/V, 29A/C, 32G/K/W, 35M/S, 36G/R/S/T, 38E/S/V, 39C/L, 40M, 41A/K/M/V, 44A/C/D/G/P, 46T, 47G/K/S, 48H, 49C/D/G/H/M/V, 50E/M/S/V, 51L/T, 52I, 53E/F/K/N/R, 55Y, 56E/M, 57A/C/W, 58C/L/T, 59S, 60C/L/V, 61E/Y, 62G, 63E/I/S/T, 64R/V, 67F, 68G/Q/R, 69R, 71A/I/Q/R/T/V/Y, 72D/H/K/L/N/Q/R/S/T, 74A/C/F/G/I/K/M/R, 76S, 76A, 77N, 78A/M, 79G/N, 81L, 82E/L/M/T, 83M, 84P, 85A/G/L/P/R/V, 86S, 88L/M, 89G/L/S, 91E, 92A/T, 93C/E/F/I/L/M/Q/T/V/Y, 94A/C, 95H/I/L/S/Y, 96I/T, 97A/N, 98I/L, 100I/R/S/W, 1011, 103C/F/I/L/M/V/Y, 104A/C/F/G/N/R/T/Y, 105A/E/G/I/K/L/M/S, 109L/R/S, 110A, 1111, 115V/W, 116S, 117G, 121L, 122A/I, 123A/I/V, 124A/H/Q/S/V/W/Y, 125C/M/P/Q/R/S/Y, 126A/C/G/L/M/P/V, 127C/M/Y, 129S, 130Q, 131I, 134H/L, 135D/F, 136A/F, 138-/S, 139-/A, 141I/P, 142D/K/Y, 144V, 146D/H/S/T, 148G/R/V, 149F/K/L/P/T, 150L/M/W, 152D/I, 153D/S/V, 156M/P, 157E/K/M, 158H, 160D/E/K/L/S, 161V, 163S/V, 1651/Q/S, 166E/I/L, 167D/I/T, 1691, 170A/C/G/I/P/Y, 171G/L/M/S/T/V, 173A/H/P/V/Y, 175W, 176G/L, 177S, 178H/M/R/S, 179M, 181N/V, 182C/E/P, 184D, 190V, 191C/D/T, 192L, 194E/G, 195P/S, 196Y, 197K/V, 1981/L, 199C/T/V, 200C/D/E/N, 201A/C/H/N/S, 202D/I/P/T, 203A/C/L/R, 204A/C/L/M/R/T, 210E/P/Q/S/T/V, or 211K, or combinations thereof, relative to the reference sequence of SEQ ID NO: 10, 142, 372, 450, 496, 1042, 1180, 1412 1464, 1800, and 2078.

In some embodiments, the engineered 3′O-kinase comprises a polypeptide sequence having at least 60%, 70%, 80%, 85%, 90%, 91%, 92%, 93%, 94%, 95%, 96%, 97%, 98%, 99%, or more sequence identity to a reference sequence selected from SEQ ID NO: 10, 142, 372, 450, 496, 1042, 1180, 1412 1464, 1800, and 2078, or a functional fragment thereof, and one or more amino acid residue differences relative to the reference sequence of SEQ ID NO: 10.

In some embodiments, the engineered 3′O-kinase comprises a polypeptide sequence having at least 60%, 70%, 80%, 85%, 90%, 91%, 92%, 93%, 94%, 95%, 96%, 97%, 98%, 99%, or more sequence identity to a reference sequence selected from an even-numbered sequence selected from SEQ ID NO: 56-366 and 372-2122, and one or more amino acid residue differences relative to the reference sequence of SEQ ID NO: 10.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 13, 38, 39, 72, 74, 89, 93, 124, and 165. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 13A, 13V, 38E, 39L, 72R, 74K, 89L, 93Y, 124V, 124W, and 165S. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from S13A, S13V, A38E, V39L, A72R, V74K, H89L, R93Y, T124V, T124W, and M165S. In the above embodiments, the engineered 3′O-kinase polypeptide may additionally comprise improved conversion of an NTP to an NQP, as compared to a reference sequence of SEQ ID NO: 10.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 17, 41, 60, 123, 138, 138/139, 144, 148, 150, 163, 165, 177, 178, and 179. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 17R, 41M, 60L, 123A, 123I, 138-/139-, 138S, 144V, 148G, 148R, 150M, 163S, 165S, 177S, 178H, 178R, and 179M. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected T17R, R41M, R60L, L123A, L123I, D138-/V139-, D138S, L144V, N148G, N148R, F150M, W163S, M165S, D177S, N178H, N178R, and N179M. In the above embodiments, the engineered 3′O-kinase polypeptide may additionally comprise improved conversion of an NTP to p4A, as compared to a reference sequence of SEQ ID NO: 10.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 13, 32, 35, 36, 39, 40, 74, 76, 89, 92, 150, and 156. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 13V, 32G, 35S, 36T, 39L, 40M, 74K, 74M, 76A, 89G, 89L, 89S, 92A, 150W, and 156M. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from S13V, A32G, A35S, A36T, V39L, V40M, V74K, V74M, N76A, H89G, H89L, H89S, V92A, F150W, and L156M. In the above embodiments, the engineered 3′O-kinase polypeptide may additionally comprise improved selectivity for the NQP product, as compared to a reference sequence of SEQ ID NO: 10.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 17, 41, 116, 123, 138/139, 144, 148, 150, 177, 178, and 179. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 17Q, 17R, 41M, 116S, 123A, 123I, 123V, 138-/139-, 144V, 148G, 148R, 150M, 177S, 178H, 178R, 178S, and 179M. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from T17Q, T17R, R41M, P116S, L123A, L123I, L123V, D138-/V139-, L144V, N148G, N148R, F150M, D177S, N178H, N178R, N178S, and N179M. In the above embodiments, the engineered 3′O-kinase polypeptide may additionally comprise improved selectivity for the p4A or other byproduct species product, as compared to a reference sequence of SEQ ID NO: 10.

As will be appreciated by the skilled artisan, in some embodiments, one or a combination of residue differences above that is selected can be kept constant (i.e., maintained) in the engineered 3′O-kinase as a core feature, and additional residue differences at other residue positions incorporated into the sequence to generate additional engineered 3′O-kinase polypeptides with improved properties. Accordingly, it is to be understood for any engineered 3′O-kinase containing one or a subset of the residue differences above, the present invention contemplates other engineered 3′O-kinase that comprise the one or a subset of the residue differences, and additionally one or more residue differences at the other residue positions disclosed herein.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 13/36/38/39/40/74/89, 13/36/38/39/40/89, 13/36/38/39/72/76/89/93/124, 13/36/38/40, 13/36/38/40/72/74, 13/36/38/40/72/74/89/93, 13/36/38/40/72/74/93/156, 13/36/38/40/72/74/124, 13/36/38/40/72/76/89, 13/36/38/40/74/76, 13/36/38/40/76/89/93, 13/36/38/40/89, 13/36/39/40/72/76/89/124, 13/36/40/72/74/76/93, 13/36/40/72/156, 13/36/40/93/124, 13/38/39/40/72/76/93/165, 13/38/39/40/89/124/156, 13/38/40/89, 13/38/72/156, 13/40, 13/40/72/76/89, 13/72, 13/72/74/76/89/93, 13/72/74/76/89/93/124, 13/72/74/89/93, 13/72/74/89/93/124, 13/72/76, 13/72/76/89/93, 13/72/76/89/124, 13/72/76/124/156, 13/72/89, 13/72/89/93/124/156, 13/72/89/124, 13/72/89/124/165, 13/72/93/124, 13/72/124, 13/74/89/93, 13/74/89/93/124, 13/74/89/156, 13/76, 13/76/89/93, 13/76/89/93/124, 13/76/89/93/156/165, 13/76/89/124/156, 13/76/89/156/165, 13/76/93, 13/76/93/124, 13/76/124, 13/89, 13/89/124, 13/89/165, 13/124, 13/156, 36/38/39/40/72/74/76/124, 36/38/39/40/72/74/89, 36/38/39/40/74/76/89/93/124, 36/38/40/72/74/89/93/124/156, 36/39/40/72/76/89/93, 36/39/40/76/93/156, 36/40/72/74/89/93, 38/39, 38/39/40/72/74/76/89, 38/39/76, 38/40/72, 38/40/76/89/124, 38/40/89/124, 38/40/93, 38/40/156, 38/72/76/89, 38/72/89/93/124, 38/76/156, 39/40/72/76, 72/74/76/89/124, 72/74/76/124, 72/74/89, 72/74/89/93, 72/74/89/93/124, 72/74/93, 72/74/93/124, 72/76, 72/76/89/93, 72/76/124, 72/89/165, 72/93/124, 74/76/89, 74/76/89/93/124, 74/76/89/124, 74/89, 74/89/93, 74/89/93/156, 74/89/124, 76/89/93/124, 76/89/93/165, 76/89/124/165, 76/89/165, 76/93, 89/93, 89/93/124, 89/124, and 124/156. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from 13A/36T/38E/40M, 13A/36T/38E/40M/72R/76A/89G, 13A/36T/38E/40M/76A/89S/93Y, 13A/36T/40M/72R/74K/76A/93Y, 13A/36T/40M/93Y/124V, 13A/38E/40M/89G, 13A/38E/72R/156M, 13A/40M/72R/76A/89L, 13A/72R/76A, 13A/72R/89G, 13A/72R/89L/124W, 13A/72R/89S/93Y/124V/156M, 13A/74K/89G/156M, 13A/76A, 13A/76A/124W, 13A/89L/124V, 13A/89L/165S, 13A/89S/165S, 13A/124V, 13V/36T/38E/39L/40M/74K/89G, 13V/36T/38E/39L/40M/89L, 13V/36T/38E/39L/72R/76A/89L/93Y/124W, 13V/36T/38E/40M/72R/74K, 13V/36T/38E/40M/72R/74K/89L/93Y, 13V/36T/38E/40M/72R/74K/93Y/156M, 13V/36T/38E/40M/72R/74K/124V, 13V/36T/38E/40M/74K/76A, 13V/36T/38E/40M/89G, 13V/36T/39L/40M/72R/76A/89L/124V, 13V/36T/40M/72R/156M, 13V/38E/39L/40M/72R/76A/93Y/165S, 13V/38E/39L/40M/89L/124W/156M, 13V/38E/40M/89G, 13V/40M, 13V/72R, 13V/72R/74K/76A/89L/93Y, 13V/72R/74K/76A/89L/93Y/124V, 13V/72R/74K/89L/93Y, 13V/72R/74K/89L/93Y/124W, 13V/72R/76A, 13V/72R/76A/89G/93Y, 13V/72R/76A/89L/124V, 13V/72R/76A/124V/156M, 13V/72R/89G, 13V/72R/89L/124V/165S, 13V/72R/89L/124W, 13V/72R/93Y/124W, 13V/72R/124W, 13V/72S/74K/89L/93Y, 13V/74K/89L/93Y, 13V/74K/89L/93Y/124V, 13V/74K/89S/93Y, 13V/76A, 13V/76A/89G/93Y/124W, 13V/76A/89G/156M/165S, 13V/76A/89L/93Y/156M/165S, 13V/76A/89L/124W/156M, 13V/76A/89S/93Y, 13V/76A/93Y, 13V/76A/93Y/124V, 13V/89G, 13V/89L, 13V/89S, 13V/124V, 13V/124W, 13V/156M, 36T/38E/39L/40M/72R/74K/76A/124W, 36T/38E/39L/40M/72R/74K/89L, 36T/38E/39L/40M/74K/76A/89L/93Y/124V, 36T/38E/40M/72R/74K/89S/93Y/124W/156M, 36T/39L/40M/72R/76A/89L/93Y, 36T/39L/40M/76A/93Y/156M, 36T/40M/72R/74K/89G/93Y, 38E/39L, 38E/39L/40M/72R/74K/76A/89S, 38E/39L/76A, 38E/40M/72R, 38E/40M/76A/89L/124W, 38E/40M/89S/124W, 38E/40M/93Y, 38E/40M/156M, 38E/72R/89L/93Y/124V, 38E/76A/156M, 38V/72R/76A/89G, 39L/40M/72R/76A, 72R/74K/76A/89G/124V, 72R/74K/76A/124V, 72R/74K/89G, 72R/74K/89G/93Y, 72R/74K/89L/93Y/124V, 72R/74K/93Y, 72R/74K/93Y/124V, 72R/76A, 72R/76A/89G/93Y, 72R/76A/124V, 72R/76A/124W, 72R/89L/165S, 72R/93Y/124V, 74K/76A/89G/124V, 74K/76A/89L, 74K/76A/89L/93Y/124V, 74K/89L, 74K/89L/93Y/156M, 74K/89L/124W, 74K/89S/93Y, 76A/89G/93Y/124V, 76A/89G/93Y/165S, 76A/89G/165S, 76A/89L/124V/165S, 76A/93Y, 89L/93Y, 89L/93Y/124W, 89L/124W, and 124W/156M. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 10 and one or more residue differences as compared to SEQ ID NO: 10, selected from S13A/A36T/A38E/V40M, S13A/A36T/A38E/V40M/A72R/N76A/H89G, S13A/A36T/A38E/V40M/N76A/H89S/R93Y, S13A/A36T/V40M/A72R/V74K/N76A/R93Y, S13A/A36T/V40M/R93Y/T124V, S13A/A38E/V40M/H89G, S13A/A38E/A72R/L156M, S13A/V40M/A72R/N76A/H89L, S13A/A72R/N76A, S13A/A72R/H89G, S13A/A72R/H89L/T124W, S13A/A72R/H89S/R93Y/T124V/L156M, S13A/V74K/H89G/L156M, S13A/N76A, S13A/N76A/T124W, S13A/H89L/T124V, S13A/H89L/M165S, S13A/H89S/M165S, S13A/T124V, S13V/A36T/A38E/V39L/V40M/V74K/H89G, S13V/A36T/A38E/V39L/V40M/H89L, S13V/A36T/A38E/V39L/A72R/N76A/H89L/R93Y/T124W, S13V/A36T/A38E/V40M/A72R/V74K, S13V/A36T/A38E/V40M/A72R/V74K/H89L/R93Y, S13V/A36T/A38E/V40M/A72R/V74K/R93Y/L156M, S13V/A36T/A38E/V40M/A72R/V74K/T124V, S13V/A36T/A38E/V40M/V74K/N76A, S13V/A36T/A38E/V40M/H89G, S13V/A36T/V39L/V40M/A72R/N76A/H89L/T124V, S13V/A36T/V40M/A72R/L156M, S13V/A38E/V39L/V40M/A72R/N76A/R93Y/M165S, S13V/A38E/V39L/V40M/H89L/T124W/L156M, S13V/A38E/V40M/H89G, S13V/V40M, S13V/A72R, S13V/A72R/V74K/N76A/H89L/R93Y, S13V/A72R/V74K/N76A/H89L/R93Y/T124V, S13V/A72R/V74K/H89L/R93Y, S13V/A72R/V74K/H89L/R93Y/T124W, S13V/A72R/N76A, S13V/A72R/N76A/H89G/R93Y, S13V/A72R/N76A/H89L/T124V, S13V/A72R/N76A/T124V/L156M, S13V/A72R/H89G, S13V/A72R/H89L/T124V/M165S, S13V/A72R/H89L/T124W, S13V/A72R/R93Y/T124W, S13V/A72R/T124W, S13V/A72S/V74K/H89L/R93Y, S13V/V74K/H89L/R93Y, S13V/V74K/H89L/R93Y/T124V, S13V/V74K/H89S/R93Y, S13V/N76A, S13V/N76A/H89G/R93Y/T124W, S13V/N76A/H89G/L156M/M165S, S13V/N76A/H89L/R93Y/L156M/M165S, S13V/N76A/H89L/T124W/L156M, S13V/N76A/H89S/R93Y, S13V/N76A/R93Y, S13V/N76A/R93Y/T124V, S13V/H89G, S13V/H89L, S13V/H89S, S13V/T124V, S13V/T124W, S13V/L156M, A36T/A38E/V39L/V40M/A72R/V74K/N76A/T124W, A36T/A38E/V39L/V40M/A72R/V74K/H89L, A36T/A38E/V39L/V40M/V74K/N76A/H89L/R93Y/T124V, A36T/A38E/V40M/A72R/V74K/H89S/R93Y/T124W/L156M, A36T/V39L/V40M/A72R/N76A/H89L/R93Y, A36T/V39L/V40M/N76A/R93Y/L156M, A36T/V40M/A72R/V74K/H89G/R93Y, A38E/V39L, A38E/V39L/V40M/A72R/V74K/N76A/H89S, A38E/V39L/N76A, A38E/V40M/A72R, A38E/V40M/N76A/H89L/T124W, A38E/V40M/H89S/T124W, A38E/V40M/R93Y, A38E/V40M/L156M, A38E/A72R/H89L/R93Y/T124V, A38E/N76A/L156M, A38V/A72R/N76A/H89G, V39L/V40M/A72R/N76A, A72R/V74K/N76A/H89G/T124V, A72R/V74K/N76A/T124V, A72R/V74K/H89G, A72R/V74K/H89G/R93Y, A72R/V74K/H89L/R93Y/T124V, A72R/V74K/R93Y, A72R/V74K/R93Y/T124V, A72R/N76A, A72R/N76A/H89G/R93Y, A72R/N76A/T124V, A72R/N76A/T124W, A72R/H89L/M165S, A72R/R93Y/T124V, V74K/N76A/H89G/T124V, V74K/N76A/H89L, V74K/N76A/H89L/R93Y/T124V, V74K/H89L, V74K/H89L/R93Y/L156M, V74K/H89L/T124W, V74K/H89S/R93Y, N76A/H89G/R93Y/T124V, N76A/H89G/R93Y/M165S, N76A/H89G/M165S, N76A/H89L/T124V/M165S, N76A/R93Y, H89L/R93Y, H89L/R93Y/T124W, H89L/T124W, and T124W/L156M. In the above embodiments, the engineered 3′O-kinase polypeptide may additionally comprise improved conversion of an NTP to an NQP and/or improved selectivity for the NQP product, as compared to a reference sequence of SEQ ID NO: 10.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 142 and one or more residue differences as compared to SEQ ID NO: 142 at a position or set of positions selected from 13/76/93, 13/76/93/198, 13/76/198, 68, 68/103/181/182, 76, 82, 82/198, 83, 86, 88, 91, 93, 93/198, 103, 111, 169, 181, 182, 191, 200, 210, and 211. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 142 and one or more residue differences as compared to SEQ ID NO: 142 selected from S13E/A76S/Y93Q, S13E/A76S/Y93Q/A198L, S13E/A76S/A198L, A68Q, A68R, A68G/H103V/T181N/I182E, A76S, K82E, K82T/A198I, V83M, A86S, V88L, A91E, Y93C, Y93E, Y93F, Y93I, Y93L, Y93M, Y93Q, Y93V, Y93L/A198L, H103L, H103Y, V111, V169I, T181N, 1182E, A191D, G200C, G200D, G200E, H210E, H210P, H210Q, H210S, H210T, H210V, and H211K.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 372 and one or more residue differences as compared to SEQ ID NO: 372 at a position or set of positions selected from 13/40/68/74/93/157, 13/40/68/157, and 40/68/81. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 372 and one or more residue differences as compared to SEQ ID NO: 372 selected from E13A/V40M/A68Q/V74R/Q93L/A157K, E13A/V40M/A68R/A157K, and V40M/A68R/R81L.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 at a position or set of positions selected from 41/46/190/191, 41/83/86/181/190/191, 41/86/181/191, 41/181/191, 41/46/86/95/111, 41/46/86/95/191, 41/86/181/190/191, 41/86/181/191, 41/95/111/181/190/191, 41/190, 46/83/190/191, 46/86/181/190/191, 46/190/191, 48, 48/81, 48/81/103, 48/103/175/200, 48/103/200, 48/135, 48/135/175, 48/135/200, 48/200, 72, 72/82/88/124/166, 72/82/166, 72/82/91/124/166/182, 72/124/166, 72/166, 72/166/182, 72/182, 81, 81/103, 81/135, 81/135/200, 81/175/200, 81/200, 82, 82/88/91/124/166/182, 82/88/91/182, 82/88/124/166, 82/124/166, 82/124/166/182, 82/166/182, 86, 88/166, 91, 91/124/166/182/201, 91/166, 103, 103/135, 103/135/200, 103/175, 103/175/200, 103/200, 124, 124/166, 124/166/182, 135, 135/175/200, 135/200, 166, 166/182, 175, 175/200, 182, and 200.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 selected from R41K/A46T/A86S/K951/V1111, R41K/A46T/A86S/K95L/A191D, R41V/A46T/L190V/A191D, R41V/V83M/A86S/T181N/L190V/A191D, R41K/A86S/T181N/L190V/A191D, R41K/A86S/T181N/A191D, R41K/K95I/V111I/T181N/L190V/A191D, R41V/T181N/A191D, R41K/L190V, A46T/V83M/L190V/A191D, A46T/A86S/T181N/L190V/A191D, A46T/L190V/A191D, R48H, R48H/R81L, R48H/R81L/H103L, R48H/R81L/H103L/V175W/G200C, R48H/H1031/G200N, R48H/H103L/G200C, R48H/H103L/G200E, R48H/H103V/V175W/G200C, R48H/V135D, R48H/V135D/V175W, R48H/V135D/G200E, R48H/G200N, A72H, A72H/K82E/A91E, A72H/K82L/A166I, A72H/K82T/V88L/T124Q/A166E, A72H/T124Q/A1661, A72H/A166E, A72H/A166I/I182E, A72H/I182E, A72Q/K82E/A91E/T124S/A166E/I182E, R81L, R81L/H103I, R81L/H103L, R81L/H103M, R81L/V135D, R81L/V135D/G200D, R81L/V175W/G200E, R81L/V175W/G200N, R81L/G200C, R81L/G200D, K82E/V88L/A91E/I182E, K82E/A166E/I182E, K82L/V88M/A91E/T124Q/A166E/I182E, K82L/T124Q/A166L, K82L/T124S/A166I/I182E, K82L/A166E/I182E, K82M/T124Q/A166E, K82T, K82T/V88L/T124Q/A166E, A86S, V88L/A166E, A91E, A91E/T124Q/A166E/I182E/K201N, A91E/A166E, H103F/G200N, H103I, H103I/V135D, H1031/V175W, H103L/V175W/G200D, H103L, H103L/V135D/G200C, H103M, T124Q/A166E/1182E, T124Q/A1661, T124S, V135D, V135D/V175W/G200N, V135D/G200E, A166L, A166E/1182E, V175W, V175W/G200D, 1182E, G200C, G200D, and G200N.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 at a position or set of positions selected from 3, 61, 105, 125, 126, 142, and 171. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 selected from F3V, A61E, R105K, H125Q, W126C, W126L, W126V, R142K, and R171M.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 at a position or set of positions selected from 2, 3, 7, 19, 22, 25, 28, 29, 32, 35, 36, 44, 49, 50, 51, 57, 58, 63, 77, 78, 85, 97, 98, 100, 104, 105, 121, 122, 125, 126, 136, 153, 167, 170, 191, 201, 202, 203, and 204. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 selected from A2L, F3C, F3D, F3K, F3L, L7I, L7M, S19C, S19G, M22A, L25A, P28T, P28V, V29C, A32G, A32W, A35M, A36R, E44C, Q49G, Q49H, Q49M, I50M, V51L, G57A, I58C, I58L, G63E, D77N, E78A, E78M, N85A, N85G, N85L, N85R, L97A, L97N, A98I, K100R, K100W, I104T, R105A, S121L, G122A, H125C, H125M, H125P, H125R, W126A, W126C, W126G, D136A, E153D, E153V, E167D, K170A, A191C, K201A, K201C, G202D, G203C, S204A, S204L, and S204T.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 at a position or set of positions selected from 2, 3, 4, 5, 6, 7, 13, 15, 17, 22, 25, 26, 28, 29, 32, 35, 36, 44, 47, 49, 50, 51, 52, 53, 55, 56, 57, 58, 59, 60, 61, 63, 77, 79, 84, 85, 92, 94, 97, 98, 100, 101, 104, 105, 109, 122, 125, 126, 127, 129, 136, 139, 142, 149, 152, 153, 167, 170, 171, 173, 191, 194, 195, 196, 197, 199, 201, 202, 203, and 204. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 450 and one or more residue differences as compared to SEQ ID NO: 450 selected from A2L, A2V, F3C, F3L, F3P, F3V, T4G, T4V, IST, I5Y, G6C, G6F, G6W, L7I, L7S, A13D, K15S, T17S, M22A, M22E, M22S, L25M, L25V, G26T, P28D, P28N, P28T, V29C, A32G, A32K, A32W, A35S, A36G, A36S, E44A, E44D, E44G, E44P, Y47G, Y47K, Y47S, Q49C, Q49V, I50E, I50M, I50S, I50V, V51L, V51T, A52I, A53E, A53F, A53K, A53R, G55Y, P56E, P56M, G57C, G57W, I58T, L59S, R60C, R60L, R60V, A61E, A61Y, G63I, G63S, G63T, D77N, Q79G, L84P, N85P, N85V, V92T, K94A, K94C, L97N, A98I, K100I, K100S, E101I, 1104A, 1104C, 1104G, 1104R, 1104T, 1104Y, R105E, R105G, R105I, R105K, R105L, R105M, R105S, K109L, K109R, K109S, G122I, H125C, H125P, H125Q, H125R, H125Y, W126A, W126C, W126G, W126L, W126M, W126P, W126V, V127C, V127Y, K129S, D136F, V139A, R142D, R142Y, G149F, G149K, G149L, G149P, G149T, E152D, E1521, E153S, E1671, E167T, K170C, K170G, K1701, K170P, K170Y, R171G, R171L, R171S, R171T, R171V, D173H, D173P, D173V, D173Y, A191T, H194E, H194G, Q195P, Q195S, W196Y, D197K, D197V, L199C, L199T, L199V, K201S, G202D, G202I, G202P, G202T, G203A, G203L, G203R, S204A, S204C, S204R, and S204T.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 496 and one or more residue differences as compared to SEQ ID NO: 496 at a position or set of positions selected from 3/49/61/81/83/124/125/166/171, 3/49/61/81/124/125/200, 3/49/61/83, 3/49/61/83/125/166, 3/49/61/83/200, 3/49/61/124/125/171/200, 3/49/81/83/124/125/171, 3/49/81/83/124/125/200, 3/49/81/124/125/166/171, 3/49/105/124/125/200, 3/49/124/125/166/171/200, 3/49/166/171, 3/61/81/105/124/125/166/171, 3/61/81/125/166/171/200, 3/81/105/124/166, 3/81/124/125/200, 3/83/166/171, 3/166/171, 49/61/81/83, 49/61/83/105/124/125/171, 49/61/83/124/125/171, 49/61/124/125/166/171, 49/61/125/171, 49/83/105/111/124, 49/83/105/124/125/166, 49/111/124/166/171/200, 49/124/125/166, 50/60/72/86/103, 50/60/82/83/103/126/142/175/191, 50/91/126/135, 60/182, 61/81/83/166/171/200, 61/81/125/166/171/200, 61/83/124/125/200, 61/124/125/166/171/200, 61/125, 61/166, 61/200, 72/82/83/142/181/191/200, 72/86/91/97/135, 72/142/182, 82/83, 82/83/103, 83/91/94/95/126/135/191, 83/105/124/125/166/200, 83/105/166, 83/125/171, 86/94/111/126/142, 86/126/135/142, 94/126, 105, 111/126/135/175/182, 124/125, 126, 142, 182, and 200. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 496 and one or more residue differences as compared to SEQ ID NO: 496 selected from F3V/Q49D/A61E/R81L/V83M/T124S/H125S/A166I/R171M, F3V/Q49D/A61E/R81L/T124Q/H125Q/G200D, F3V/Q49D/A61E/R81L/T124Q/H125S/G200D, F3V/Q49D/A61E/V83M, F3V/Q49D/A61E/V83M/H125S/A166E, F3V/Q49D/A61E/V83M/G200D, F3V/Q49D/A61E/T124Q/H125Q/R171M/G200D, F3V/Q49D/R81L/V83M/T124Q/H125S/R171M, F3V/Q49D/R81L/V83M/T124S/H125S/G200D, F3V/Q49D/R81L/T124Q/H125Q/A166E/R171M, F3V/Q49D/R105K/T124Q/H125S/G200D, F3V/Q49D/T124Q/H125Q/A166I/R171M/G200D, F3V/Q49D/A166E/R171M, F3V/A61E/R81L/R105K/T124Q/H125S/A1661/R171M, F3V/A61E/R81L/H125S/A166I/R171M/G200D, F3V/R81L/R105K/T124Q/A166I, F3V/R81L/T124S/H125S/G200D, F3V/V83M/A166I/R171M, F3V/A166E/R171M, Q49D/A61E/R81L/V83M, Q49D/A61E/V83M/R105K/T124S/H125S/R171M, Q49D/A61E/V83M/T124S/H125S/R171M, Q49D/A61E/T124Q/H125S/A166I/R171M, Q49D/A61E/H125S/R171M, Q49D/V83M/R105K/V111I/T124Q, Q49D/V83M/R105K/T124Q/H125Q/A166I, Q49D/V111I/T124Q/A166E/R171M/G200D, Q49D/T124Q/H125Q/A166I, I50V/R60L/A72Q/S86A/H103L, I50V/R60L/K82E/V83M/H103M/W126L/R142K/V175W/D191A, I50V/A91E/W126V/V135D, R60L/I182E, A61E/R81L/V83M/A166I/R171M/G200D, A61E/R81L/H125S/A166E/R171M/G200D, A61E/V83M/T124Q/H125S/G200D, A61E/T124S/H125S/A166E/R171M/G200D, A61E/H125Q, A61E/A166E, A61E/G200D, A72Q/K82E/V83M/R142K/N181T/D191A/G200N, A72Q/S86A/A91E/L97N/V135D, A72Q/R142K/I182E, K82E/V83M, K82E/V83M/H103F, V83M/A91E/K94A/K951/W126L/V135D/D191A, V83M/R105K/T124S/H125S/A1661/G200D, V83M/R105K/A166E, V83M/H125S/R171M, S86A/K94A/V111I/W126L/R142K, S86A/W126L/V135D/R142K, K94A/W126L, R105K, V111I/W126V/V135D/V175W/1182E, T124Q/H125S, W126V, R142K, 1182E, and G200D.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 496 and one or more residue differences as compared to SEQ ID NO: 496 at a position or set of positions selected from 3/49/61/83/200, 3/49/105/124/125/200, 72/82/83/142/181/191/200, 126, 142, and 191/200. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 496 and one or more residue differences as compared to SEQ ID NO: 496 selected from F3V/Q49D/A61E/V83M/G200D, F3V/Q49D/R105K/T124Q/H125S/G200D, A72Q/K82E/V83M/R142K/N181T/D191A/G200N, W126V, R142K, and D191A/G200D.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 at a position or set of positions selected from 7, 7/61/85/97/126, 7/85/97/126, 13, 19, 19/53/105, 19/53/201, 19/100/105/201, 32/85/126/204, 35/50, 50/78/142, 53/58/100/105/109, 53/58/109/201, 53/100/105, 61, 79, 79/126/204, 85, 85/97, 97, 100, 105/201, 126, 171/201, and 204. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 selected from L71, L71/N85G/L97A/W126M, L7S/A61E/N85R/L97A/W126M, A13D, S19G, S19G/A53N/K105G, S19G/A53N/K201H, S19G/K100R/K105L/K201H, A32W/N85R/W126G/S204C, A35M/I50E, I50M/E78A/R142D, A53E/158T/K100R/K105G/K109S, A53E/K100W/K105G, A53N/158T/K109S/K201S, A61E, Q79G, Q79G/W126M/S204C, N85A, N85R/L97N, L97A, K100R, K105G/K201H, W126A, W126M, R171M/K201S, and S204M.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 at a position or set of positions selected from 7/28/32/97, 7/61, 28/32/61, 28/32/71/79/97/126/204, 28/32/85, 28/32/97/126, 28/36/61, 32/36/61/85/97/126/204, 32/36/126, 32/85/126/204, 36/61/126/204, 53/100/105, 85, 85/97, 85/126, and 100/105. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 selected from L71/P28T/A32W/L97N, L71/A61E, P28T/A32W/A61E, P28T/A32W/G71V/Q79G/L97A/W126M/S204C, P28T/A32W/N85R, P28T/A32W/L97A/W126M, P28T/A36R/A61E, A32W/A36G/A61E/N85G/L97N/W126M/S204C, A32W/A36G/W126M, A32W/N85R/W126G/S204C, A36R/A61E/W126M/S204C, A53E/K100W/K105G, N85A, N85R/L97N, N85R/W126M, K100W/K105A, and K100W/K105G.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 at a position or set of positions selected from 23, 27, 62, 67, 69, 71, 71/131, 72, 74, 93, 95, 103, 115, 117, 124, 134, 141, 146, 150, 156, 157, 158, 160, 161, 165, 166, 176, and 182. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 selected from R23A, L27F, L27Y, D62G, R67F, K69R, G71A, G71I, G71T, G71Y/L1311, A72D, A72K, A72N, A72T, R74F, R74G, R74I, L93T, K95S, H103F, H103M, H103V, I115V, 1115W, L117G, Q124H, Y134H, Y134L, L141I, L141P, A146D, A146S, A146T, F150L, L156P, K157E, K157M, R158H, R160D, R160L, S161V, S1651, S165Q, A166E, A166L, I176G, I176L, I182C, and I182E.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 at a position or set of positions selected from 20, 27, 41, 68, 71, 72, 74, 89, 96, 103, 110, 115, 124, 150, 157, 160, 165, 181, 182, and 192. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 selected from A20T, L27F, K41A, Q68G, G71A, G71Q, G71R, G71T, G71V, A72D, R74A, R74C, R74F, R741, R74V, G89L, M961, H103L, H103M, T110A, I115V, Q124Y, F150L, K157M, R160E, S165I, S165Q, N181T, 1182E, and 1192L.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 at a position or set of positions selected from 10, 11, 20, 38, 39, 64, 72, 74, 95, 96, 103, 115, 117, 124, 130, 135, 146, 148, 160, 161, 163, 175, 176, 178, 181, and 182. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1042 and one or more residue differences as compared to SEQ ID NO: 1042 selected from G10S, 111K, A20C, A20T, A38S, V39C, E64R, E64V, A72L, R74G, K95H, K95Y, M96T, H103C, H103F, I115W, L117G, Q124A, V130Q, V135F, A146H, N148V, R160K, R160S, S161V, W163V, V175W, I176L, N178M, N181V, and 1182P.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1180 and one or more residue differences as compared to SEQ ID NO: 1180 at a position or set of positions selected from 3/25/29/60/170, 3/25/29/126, 3/25/126, 3/44/126/170, 25/44/58, 25/58/60, 44/58/60/61/126/170, 58/61/126, 127, 167/171/173, and 170. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1180 and one or more residue differences as compared to SEQ ID NO: 1180 selected from V3D/L25A/V29A/R60C/K170A, V3D/L25A/V29A/W126V, V3D/L25A/W126M, V3D/E44C/W126M/K170A, L25A/I58L/R60L, L25M/E44C/158C, E44D/158L/R60L/A61E/W126C/K170A, I58L/A61E/W126M, V127M, E167D/R171L/D173A, and K170A.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1180 and one or more residue differences as compared to SEQ ID NO: 1180 at a position or set of positions selected from 17/63, 17/63/104, 17/63/104/125, 22/55/98/127, 22/55/98/167/171/173/197, 25/29/60/126, 25/36/126, 28, 44/60/61/126, 59/104/125, 63/125, 79/125/129, 98/167/171, and 127. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1180 and one or more residue differences as compared to SEQ ID NO: 1180 selected from T17S/G63E, T17S/G63S/I104F/S125R, T17S/G63S/I104N, M22A/G55Y/A98I/V127M, M22A/G55Y/A98L/E167D/R171V/D173A/D197V, L25A/V29A/R60C/W126L, L25A/A36R/W126V, P28V, E44C/R60L/A61E/W126M, L59S/I104F/S125R, G63S/S125Y, Q79N/S125R/K129S, A98I/E167D/R171M, and V127M.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 at a position or set of positions selected from 27, 27/68, 27/68/71/184, 27/71, 27/71/184, 27/95, 41/72/160/161, 68/71/113, 68/71/113/157/176, 68/71/157/184, 71, and 71/184. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 selected from L27F, L27F/Q68G, L27F/Q68G/G71A/E184D, L27F/G71Q, L27F/G71T/E184D, L27F/K95H, L27F/K95Y, K41A/A72D/R160S/S161V, Q68G/G71Q/L113M/K157R/I176L, Q68G/G71R/L113M, Q68G/G71R/K157M/E184D, G71A, G71A/E184D, and G71T/E184D.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 at a position or set of positions selected from 49, 52, 61, 83, and 125. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 selected from D49M, D49Q, A52S, A61G, V83R, S125A, and S125E.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 at a position or set of positions selected from 2, 3, 4, 13, 28, 30, 32, 35, 36, 40, 43, 45, 48, 49, 51, 52, 53, 54, 56, 57, 60, 61, 63, 76, 77, 78, 80, 81, 82, 83, 85, 86, 90, 92, 94, 97, 100, 101, 104, 109, 121, 127, 129, 133, 139, 152, 153, 154, 167, 173, 186, 191, 194, 197, and 198. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 selected from A2L, A2Q, A2R, A2V, A2W, V3A, V3G, V3S, T4P, T4Q, A13M, A13V, P28E, I30W, A32W, A35L, A35R, A35V, A35Y, A36E, A36G, A36P, A36S, M40R, G43E, E45L, R48E, R48P, R48V, D49G, D49M, D49N, D49V, V51A, A52D, A52L, A52Q, A52T, E53G, F54M, P56G, P56K, P56L, G57K, G57L, G57V, R60S, R60V, A61H, G63F, G63Q, S76D, S76G, S76T, D77E, D77W, E78G, E78V, Q80T, Q80V, R81W, K82D, K82H, V83E, V83L, V83Q, N85A, N85M, N85R, S86Q, P90R, V92A, V92T, K94A, K94G, K94N, K94Q, K94T, L97G, L97T, L97W, W100N, W100R, E101L, I104A, I104L, K109D, K109S, S121E, M127A, M127P, K129P, V133N, V139L, V139Q, V139R, V139T, E152L, E153V, E154Q, E154T, E167A, D173A, D173I, D173P, R186A, R186P, R186V, D191G, H194M, D197A, L198G, and L198P.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 at a position or set of positions selected from 2, 19, 35, 45, 48, 49, 52, 53, 61, 76, 78, 80, 83, 85, 98, 106, 109, 121, 125, 170, 171, 194, and 195. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 selected from A2V, A2W, S19T, A35C, E45Q, R48G, R48K, D49G, D49N, D49Q, D49R, D49S, A52K, E53K, A61E, A61G, S76D, E78P, Q80G, V83R, V83T, N85A, N85M, N85R, N85W, A98G, S106R, K109A, S121T, S125E, K170C, K170I, K170T, R171A, R171M, R171Q, H194A, Q195A, and Q195K.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 at a position or set of positions selected from 28, 40, 48, 49, 51, 57, 60, 80, 82, 83, 92, 94, 98, 100, 104, 109, 127, 171, 186, 193, 194, 195, and 198. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1412 and one or more residue differences as compared to SEQ ID NO: 1412 selected from P28E, M40R, R48A, R48C, R48E, R48G, R48K, R48T, R48V, D49L, V51I, G57A, G57K, R60S, R60V, Q80T, K82H, V83L, V92A, V92T, K94A, K94G, K94R, A98G, W100N, I104V, K109D, K109M, K109S, M127T, R171L, R186S, R186V, L193V, H194A, Q195A, and L198S.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 at a position or set of positions selected from 3/20/74/103, 10/23/27/38/49/113, 10/27/38/49, 10/83, 23/27/49/83/125/141, 27/49/74, 27/60/83/125, 27/83/113, 39, 41/64/72/103/160, 41/64/103/117/150/160/161, 49/60, 49/64/96/113/175, 49/68/134, 60/61, 60/175, 61/110/146/151, 64, 64/72/115/150, 64/103/150/181, 64/150/181, 64/161, 68/72/83/175, 72/103/124/160/161, 72/103/125/150/160/181, 72/124/150/160/181, 74/165, 103/182, 117/150, 150/160/181, 150/181, 151, 160/181, 182, and 192. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 selected from V3T/A20C/R74F/H103F, G10S/R23A/L27F/A38S/D49Q/L113A, G10S/L27F/A38S/D49M, G10S/V83R, R23A/L27F/D49Q/V83R/S125E/L141I, L27F/D49Q/R74H, L27F/R60A/V83R/S125E, L27F/V83R/L113A, V39C, K41A/E64R/A72D/H103M/R160K, K41A/E64R/H103M/L117G/F150L/R160K/S161V, D49M/R60A, D49M/G68S/Y134M, D49Q/E64A/M96T/L113A/V175E, R60A/V175E, R60L/A61P, A61P/T110A/A146H/T151G, E64R, E64R/A72D/I115V/F150L, E64R/H103M/F150L/N181T, E64R/F150L/N181T, E64R/S161V, G68S/A72L/V83R/V175E, A72D/H103M/Q124A/R160K/S161V, A72D/H103M/S125A/F150L/R160E/N181S, A72D/Q124Y/F150L/R160S/N181V, R74F/S165I, H103F/I182E, H103L/I182P, L117G/F150L, F150L/R160E/N181V, F150L/N181S, T151G, R160K/N181T, 1182E, and 1192L.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 at a position or set of positions selected from 20/103/192, 52/61, 61/110/165, 64/72/115/150, 72, 72/103/125/150/160/181, and 192. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 selected from A20C/H103L/I192L, A52S/A61P, A61P/T110A/S165Q, E64R/A72D/I115V/F150L, A72D, A72D/H103M/S125A/F150L/R160E/N181S, and 1192L.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 at a position or set of positions selected from 16, 20, 35, 68, 75, 85, 88, 89, 93, 122, 127, 134, 139, 146, 148, 150, 151, 161, 165, 182, and 182/205. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 selected from S16T, A20H, A20R, A35L, G68E, F75L, N85L, N85T, V88L, G89L, L93V, G122E, M127V, Y134W, V139T, A146S, N148G, F150L, T151P, S161L, S165Q, 1182R, and 1182R/G205D.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 at a position or set of positions selected from 7, 18, 20, 22, 35, 67, 68, 71, 75, 81, 85, 88, 89, 121, 136, 137, 139, 141, 142, 146, 148, 150, 151, 153, 160, 161, 176, 182, 182/205, and 185. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 selected from L7V, V18L, A20G, A20H, M22L, A35L, A35V, R67K, G68E, G68S, G68Y, R71A, F75L, F75N, R81N, N85L, V88L, G89L, S121G, S121T, D136S, D137S, V139T, L141V, R142G, A146D, A146N, N148G, F150C, F150H, F150L, T151A, E153A, R160H, R160S, R160T, R160V, S161L, 1176L, 1182P, 1182R, 1182R/G205D, and T185A.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 at a position or set of positions selected from 18, 20, 23, 29, 30, 35, 36, 37, 38, 40, 71, 85, 89, 93, 95, 113, 127, 142, 146, 161, 165, and 185. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 selected from V18A, A20I, A20K, A20Q, A20S, R23A, V29I, 130G, 130V, A35D, A35L, A35M, A35R, A35V, A36S, R37V, A38I, M40L, M40R, R71A, N85E, G89M, G89T, L93V, K95V, L113W, M127A, M127V, R142A, R142S, A146Q, S161L, S165Q, and T185A.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 at a position or set of positions selected from 8, 11, 15, 88, 113, 133, 143, 155, and 161. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1464 and one or more residue differences as compared to SEQ ID NO: 1464 selected from T8R, I11P, K15Y, V88W, L113R, V133R, R143V, A155P, and S161V.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1800 and one or more residue differences as compared to SEQ ID NO: 1800 at a position or set of positions selected from 27, 27/49/51, 27/49/171, 40, 40/92/104, 48, 48/53/60/76/80/193, 48/56/60/76/167/170/193, 49, 53/56/60/76, 56/60, 56/60/76/78/80, 56/60/85/193, 56/76/80/170, 56/76/80/193, 56/85/104, 56/167/193, 60, 60/61, 60/193, 76/80, 98, 101, 101/109/198, 125, 165, 171/186, and 186. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1800 and one or more residue differences as compared to SEQ ID NO: 1800 selected from L27F, L27F/D49G/V51I, L27F/D49L/R171A, M40R, M40R/V92A/I104V, R48A, R48E/E53K/R60V/S76D/Q80T/L193V, R48E/P56L/R60V/S76D/E167A/K170T/L193V, D49N, D49Q, E53K/P56L/R60V/S76G, P56L/R60V, P56L/R60V/S76D/E78V/Q80T, P56L/R60V/N85M/L193V, P56L/S76D/Q80T/K170T, P56L/S76G/Q80T/L193V, P56L/N85M/I104V, P56L/E167A/L193V, R60A/A61E, R60V, R60V/L193V, S76G/Q80T, A98G, E101L, E101L/K109S/L198S, S125E, S165Q, R171L/R186P, and R186V.

In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2078 and one or more residue differences as compared to SEQ ID NO: 2078 at a position or set of positions selected from 48, 52, 100, 165, and 193. In some embodiments, the engineered 3′O-kinase polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2078 and one or more residue differences as compared to SEQ ID NO: 2078 selected from R48E, A52D, W100N, S165Q, and L193V.

As noted above, the engineered 3′O-kinase polypeptides are also capable of converting substrates (e.g., a natural or modified NTP) to products (e.g., an NQP). In some embodiments, the engineered 3′O-kinase polypeptide is capable of converting the substrate compounds to the product compound with at least 1.1, 1.2 fold, 1.5 fold, 2 fold, 3 fold, 4 fold, 5 fold, 10 fold, or more activity relative to the activity of the reference polypeptide of SEQ ID NO: 10.

In some embodiments, the engineered 3′O-kinase capable of converting the substrate compounds to the product compounds with at least 1.5 fold the activity relative to SEQ ID NO: 10, comprises an amino acid sequence selected from: the even-numbered sequences in SEQ ID NOs: 56-366, or 372-2122.

In some embodiments, the engineered 3′O-kinase has an amino acid sequence comprising one or more residue differences as compared to SEQ ID NO: 10, that increases soluble expression or isolated protein yield of the engineered 3′O-kinase in a bacterial host cell, particularly in E. coli, as compared to a wild-type or engineered reference 3′O-kinase, and comprises an amino acid sequence selected from the even-numbered sequences in SEQ ID NOs: 56-366, or 372-2122.

In some embodiments, the engineered 3′O-kinase has an amino acid sequence comprising one or more residue differences as compared to SEQ ID NO: 10 that increases selectivity for either the NQP product or the p4A product of the engineered 3′O-kinase, as compared to a wild-type or engineered reference 3′O-kinase, and comprises an amino acid sequence selected from the even-numbered sequences in SEQ ID NOs: 56-366, or 372-2122.

In some embodiments, the engineered 3′O-kinase has an amino acid sequence comprising one or more residue differences as compared to SEQ ID NO: 10 that increases activity of the engineered 3′O-kinase on one or more 2′ modified NTP substrates, as compared to a wild-type or engineered reference 3′O-kinase, and comprises an amino acid sequence selected from the even-numbered sequences in SEQ ID NOs: 56-366, or 372-2122.

In some embodiments, the engineered 3′O-kinase with improved properties has an amino acid sequence comprising a sequence selected from the even-numbered sequences in the range of SEQ ID NOs: 56-366, or 372-2122.

In some embodiments, the engineered 3′O-kinase, comprises an amino acid sequence having at least 80%, 85%, 86%, 87%, 88%, 89%, 90%, 91%, 92%, 93%, 94%, 95%, 96%, 97%, 98%, or 99% identity to one of the even-numbered sequences in the range of SEQ ID NOs: 56-366, or 372-2122, as provided in the Examples.

In addition to the residue positions specified above, any of the engineered 3′O-kinase polypeptides disclosed herein can further comprise other residue differences relative to SEQ ID NO:10, at other residue positions (i.e., residue positions other than those included herein). Residue differences at these other residue positions can provide for additional variations in the amino acid sequence without adversely affecting the ability of the polypeptide to carry out the conversion of substrate to product. Accordingly, in some embodiments, in addition to the amino acid residue differences present in any one of the engineered 3′O-kinase polypeptides selected from the even-numbered sequences in the range of SEQ ID NOs: 56-366, or 372-2122, the sequence can further comprise 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-100, or 1-150 residue differences at other amino acid residue positions as compared to the SEQ ID NO: 10. In some embodiments, the number of amino acid residue differences as compared to the reference sequence can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, 50, 100, or 150 residue positions. In some embodiments, the number of amino acid residue differences as compared to the reference sequence can be 1, 2, 3, 4, 5, 6, 7, 8, 9, 10, 11, 12, 13, 14, 15, 16, 18, 20, 21, 22, 23, 24, or 25 residue positions. The residue differences at these other positions can be conservative changes or non-conservative changes. In some embodiments, the residue differences can comprise conservative substitutions and non-conservative substitutions as compared to the 3′O-kinase polypeptide of SEQ ID NO: 10.

In some embodiments, the present invention also provides engineered polypeptides that comprise a fragment of any of the engineered 3′O-kinase polypeptides described herein that retains the functional activity and/or improved property of that engineered 3′O-kinase. Accordingly, in some embodiments, the present invention provides a polypeptide fragment capable of converting substrate to product under suitable reaction conditions, wherein the fragment comprises at least about 90%, 95%, 96%, 97%, 98%, or 99% of a full-length or truncated amino acid sequence of an engineered 3′O-kinase of the present invention, such as an exemplary 3′O-kinase polypeptide selected from the even-numbered sequences in the range of SEQ ID NOs: 56-366, or 372-2122. In some embodiments, the engineered 3′O-kinase can have an amino acid sequence comprising a deletion in any one of the 3′O-kinase polypeptide sequences described herein, such as the exemplary engineered polypeptides of the even-numbered sequences in the range of SEQ ID NOs: 56-366, or 372-2122.

Thus, for each and every embodiment of the engineered 3′O-kinase polypeptides of the invention, the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 20% of the total number of amino acids, or up to 30% of the total number of amino acids of the 3′O-kinase polypeptides, where the associated functional activity and/or improved properties of the engineered 3′O-kinase described herein are maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1,2,3,4,5, 6,7, 8,9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In some embodiments, the engineered 3′O-kinase polypeptide described herein can have an amino acid sequence comprising an insertion as compared to any one of the engineered 3′O-kinase polypeptides described herein, such as the exemplary engineered polypeptides of the even-numbered sequences in the range of SEQ ID NOs: 56-366, or 372-2122. Thus, for each and every embodiment of the 3′O-kinase polypeptides of the invention, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, 20 or more amino acids, 30 or more amino acids, 40 or more amino acids, or 50 or more amino acids, where the associated functional activity and/or improved properties of the engineered 3′O-kinase described herein is maintained. The insertions can be to amino or carboxy terminus, or internal portions of the 3′O-kinase polypeptide.

In some embodiments, the engineered 3′O-kinase described herein can have an amino acid sequence comprising a sequence selected from the even-numbered sequences in the range of SEQ ID NOs: 56-366, or 372-2122, and optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, 1-50, 1-75, 1-100, or 1-150 amino acid residue deletions, insertions and/or substitutions. In some embodiments, the amino acid sequence has optionally around 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, 50, 55, 60, 65, 70, 80, 90, 100, 110, 120, 130, 140, or 150 amino acid residue deletions, insertions and/or substitutions. In some embodiments, the substitutions can be conservative or non-conservative substitutions.

In the above embodiments, the suitable reaction conditions for the engineered polypeptides are provided as described in the Examples herein.

In some embodiments, the polypeptides of the present invention are fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the engineered polypeptides described herein can be used with or without fusions to other polypeptides.

It is to be understood that the polypeptides described herein are not restricted to the genetically encoded amino acids. In addition to the genetically encoded amino acids, the polypeptides described herein may be comprised, either in whole or in part, of naturally occurring and/or synthetic non-encoded amino acids. Certain commonly encountered non-encoded amino acids of which the polypeptides described herein may be comprised include, but are not limited to: the D-stereoisomers of the genetically-encoded amino acids; 2,3-diaminopropionic acid (Dpr); α-aminoisobutyric acid (Aib); s-aminohexanoic acid (Aha); 6-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aGly); propargylglycine (pgGly); homoalanine (hAla); norvaline (nVal); homoleucine (hLeu), homovaline (hVal); homoisoleucine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art (See e.g., the various amino acids provided in Fasman, CRC Practical Handbook of Biochemistry and Molecular Biology, CRC Press, Boca Raton, FL, pp. 3-70 [1989], and the references cited therein, all of which are incorporated by reference). These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(δ-benzylester), Gln(xanthyl), Asn(N-δ-xanthyl), His(bom), His(benzyl), His(tos), Lys(fmoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

In some embodiments, the engineered polypeptides can be in various forms, for example, such as an isolated preparation, as a substantially purified enzyme, whole cells transformed with gene(s) encoding the enzyme, and/or as cell extracts and/or lysates of such cells. The enzymes can be lyophilized, spray-dried, precipitated or be in the form of a crude paste, as further discussed below.

In some embodiments, the engineered polypeptides can be in the form of a biocatalytic composition. In some embodiments, the biocatalytic composition comprises (a) a means for conversion of a natural or modified NTP substrate to an NQP product by contact with a 3′O-kinase and (b) a suitable cofactor. The suitable cofactor may be another NTP or another suitable phosphate donor.

In some embodiments, the polypeptides described herein are provided in the form of kits. The enzymes in the kits may be present individually or as a plurality of enzymes. The kits can further include reagents for carrying out the enzymatic reactions, substrates for assessing the activity of enzymes, as well as reagents for detecting the products. The kits can also include reagent dispensers and instructions for use of the kits.

In some embodiments, the kits of the present invention include arrays comprising a plurality of different 3′O-kinase polypeptides at different addressable position, wherein the different polypeptides are different variants of a reference sequence each having at least one different improved enzyme property. In some embodiments, a plurality of polypeptides immobilized on solid supports are configured on an array at various locations, addressable for robotic delivery of reagents, or by detection methods and/or instruments. The array can be used to test a variety of substrate compounds for conversion by the polypeptides. Such arrays comprising a plurality of engineered polypeptides and methods of their use are known in the art (See e.g., WO2009/008908A2).

### Polynucleotides Encoding Engineered 3′O-Kinases, Expression Vectors and Host Cells

In another aspect, the present invention provides polynucleotides encoding the engineered 3′O-kinase polypeptides described herein. The polynucleotides may be operatively linked to one or more heterologous regulatory sequences that control gene expression to create a recombinant polynucleotide capable of expressing the polypeptide. Expression constructs containing a heterologous polynucleotide encoding the engineered 3′O-kinase are introduced into appropriate host cells to express the corresponding 3′O-kinase polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the improved 3′O-kinase enzymes. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the amino acid sequences presented in Tables 13.1, 13.2, 13.3, 13.4, and 13.5, and disclosed in the sequence listing incorporated by reference herein as the even-numbered sequences in the range of SEQ ID NOs: 56-366, or 372-2122.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. In some embodiments, all codons need not be replaced to optimize the codon usage of the 3′O-kinase since the natural sequence will comprise preferred codons and because use of preferred codons may not be required for all amino acid residues. Consequently, codon optimized polynucleotides encoding the 3′O-kinase enzymes may contain preferred codons at about 40%, 50%, 60%, 70%, 80%, or greater than 90% of codon positions of the full length coding region.

In some embodiments, the polynucleotide comprises a codon optimized nucleotide sequence encoding the 3′O-kinase polypeptide amino acid sequence, as represented by SEQ ID NO: 10. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences encoding the even-numbered sequences in the range of SEQ ID NOs: 56-366, or 372-2122. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences in the odd-numbered sequences in the range of SEQ ID NOs: 56-366, or 372-2122. In some embodiments, the codon optimized sequences of the odd-numbered sequences in the range of SEQ ID NOs: 55-365, or 371-2121, or 371-2121, enhance expression of the encoded 3′O-kinase, providing preparations of enzyme capable of converting substrate to product.

In some embodiments, the polynucleotides are capable of hybridizing under highly stringent conditions to a reference sequence selected from the odd-numbered sequences in SEQ ID NOs: 55-365, or 371-2121, or 371-2121, or a complement thereof, and encode a 3′O-kinase.

In some embodiments, as described above, the polynucleotide encodes an engineered 3′O-kinase polypeptide with improved properties as compared to SEQ ID NO: 10, wherein the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence selected from SEQ ID NO: 10, and one or more residue differences as compared to SEQ ID NO: 10, wherein the sequence is selected from the even-numbered sequences in the range of SEQ ID NOs: 56-366, or 372-2122. In some embodiments, the reference amino acid sequence is selected from the even-numbered sequences in the range of SEQ ID NOs: 56-366, or 372-2122. In some embodiments, the reference amino acid sequence is SEQ ID NO: 10, while in some other embodiments, the reference sequence is SEQ ID NO: 14.

In some embodiments, the polynucleotide encodes a 3′O-kinase polypeptide capable of converting one or more substrates to product with improved properties as compared to SEQ ID NO: 10, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 10.

In some embodiments, the polynucleotide encoding the engineered 3′O-kinase comprises a polynucleotide sequence selected from the odd-numbered sequences in the range of SEQ ID NOs: 55-365, or 371-2121, or 371-2121.

In some embodiments, the polynucleotides are capable of hybridizing under highly stringent conditions to a reference polynucleotide sequence selected from the odd-numbered sequences in the range of SEQ ID NOs: 55-365, or 371-2121, or 371-2121 or a complement thereof, and encode a 3′O-kinase polypeptide with one or more of the improved properties described herein. In some embodiments, the polynucleotide capable of hybridizing under highly stringent conditions encodes a 3′O-kinase comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 10, that has an amino acid sequence comprising one or more residue differences as compared to SEQ ID NO: 10, as described above and in the Examples, below.

In some embodiments, the polynucleotide capable of hybridizing under highly stringent conditions encodes an engineered 3′O-kinase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 10. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered 3′O-kinase. In some embodiments, the reference polynucleotide sequence is selected from SEQ ID NOs: 55-365, or 371-2121.

In some embodiments, the polynucleotide capable of hybridizing under highly stringent conditions encodes an engineered 3′O-kinase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 10. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered 3′O-kinase. In some embodiments, the reference polynucleotide sequence is selected from SEQ ID NOs: 55-365, or 371-2121.

In some embodiments, an isolated polynucleotide encoding any of the engineered 3′O-kinase polypeptides provided herein is manipulated in a variety of ways to provide for expression of the polypeptide. In some embodiments, the polynucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, suitable promoters for directing transcription of the nucleic acid constructs of the present application, include, but are not limited to the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus lichenformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Bio., 15:5983-5990 [1995]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the engineered 3′O-kinase polypeptides provided herein. Effective signal peptide coding regions for bacterial host cells include, but are not limited to the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region includes, but is not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

The present invention also provides recombinant expression vectors comprising a polynucleotide encoding an engineered 3′O-kinase polypeptide, and one or more expression regulating regions such as a promoter and a terminator, a replication origin, etc., depending on the type of hosts into which they are to be introduced. In some embodiments, the various nucleic acid and control sequences described above are combined together to produce a recombinant expression vector which includes one or more convenient restriction sites to allow for insertion or substitution of the nucleic acid sequence encoding the variant 3′O-kinase polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the variant 3′O-kinase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophy, and the like. Examples of bacterial selectable markers include, but are not limited to the dal genes from Bacillus subtilis or Bacillus lichenformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one engineered 3′O-kinase polypeptide of the present invention, the polynucleotide being operatively linked to one or more control sequences for expression of the engineered 3′O-kinase enzyme(s) in the host cell. Host cells for use in expressing the polypeptides encoded by the expression vectors of the present invention are well known in the art and include but are not limited to, bacterial cells, such as E. coli, Vibriofluvialis, Streptomyces and Salmonella typhimurium cells; fungal cells, such as yeast cells (e.g., Saccharomyces cerevisiae and Pichia pastoris [ATCC Accession No. 201178]); insect cells such as Drosophila S2 and Spodoptera Sf9 cells; animal cells such as CHO, COS, BHK, 293, and Bowes melanoma cells; and plant cells. Exemplary host cells are Escherichia coli strains (e.g., W3110 (ΔfhuA) and BL21).

In some embodiments, the host cell strain comprises a knockout of one or more genes, in particular phosphatase genes. In some embodiments, the host cell comprises a knockout or single gene deletion of E. coli genes aphA, surE, phoA, and/or cpdB, as described below in the Examples. In some embodiments, the host cell comprising a knockout of one or more phosphatase genes has increased production of the product and/or decreased de-phosphorylation of the product or substrate.

Accordingly, in another aspect, the present invention provides methods for producing the engineered 3′O-kinase polypeptides, where the methods comprise culturing a host cell capable of expressing a polynucleotide encoding the engineered 3′O-kinase polypeptide under conditions suitable for expression of the polypeptide. In some embodiments, the methods further comprise the steps of isolating and/or purifying the 3′O-kinase polypeptides, as described herein.

Appropriate culture media and growth conditions for the above-described host cells are well known in the art. Polynucleotides for expression of the 3′O-kinase polypeptides may be introduced into cells by various methods known in the art. Techniques include, among others, electroporation, biolistic particle bombardment, liposome mediated transfection, calcium chloride transfection, and protoplast fusion.

The engineered 3′O-kinases with the properties disclosed herein can be obtained by subjecting the polynucleotide encoding the naturally occurring or engineered 3′O-kinase polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, and all related US, as well as PCT and non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In some embodiments, the enzyme clones obtained following mutagenesis treatment are screened by subjecting the enzymes to a defined temperature (or other assay conditions, such as testing the enzyme's activity over a broad range of substrates) and measuring the amount of enzyme activity remaining after heat treatments or other assay conditions. Clones containing a polynucleotide encoding a 3′O-kinase polypeptide are then sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

In some embodiments, the clones obtained following mutagenesis treatment can be screened for engineered 3′O-kinases having one or more desired improved enzyme properties (e.g., improved regioselectivity). Measuring enzyme activity from the expression libraries can be performed using the standard biochemistry techniques, such as HPLC analysis, LC-MS analysis, RapidFire-MS analysis, and/or capillary electrophoresis analysis.

When the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides encoding portions of the 3′O-kinase can be prepared by chemical synthesis as known in the art (e.g., the classical phosphoramidite method of Beaucage et al., Tet. Lett. 22:1859-69 [1981], or the method described by Matthes et al., EMBO J. 3:801-05 [1984]) as typically practiced in automated synthetic methods. According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources. In some embodiments, additional variations can be created by synthesizing oligonucleotides containing deletions, insertions, and/or substitutions, and combining the oligonucleotides in various permutations to create engineered 3′O-kinases with improved properties.

Accordingly, in some embodiments, a method for preparing the engineered 3′O-kinase polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from the even-numbered sequences of SEQ ID NOs: 56-366, or 372-2122, and having one or more residue differences as compared to SEQ ID NO: 10; and (b) expressing the 3′O-kinase polypeptide encoded by the polynucleotide.

In some embodiments of the method, the polynucleotide encodes an engineered 3′O-kinase that has optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, 1-50, 1-75, 1-100, or 1-150 amino acid residue deletions, insertions and/or substitutions. In some embodiments, the amino acid sequence has optionally around 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, 50, 55, 60, 65, 70, 80, 90, 100, 110, 120, 130, 140, or 150 amino acid residue deletions, insertions and/or substitutions. In some embodiments, the substitutions can be conservative or non-conservative substitutions.

In some embodiments, any of the engineered 3′O-kinase enzymes expressed in a host cell can be recovered from the cells and/or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available (e.g., CelLytic B™, Sigma-Aldrich, St. Louis MO).

Chromatographic techniques for isolation of the 3′O-kinase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved 3′O-kinase enzymes. For affinity chromatography purification, any antibody which specifically binds the 3′O-kinase polypeptide may be used. For the production of antibodies, various host animals, including but not limited to rabbits, mice, rats, etc., may be immunized by injection with a 3′O-kinase polypeptide, or a fragment thereof. The 3′O-kinase polypeptide or fragment may be attached to a suitable carrier, such as BSA, by means of a side chain functional group or linkers attached to a side chain functional group. In some embodiments, the affinity purification can use a specific ligand bound by the 3′O-kinase or dye affinity column (See e.g., EP0641862; Stellwagen, “Dye Affinity Chromatography,” In Current Protocols in Protein Science, Unit 9.2-9.2.16 [2001]).

### Methods of Using the Engineered 3′O-Kinase Enzymes and NQP Synthesis Cascade

New methods to synthesize natural and modified NQPs are necessary to enable production of oligonucleotides on the scale required for modern synthetic biology applications in a more sustainable manner.

The present disclosure provides methods to synthesize natural and modified NQPs using one or more enzymes. In some embodiments, the present disclosure provides enzymes for the conversion of an NTP to an NTP with a phosphate group at the 3′ position of the sugar (NQP), as depicted in Schemes 1, 3, and 4, above.

In some embodiments, the present disclosure provides enzymes for the conversion of a nucleoside to an NMP via addition of a phosphate group to the 5′ position of the sugar. In some embodiments, the present invention provides enzymes for the conversion of an NMP to an NDP. In some embodiments, the present disclosure provides enzymes for the conversion of an NDP to an NTP. In some embodiments, the present disclosure provides enzymes for the conversion of an NTP to an NQP. In some embodiments, the present disclosure provides a one-pot method for conversion of nucleosides to NQPs. In some embodiments, the present disclosure provides a one-pot method, two step method for conversion of nucleosides to NQPs.

In any of the above embodiments, the nucleosides, NMPs, NDPs, NTPs, NQPs, p4A, and other byproduct species may be natural or may comprise one or more modifications. In any of the above embodiments, the nucleosides, NMPs, NDPs, NTPs, NQPs, p4A, and other byproduct species may comprise ribonucleosides, deoxyribonucleosides, dideoxynucleosides, or modified nucleosides. In any of the above embodiments, the nucleosides, NMPs, NDPs, NTPs, NQPs, p4A, and other byproduct species may comprise one or more modifications to the sugar. In any of the above embodiments, the nucleosides, NMPs, NDPs, NTPs, NQPs, p4A, and other byproduct species may comprise one or more modifications to the nucleobase. In any of the above embodiments, the NMPs, NDPs, NTPs, NQPs, p4A, and other byproduct species may comprise an α-thiophosphate or dithiophosphate or other modification to the 5′ phosphate chain. Any of these modifications may be present in any combination in the 3′O-kinase substrate or may be added after or during conversion to the 3′O-kinase product.

In some embodiments, the 2′-R group of the sugar comprises H, OH, OCH₃, OCH₂CH₂OCH₃, F, CO₂R′ (where R′ is any alkyl or aryl), or another atom or chemical group. In some embodiments, the sugar may have other modifications at other positions, such as locked nucleotides or constrained ethyl nucleotides, as is known in the art. In some embodiments, “locked nucleoside” or “locked nucleotide” refers to nucleoside or nucleotide, respectively, in which the ribose moiety is modified with a bridge connecting the 2′ oxygen and 4′ carbon (see, e.g., Obika et al., Tetrahedron Letters, 1997, 38(50):8735-8738; Orum et al., Current Pharmaceutical Design, 2008, 14(11):1138-1142). Typically, the bridge is a methylene bridge. In some embodiments, the 3′-phosphate group of the NQP may act as a removable blocking group or protecting group that may be selectively unblocked or removed to allow further modifications, reactions, or incorporation of the NQP into a growing oligonucleotide chain during template-dependent or template-independent oligonucleotide synthesis.

In some embodiments, the nucleobase may be a uridine, thymine, cytosine, adenine, guanine or another nucleobase known to those skilled in the art. In some embodiments, the nucleobase of the nucleoside, NMP, NTP, NDP, NTP, NQP, p4A, or byproduct species may have modifications. Various modified nucleobases are known to those skilled in the art, including but not limited to the following: 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, propyne, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethy 1-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methyl-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N7-methyl-guanine. In some embodiments, the nucleobase modification is a removable tag, a cleavable linker, or a radio, photo, or chemical sensor. In some embodiments, the nucleobase modification is a functional element that may be used for isolation, purification, detection, protection, prevention of hydrolysis or degradation, chemical transformation, or to enable further or sequential modifications.

In some embodiments, the NMP, NTP, NDP, NTP, NQP, p4A, or byproduct species comprises one or more modifications to the 5′ phosphate chain. The 5′ phosphate chain may comprise one, two, or three phosphates or no phosphates may be present. The 5′ phosphate chain may also comprise one or more phosphate groups with modifications (e.g. an α-thiophosphate or dithiophosphate).

In particular, the engineered 3′O-kinase polypeptides of the present disclosure have been engineered for efficient synthesis of NQPs, in the processes depicted in Scheme 1, 3, and 4, above. A variety of suitable reaction conditions are known to those skilled in the art, including the reaction conditions detailed in the Examples. A variety of methods of generating NQPs are possible using the enzymes, substrates, and cofactors described herein. These embodiments are intended to be non-limiting; the present disclosure contemplates methods comprising every combination of enzymes, substrates, and cofactors described herein.

In some embodiments, the present disclosure provides a method of producing an NTP with a phosphate group at the 3′ position of the sugar or NQP, the method comprising (i) providing a 3′O-kinase enzyme, and (ii) contacting the 3′O-kinase enzyme with an NTP under suitable reaction conditions, such that an NTP with a phosphate group at the 3′ position of the sugar or NQP is produced. In certain embodiments, the method comprises (i) providing an engineered 3′O-kinase comprising one or more amino acid residue substitutions as compared to a wild type or reference polypeptide, and (ii) contacting the engineered 3′O-kinase enzyme with an NTP under suitable reaction conditions, such that an NTP with a phosphate group at the 3′ position of the sugar or an NQP is produced. In certain embodiments, the method comprises (i) providing a 3′O-kinase enzyme, and (ii) contacting the 3′O-kinase enzyme with an NTP with one or modifications under suitable reaction conditions, such that an NTP with a phosphate group at the 3′ position of the sugar or NQP with one or more modifications is produced. In certain embodiments, the method comprises (i) providing an engineered 3′O-kinase comprising one or more amino acid residue substitutions as compared to a wild type or reference polypeptide, and (ii) contacting the engineered 3′O-kinase enzyme with an NTP, optionally, with one or modifications, under suitable reaction conditions, such that an NTP with a phosphate group at the 3′ position of the sugar or NQP, optionally, with one or modifications, is produced. In any of the above embodiments, the method may further comprise providing a source of phosphate, and, optionally, one or more phosphate recycling enzymes including but not limited to pyruvate oxidase and/or acetate kinase. In any of the above embodiments, the method may further comprise providing more than one 3′O-kinase in step (i). In any of the above embodiments, the method may further comprise a 3′O-kinase comprising increased activity, increased activity on non-natural substrates, increased selectivity, increased substrate promiscuity, decreased product inhibition, and/or decreased byproduct formation, as compared to a wild-type or reference 3′O-kinase. In any of the above embodiments, the method may further comprise a phosphate donor NTP that is the same type of NTP as the substrate NTP. In any of the above embodiments, the method may further comprise a phosphate donor NTP that is a different type of NTP than the substrate NTP.

In certain embodiments, the present disclosure provides a method of producing an NTP with a phosphate group at the 3′ position of the sugar or NQP, the method comprising (i) providing an engineered 3′O-kinase comprising at least 60%, 70%, 80%, 90%, or 95% sequence identity to one or more of the even-numbered sequences between SEQ ID NO: 56-366, or 372-2122, and (ii) contacting the engineered 3′O-kinase enzyme with an NTP, optionally, with one or modifications, under suitable reaction conditions, such that an NTP with a phosphate group at the 3′ position of the sugar or NQP, optionally, with one or modifications, is produced. In some embodiments, the method comprises (i) providing an engineered 3′O-kinase comprising at least 60%, 70%, 80%, 90%, or 95% sequence identity to one or more of the even-numbered sequences between SEQ ID NO: 56-366, or 372-2122, and (ii) contacting the engineered 3′O-kinase enzyme with an NTP, optionally, with one or modifications, under suitable reaction conditions, such that an NTP with a phosphate group at the 3′ position of the sugar or NQP, optionally, with one or modifications, is produced, wherein said engineered 3′O-kinase converts said NTP to NQP with a conversion rate that is at least 1.5 fold, 2 fold, 5 fold, 10 fold or more increased, as compared to a wild type or reference 3′O-kinase. In certain embodiments, the method comprises (i) providing an engineered 3′O-kinase comprising at least 60%, 70%, 80%, 90%, or 95% sequence identity to one or more of the even-numbered sequences between SEQ ID NO: 56-366, or 372-2122, and (ii) contacting the engineered 3′O-kinase enzyme with an NTP, optionally, with one or modifications, under suitable reaction conditions, such that an NTP with a phosphate group at the 3′ position of the sugar or NQP, optionally, with one or modifications, is produced, wherein said engineered 3′O-kinase converts said NTP to NQP with a selectivity for NQP over NPP that is at least 1.1 fold, 1.2 fold, 1.5 fold, 2 fold, 5 fold or more increased, as compared to a wild type or reference 3′O-kinase. In any of the above embodiments, the method may further comprise providing a source of phosphate, and, optionally, one or more phosphate recycling enzymes including but not limited to pyruvate oxidase and/or acetate kinase. In any of the above embodiments, the method may further comprise providing more than one 3′O-kinase in step (i). In any of the above embodiments, the method may further comprise a 3′O-kinase comprising increased activity, increased activity on non-natural substrates, increased selectivity, increased substrate promiscuity, decreased product inhibition, and/or decreased byproduct formation, as compared to a wild-type or reference 3′O-kinase. In any of the above embodiments, the method may further comprise a phosphate donor NTP that is the same type of NTP as the substrate NTP. In any of the above embodiments, the method may further comprise a phosphate donor NTP that is a different type of NTP than the substrate NTP. In any of the above embodiments, the 3′O-kinase may comprise any of the polypeptides encoded by the even-numbered sequences from SEQ ID NO: 56-366, or 372-2122.

In some embodiments, the present disclosure provides a one-pot method for conversion of nucleosides to NQPs, as depicted in Scheme 3, above. In some embodiments, the one-pot method for conversion of nucleosides to NQPs occurs in one step.

In some embodiments, the present disclosure provides a method of producing an NTP with a phosphate group at the 3′ position of the sugar or NQP, the method comprising (i) providing a 5′O-kinase enzyme, a nucleoside monophosphate kinase enzyme, an acetate kinase enzyme, and a 3′O-kinase enzyme, and (ii) contacting the 5′O-kinase enzyme, the nucleoside monophosphate kinase enzyme, the acetate kinase enzyme, and the 3′O-kinase enzyme with a nucleoside under suitable reaction conditions, such that an NTP with a phosphate group at the 3′ position of the sugar or NQP is produced. In certain embodiments, the method comprises (i) providing a 5′O-kinase enzyme, a nucleoside monophosphate kinase enzyme, an acetate kinase enzyme, and an engineered 3′O-kinase comprising one or more amino acid residue substitutions as compared to a wild type or reference polypeptide, and (ii) contacting the 5′O-kinase enzyme, the nucleoside monophosphate kinase enzyme, the acetate kinase enzyme, and the engineered 3′O-kinase enzyme with a nucleoside under suitable reaction conditions, such that an NTP with a phosphate group at the 3′ position of the sugar or NQP is produced. In certain embodiments, the method comprises (i) providing a 5′O-kinase enzyme, a nucleoside monophosphate kinase enzyme, an acetate kinase enzyme, and a 3′O-kinase enzyme and (ii) contacting the 5′O-kinase enzyme, the nucleoside monophosphate kinase enzyme, the acetate kinase enzyme and the 3′O-kinase enzyme with a nucleoside with one or modifications under suitable reaction conditions, such that an NTP with a phosphate group at the 3′ position of the sugar or NQP with one or more modifications is produced. In certain embodiments, the method comprises (i) providing a 5′O-kinase enzyme, a nucleoside monophosphate kinase enzyme, an acetate kinase enzyme, and an engineered 3′O-kinase comprising one or more amino acid residue substitutions as compared to a wild type or reference polypeptide, and (ii) contacting the 5′O-kinase enzyme, the nucleoside monophosphate kinase enzyme, the acetate kinase enzyme, and the engineered 3′O-kinase enzyme with a nucleoside with one or modifications under suitable reaction conditions, such that an NTP with a phosphate group at the 3′ position of the sugar or NQP with one or more modifications is produced. In certain embodiments, the method comprises (i) providing a 5′O-kinase enzyme, a nucleoside monophosphate kinase enzyme, an acetate kinase enzyme, and an engineered 3′O-kinase comprising at least 60%, 70%, 80%, 90%, or 95% sequence identity to one or more of the even-numbered sequences between SEQ ID NO: 56-366, or 372-2122, and (ii) contacting the 5′O-kinase enzyme, the nucleoside monophosphate kinase enzyme, the acetate kinase enzyme, and the engineered 3′O-kinase enzyme with a nucleoside with one or modifications under suitable reaction conditions, such that an NTP with a phosphate group at the 3′ position of the sugar or NQP with one or more modifications is produced. In any of the above embodiments, the method may further comprise providing a source of phosphate, and, optionally, one or more phosphate recycling enzymes, including but not limited to pyruvate oxidase and/or acetate kinase. In any of the above embodiments, the method may further comprise providing more than one 3′O-kinase in step (i). In any of the above embodiments, the method may further comprise a 3′O-kinase comprising increased activity, increased activity on non-natural substrates, increased selectivity, increased substrate promiscuity, decreased product inhibition, and/or decreased byproduct formation, as compared to a wild-type or reference 3′O-kinase. In any of the above embodiments, the method may further comprise a phosphate donor NTP that is the same type of NTP as the substrate NTP. In any of the above embodiments, the method may further comprise a phosphate donor NTP that is a different type of NTP than the substrate NTP. In any of the above embodiments, the 3′O-kinase may comprise any of the polypeptides encoded by the even-numbered sequences from SEQ ID NO: 56-366, or 372-2122.

In some embodiments, the present disclosure provides a one-pot method for conversion of nucleosides to NQPs. In some embodiments, the one-pot method for conversion of nucleosides to NQPs occurs in more than one step. In some embodiments, the one-pot method for conversion of nucleosides to NQPs occurs in two steps, wherein the second step is depicted in Scheme 1, above, and the first step is depicted in Scheme 4, above.

In some embodiments, the present disclosure provides a method of producing an NTP with a phosphate group at the 3′ position of the sugar or NQP, the method comprising (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside under suitable reaction conditions, such that an NTP is produced; iii) providing a 3′O-kinase enzyme; and (iv) contacting the 3′O-kinase enzyme with the NTP under suitable reaction conditions, such that an NTP with a phosphate group at the 3′ position of the sugar or NQP is produced. In certain embodiments, the method comprises (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside under suitable reaction conditions, such that an NTP is produced; iii) providing an engineered 3′O-kinase comprising one or more amino acid residue substitutions as compared to a wild type or reference polypeptide; and (iv) contacting the engineered 3′O-kinase enzyme with the NTP under suitable reaction conditions, such that an NTP with a phosphate group at the 3′ position of the sugar or NQP is produced. In certain embodiments, the method comprises (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside with one or more modifications under suitable reaction conditions, such that an NTP with one or more modifications is produced; iii) providing a 3′O-kinase enzyme; and (iv) contacting the 3′O-kinase enzyme with the NTP with one or more modifications under suitable reaction conditions, such that an NTP with a phosphate group at the 3′ position of the sugar or NQP with one or more modifications is produced. In certain embodiments, the method comprises (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside with one or more modifications under suitable reaction conditions, such that an NTP with one or more modifications is produced; iii) providing an engineered 3′O-kinase comprising one or more amino acid residue substitutions as compared to a wild type or reference polypeptide; and (iv) contacting the engineered 3′O-kinase enzyme with the NTP with one or more modifications under suitable reaction conditions, such that an NTP with a phosphate group at the 3′ position of the sugar or NQP with one or more modifications is produced. In any of the above embodiments, the method may further comprise providing a source of phosphate, and, optionally, one or more phosphate recycling enzymes, including but not limited to pyruvate oxidase and/or acetate kinase. In any of the above embodiments, the method may further comprise a 3′O-kinase comprising increased activity, increased activity on non-natural substrates, increased selectivity, increased substrate promiscuity, decreased product inhibition, and/or decreased byproduct formation, as compared to a wild-type or reference 3′O-kinase. In any of the above embodiments, the method may further comprise providing more than one 3′O-kinase in step (i). In any of the above embodiments, the method may further comprise a phosphate donor NTP that is the same type of NTP as the substrate NTP. In any of the above embodiments, the method may further comprise a phosphate donor NTP that is a different type of NTP than the substrate NTP.

In some embodiments, the present disclosure provides a method of producing an NTP with a phosphate group at the 3′ position of the sugar or NQP, the method comprising (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside, optionally, with one or modifications, under suitable reaction conditions, such that an NTP, optionally, with one or modifications, is produced; iii) providing an engineered 3′O-kinase comprising at least 60%, 70%, 80%, 90%, or 95% sequence identity to one or more of the even-numbered sequences between SEQ ID NO: 56-366, or 372-2122; and (iv) contacting the engineered 3′O-kinase enzyme with the NTP, optionally, with one or modifications, under suitable reaction conditions, such that an NTP with a phosphate group at the 3′ position of the sugar or NQP, optionally, with one or modifications, is produced. In some embodiments, the method comprises (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside, optionally, with one or modifications, under suitable reaction conditions, such that an NTP, optionally, with one or modifications, is produced; iii) providing an engineered 3′O-kinase comprising at least 60%, 70%, 80%, 90%, or 95% sequence identity to one or more of the even-numbered sequences between SEQ ID NO: 56-366, or 372-2122; and (iv) contacting the engineered 3′O-kinase enzyme with the NTP, optionally, with one or modifications, under suitable reaction conditions, such that an NTP with a phosphate group at the 3′ position of the sugar or NQP, optionally, with one or modifications, is produced, wherein said engineered 3′O-kinase converts said NTP to NQP with a conversion rate that is at least 1.5 fold, 2 fold, 5 fold, 10 fold or more increased, as compared to a wild type or reference 3′O-kinase. In some embodiments, the method comprises (i) providing a 5′O-kinase enzyme, a nucleoside monophosphate kinase enzyme, and an acetate kinase enzyme; (ii) contacting the 5′O-kinase enzyme, the nucleoside monophosphate kinase enzyme, and the acetate kinase enzyme with a nucleoside, optionally, with one or modifications, under suitable reaction conditions, such that an NTP, optionally, with one or modifications, is produced; iii) providing an engineered 3′O-kinase comprising at least 60%, 70%, 80%, 90%, or 95% sequence identity to one or more of the even-numbered sequences between SEQ ID NO: 56-366, or 372-2122; and (iv) contacting the engineered 3′O-kinase enzyme with the NTP, optionally, with one or modifications, under suitable reaction conditions, such that an NTP with a phosphate group at the 3′ position of the sugar or NQP, optionally, with one or modifications, is produced, wherein said engineered 3′O-kinase converts said NTP to NQP with a selectivity for NQP over NPP that is at least 1.1 fold, 1.2 fold, 1.5 fold, 2 fold, 5 fold or more increased, as compared to a wild type or reference 3′O-kinase. In any of the above embodiments, the method may further comprise providing a source of phosphate, and, optionally, one or more phosphate recycling enzymes, including but not limited to pyruvate oxidase and/or acetate kinase. In any of the above embodiments, the method may further comprise a 3′O-kinase comprising increased activity, increased activity on non-natural substrates, increased selectivity, increased substrate promiscuity, decreased product inhibition, and/or decreased byproduct formation, as compared to a wild-type or reference 3′O-kinase. In any of the above embodiments, the method may further comprise providing more than one 3′O-kinase in step (i). In any of the above embodiments, the method may further comprise a phosphate donor NTP that is the same type of NTP as the substrate NTP. In any of the above embodiments, the method may further comprise a phosphate donor NTP that is a different type of NTP than the substrate NTP. In any of the above embodiments, the 3′O-kinase may comprise any of the polypeptides encoded by the even-numbered sequences from SEQ ID NO: 56-366, or 372-2122.

In any of the disclosed embodiments, the nucleosides, NMPs, NDPs, NTPs, NQPs, p4A and other byproduct species may be natural or may comprise one or more modifications. In any of the disclosed embodiments, the nucleosides, NMPs, NDPs, NTPs, NQPs, p4A and other byproduct species may comprise ribonucleosides, deoxyribonucleosides, dideoxynucleosides, or modified nucleosides. In any of the disclosed embodiments, the nucleosides, NMPs, NDPs, NTPs, NQPs, p4A and other byproduct species may comprise one or more modifications to the sugar. In any of the disclosed embodiments, the nucleosides, NMPs, NDPs, NTPs, NQPs, p4A and other byproduct species may comprise one or more modifications to the nucleobase. In any of the disclosed embodiments, the nucleosides, NMPs, NDPs, NTPs, NQPs, p4A and other byproduct species may comprise an α-thiophosphate or dithiophosphate or other modification to the 5′ phosphate chain.

In some embodiments, the enzymes described herein find use in processes for conversion of one or more suitable substrates to a product.

In some embodiments, the engineered 3′O-kinase polypeptides disclosed herein can be used in a process for the conversion of a natural or modified nucleoside, NMP, NDP, or NTP substrate to a product comprising a natural or modified nucleoside, NMP, NDP, or NTP with a phosphate group at the 3′ position of the sugar.

In the embodiments provided herein and illustrated in the Examples, various ranges of suitable reaction conditions that can be used in the processes, include but are not limited to, substrate loading, co-substrate loading, pH, temperature, buffer, solvent system, cofactor, polypeptide loading, and reaction time. Further suitable reaction conditions for carrying out the process for biocatalytic conversion of substrate compounds to product compounds using the enzymes described herein can be readily optimized in view of the guidance provided herein by routine experimentation that includes, but is not limited to, contacting the enzymes and one or more substrate compounds under experimental reaction conditions of concentration, pH, temperature, and solvent conditions, and detecting the product compound. The reaction conditions described herein are examples only. The present disclosure contemplates any suitable reaction conditions that may find use in the methods described herein.

The substrate compound in the reaction mixtures can be varied, taking into consideration, for example, the desired amount of product compound, the effect of each substrate concentration on enzyme activity, stability of enzyme under reaction conditions, and the percent conversion of each substrate to product. In some embodiments, the suitable reaction conditions comprise a substrate compound loading of at least about 0.1 uM to 1 uM, 1 uM to 2 uM, 2 uM to 3 uM, 3 uM to 5 uM, 5 uM to 10 uM, or 10 uM or greater. In some embodiments, the suitable reaction conditions comprise a substrate compound loading of at least about 0.5 to about 25 g/L, 1 to about 25 g/L, 5 to about 25 g/L, about 10 to about 25 g/L, or 20 to about 25 g/L. In some embodiments, the suitable reaction conditions comprise a substrate compound loading of at least about 0.5 g/L, at least about 1 g/L, at least about 5 g/L, at least about 10 g/L, at least about 15 g/L, at least about 20 g/L, or at least about 30 g/L, or even greater.

In carrying out the synthesis processes described herein, the engineered polypeptides may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the enzyme(s) or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

The gene(s) encoding the polypeptides can be transformed into host cell separately or together into the same host cell. For example, in some embodiments one set of host cells can be transformed with gene(s) encoding one polypeptide and another set can be transformed with gene(s) encoding another polypeptide. Both sets of transformed cells can be utilized together in the reaction mixture in the form of whole cells, or in the form of lysates or extracts derived therefrom. In other embodiments, a host cell can be transformed with gene(s) encoding multiple polypeptides. In some embodiments the polypeptides can be expressed in the form of secreted polypeptides and the culture medium containing the secreted polypeptides can be used for the synthesis reaction.

In some embodiments, the improved activity of the engineered 3′O-kinase polypeptides disclosed herein provides for processes wherein higher percentage conversion can be achieved with lower concentrations of the engineered polypeptide. In some embodiments of the process, the suitable reaction conditions comprise an engineered polypeptide amount of about 1% (w/w), 2% (w/w), 5% (w/w), 10% (w/w), 20% (w/w), 30% (w/w), 40% (w/w), 50% (w/w), 75% (w/w), 100% (w/w) or more of substrate compound loading.

In some embodiments, the engineered polypeptide is present at a molar ratio of engineered polypeptide to substrate of about 50 to 1, 25 to 1, 10 to 1, 5 to 1, 1 to 1, 1 to 5, 1 to 10, 1 to 25 or 1 to 50. In some embodiments, the engineered polypeptide is present at a molar ratio of engineered polypeptide to substrate from a range of about 50 to 1 to a range of about 1 to 50.

In some embodiments, the engineered polypeptide is present at about 0.01 g/L to about 50 g/L; about 0.01 to about 0.1 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the 3′O-kinase polypeptide is present at about 0.01 g/L, 0.05 g/L, 0.1 g/L, 0.2 g/L, 0.5 g/L, 1, 2 g/L, 5 g/L, 10 g/L, 15 g/L, 20 g/L, 25 g/L, 30 g/L, 35 g/L, 40 g/L, or 50 g/L.

In some embodiments, the suitable reaction conditions comprise a phosphate donor. In some embodiments, the phosphate donor is an NTP. In some embodiments, the phosphate donor is acetyl phosphate. In some embodiments, the phosphate donor is present at concentrations of about 1 to 500 uM; about 50 to 400 uM; about 100 to 300 uM; or about 200 to 300 uM. In some embodiments, the phosphate donor is regenerated or created by an enzyme, so that a lower concentration of phosphate donor is used.

During the course of the reaction, the pH of the reaction mixture may change. The pH of the reaction mixture may be maintained at a desired pH or within a desired pH range. This may be done by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, borate, potassium phosphate, 2-(N-morpholino)ethanesulfonic acid (MES), 3-(N-morpholino)propanesulfonic acid (MOPS), acetate, triethanolamine, and 2-amino-2-hydroxymethyl-propane-1,3-diol (Tris), and the like. In some embodiments, the reaction conditions comprise water as a suitable solvent with no buffer present.

In the embodiments of the process, the reaction conditions comprise a suitable pH. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In the embodiments of the processes herein, a suitable temperature is used for the reaction conditions, for example, taking into consideration the increase in reaction rate at higher temperatures, and the activity of the enzyme during the reaction time period. Accordingly, in some embodiments, the suitable reaction conditions comprise a temperature of about 10° C. to about 95° C., about 10° C. to about 75° C., about 15° C. to about 95° C., about 20° C. to about 95° C., about 20° C. to about 65° C., about 25° C. to about 70° C., or about 50° C. to about 70° C. In some embodiments, the suitable reaction conditions comprise a temperature of about 10° C., 15° C., 20° C., 25° C., 30° C., 35° C., 40° C., 45° C., 50° C., 55° C., 60° C., 65° C., 70° C., 75° C., 80° C., 85° C., 90° C. or 95° C. In some embodiments, the temperature during the enzymatic reaction can be maintained at a specific temperature throughout the course of the reaction. In some embodiments, the temperature during the enzymatic reaction can be adjusted over a temperature profile during the course of the reaction.

In some embodiments, the processes of the invention are carried out in a solvent. Suitable solvents include water, aqueous buffer solutions, organic solvents, polymeric solvents, and/or co-solvent systems, which generally comprise aqueous solvents, organic solvents and/or polymeric solvents. The aqueous solvent (water or aqueous co-solvent system) may be pH-buffered or unbuffered. In some embodiments, the processes using the engineered 3′O-kinase polypeptides can be carried out in an aqueous co-solvent system comprising an organic solvent (e.g., ethanol, isopropanol (IPA), dimethyl sulfoxide (DMSO), dimethylformamide (DMF) ethyl acetate, butyl acetate, 1-octanol, heptane, octane, methyl t butyl ether (MTBE), toluene, and the like), ionic or polar solvents (e.g., 1-ethyl 4 methylimidazolium tetrafluoroborate, 1-butyl-3-methylimidazolium tetrafluoroborate, 1-butyl 3 methylimidazolium hexafluorophosphate, glycerol, polyethylene glycol, and the like). In some embodiments, the co-solvent can be a polar solvent, such as a polyol, dimethylsulfoxide (DMSO), or lower alcohol. The non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the enzymes under the reaction conditions. Appropriate co-solvent systems can be readily identified by measuring the enzymatic activity of the specified enzymes with a defined substrate of interest in the candidate solvent system, utilizing an enzyme activity assay, such as those described herein.

In some embodiments of the process, the suitable reaction conditions comprise an aqueous co-solvent, where the co-solvent comprises DMSO at about 1% to about 50% (v/v), about 1 to about 40% (v/v), about 2% to about 40% (v/v), about 5% to about 30% (v/v), about 10% to about 30% (v/v), or about 10% to about 20% (v/v). In some embodiments of the process, the suitable reaction conditions can comprise an aqueous co-solvent comprising ethanol at about 1% (v/v), about 5% (v/v), about 10% (v/v), about 15% (v/v), about 20% (v/v), about 25% (v/v), about 30% (v/v), about 35% (v/v), about 40% (v/v), about 45% (v/v), or about 50% (v/v).

In some embodiments, the reaction conditions comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), TRITON™ X-100 polyethylene glycol tert-octylphenyl ether, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/ml, particularly from 1 to 20 mg/ml.

In some embodiments, the reaction conditions include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include Y-30® (Dow Corning), poly-glycol copolymers, oxy/ethoxylated alcohols, and polydimethylsiloxanes. In some embodiments, the anti-foam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the anti-foam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

The quantities of reactants used in the synthesis reaction will generally vary depending on the quantities of product desired, and concomitantly the amount of substrates employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor, co-substrate and substrate may be added first to the solvent.

The solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

For improved mixing efficiency when an aqueous co-solvent system is used, the polypeptide(s), and co-substrate may be added and mixed into the aqueous phase first. The substrate may be added and mixed in, followed by the organic phase or the substrate may be dissolved in the organic phase and mixed in. Alternatively, the substrate may be premixed in the organic phase, prior to addition to the aqueous phase.

The processes of the present invention are generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like. In some embodiments, after suitable conversion to product, the reactants are separated from the product and additional reactants are added.

Any of the processes disclosed herein using the polypeptides for the preparation of products can be carried out under a range of suitable reaction conditions, including but not limited to ranges of substrates, temperature, pH, solvent system, substrate loading, polypeptide loading, cofactor loading, and reaction time. In one example, the suitable reaction conditions for the conversion of an NTP to an NQP comprise: (a) substrate loading of about 1-200 mM NTP; (b) about 0.01 g/L to 5 g/L engineered 3′O-kinase polypeptide; (c) 1-100 mM MgCl₂; (e) 5 to 100 mM tris-HCl buffer; (f) 10-100 mM LiKAcPO₄⁻; (g) pH at 5-9; and (h) temperature of about 15° C. to 70° C. In one example, the suitable reaction conditions for the conversion of an NTP to an NQP comprise: (a) substrate loading of about 50 mM NTP; (b) about 0.01 g/L to 5 g/L engineered 3′O-kinase polypeptide; (c) 10 mM MgCl₂; (e) 50 mM tris-HCl buffer; (f) 10 mM LiKAcPO₄⁻; (g) pH 7.5; and (h) temperature of about 25° C. In some embodiments, the enzyme loading is between 1-30% w/w. In some embodiments, additional reaction components or additional techniques carried out to supplement the reaction conditions. These can include taking measures to stabilize or prevent inactivation of the enzyme, reduce product inhibition, shift reaction equilibrium to formation of the desired product.

Accordingly, it is further contemplated that any of the methods of using the polypeptides of the present invention can be carried out using the polypeptides bound or immobilized on a solid support.

Methods of enzyme immobilization are well-known in the art. The engineered polypeptides can be bound non-covalently or covalently. Various methods for conjugation and immobilization of enzymes to solid supports (e.g., resins, membranes, beads, glass, etc.) are well known in the art (See e.g., Yi et al., Proc. Biochem., 42(5): 895-898 [2007]; Martin et al., Appl. Microbiol. Biotechnol., 76(4): 843-851 [2007]; Koszelewski et al., J. Mol. Cat. B: Enzymatic, 63: 39-44 [2010]; Truppo et al., Org. Proc. Res. Dev., published online: dx.doi.org/10.1021/op200157c; Hermanson, Bioconjugate Techniques, 2^{nd} ed., Academic Press, Cambridge, MA [2008]; Mateo et al., Biotechnol. Prog., 18(3):629-34 [2002]; and “Bioconjugation Protocols: Strategies and Methods,” In Methods in Molecular Biology, Niemeyer (ed.), Humana Press, New York, NY [2004]; the disclosures of each which are incorporated by reference herein). Solid supports useful for immobilizing the engineered 3′O-kinase of the present invention include but are not limited to beads or resins comprising polymethacrylate with epoxide functional groups, polymethacrylate with amino epoxide functional groups, styrene/DVB copolymer or polymethacrylate with octadecyl functional groups. Exemplary solid supports useful for immobilizing the engineered 3′O-kinase polypeptides of the present invention include, but are not limited to, EnginZyme (including, EziG-1, EziG-1, and EziG-3), chitosan beads, Eupergit C, and SEPABEADs (Mitsubishi) (including EC-EP, EC-HFA/S, EXA252, EXE119 and EXE120).

In further embodiments, any of the above described processes for the conversion of one or more substrate compounds to product compound can further comprise one or more steps selected from: extraction; isolation; purification; and crystallization of product compound. Methods, techniques, and protocols for extracting, isolating, purifying, and/or crystallizing the product from biocatalytic reaction mixtures produced by the above disclosed processes are known to the ordinary artisan and/or accessed through routine experimentation. Additionally, illustrative methods are provided in the Examples below.

Various features and embodiments of the invention are illustrated in the following representative examples, which are intended to be illustrative, and not limiting.

### EXAMPLES

The following Examples, including experiments and results achieved, are provided for illustrative purposes only and are not to be construed as limiting the present invention. Indeed, there are various suitable sources for many of the reagents and equipment described below. It is not intended that the present invention be limited to any particular source for any reagent or equipment item.

In the experimental disclosure below, the following abbreviations apply: M (molar); mM (millimolar), uM and μM (micromolar); nM (nanomolar); mol (moles); gm and g (gram); mg (milligrams); ug and μg (micrograms); L and 1 (liter); ml and mL (milliliter); cm (centimeters); mm (millimeters); um and μιη(micrometers); sec. (seconds); min(s) (minute(s)); h(s) and hr(s) (hour(s)); U (units); MW (molecular weight); rpm (rotations per minute); psi and PSI (pounds per square inch); ° C. (degrees Celius); RT and rt (room temperature); CV (coefficient of variability); CAM and cam (chloramphenicol); PMBS (polymyxin B sulfate); IPTG (isopropyl β-D-1-thiogalactopyranoside); LB (lysogeny broth); TB (terrific broth); SFP (shake flask powder); CDS (coding sequence); DNA (deoxyribonucleic acid); RNA (ribonucleic acid); nt (nucleotide; polynucleotide); aa (amino acid; polypeptide); E. coli W3110 (commonly used laboratory E. coli strain, available from the Coli Genetic Stock Center [CGSC], New Haven, CT); HTP (high throughput); HPLC (high pressure liquid chromatography); HPLC-UV (HPLC-Ultraviolet Visible Detector); 1H NMR (proton nuclear magnetic resonance spectroscopy); FIOPC (fold improvements over positive control); Sigma and Sigma-Aldrich (Sigma-Aldrich, St. Louis, MO; Difco (Difco Laboratories, BD Diagnostic Systems, Detroit, MI); Microfluidics (Microfluidics, Westwood, MA); Life Technologies (Life Technologies, a part of Fisher Scientific, Waltham, MA); Amresco (Amresco, LLC, Solon, OH); Carbosynth (Carbosynth, Ltd., Berkshire, UK); Varian (Varian Medical Systems, Palo Alto, CA); Agilent (Agilent Technologies, Inc., Santa Clara, CA); Infors (Infors USA Inc., Annapolis Junction, MD); and Thermotron (Thermotron, Inc., Holland, MI).

### Example 1

### Gene Acquisition and Expression of Wild-Type 3OK Variants

Synthetic genes encoding an N-terminal or C-terminal 6-histidine tagged version of multiple wild-type (WT) 3′O-Kinase (30K) enzymes were cloned into the pCK110900 vector system (See e.g., U.S. Pat. No. 9,714,437, which is hereby incorporated by reference in its entirety) and subsequently expressed in an E. coli strain derived from W3110.

Cells transformed with the 3OK expression constructs were grown at shake-flask scale using either IPTG induction (SEQ ID NOs: 3 and 5) or auto-induction (SEQ ID NOs: 1, 7-15), as described in Example 7, (Methods 1 and 2 respectively). Cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and 3OK concentrations were measured by absorption at 280 nm using a NanoDrop™ 1000 spectrophotometer. Soluble protein concentrations are summarized in Table 1.1 below, showing a fold improvement in soluble protein production following shake-flask purification relative to the 3′O-kinase from Thermosynechococcus vestitus (SEQ ID NO: 2).

TABLE 1.1
Soluble Enzyme Production of Variants Relative to SEQ ID NO: 2

| SEQ ID NO: | | FIOP Soluble Enzyme Production |
|---|---|---|
| (nt/aa) | Source organism of 3OK gene sequence | (Relative to SEQ ID NO: 2) |
| 3/4 | E coli W3110 | +++ |
| 5/6 | E coli W3110 | +++ |
| 7/8 | Thermomonas hydrothermalis | +++ |
| 9/10 | Geobacillus stearothermophilus | ++ |
| 11/12 | Aquifex aeolicus | ++ |
| 13/14 | Thermotoga sp. RQ7 | ++ |
| 15/16 | Caldibacillus thermoamylovorans | + |
| 1/2 | Thermosynechococcus vestitus | + |

Levels of increased soluble enzyme production were determined relative to the reference polypeptide of SEQ ID NO: 2 and defined as follows:
“+” 1.00 to 6.40,
“++” >6.40,
“+++” >21.60

### Example 2

### Gene Acquisition and Expression of Adenylate Kinase Variants

Synthetic genes encoding N-terminal 6-histidine tagged versions of wild-type (WT) and evolved adenylate kinase enzymes (AdK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the adenylate kinase expression construct were grown at shake-flask scale, as described in Example 7, (Method 1). Cells were then lysed, purified, and dialyzed into storage buffer (20 mM Tris-HCl, pH 7.4, 100 mM KCl, 0.1 mM EDTA, and 50% glycerol), as described in Example 7. After overnight dialysis, protein samples were removed, and adenylate kinase enzyme concentrations were measured by absorption at 280 nm using a NanoDrop™ 1000 spectrophotometer. Soluble protein concentrations are summarized in Table 2.1 below.

TABLE 2.1
Soluble Enzyme Production of Variants Relative to SEQ ID NO: 20

| SEQ ID NO: | | FIOP Soluble Enzyme Production |
|---|---|---|
| (nt/aa) | Source organism of AdK gene sequence | (Relative to SEQ ID NO: 20) |
| 21/22 | Saccharomyces cerevisiae | +++ |
| 23/24 | Saccharomyces cerevisiae | +++ |
| 25/26 | Thermotoga neapolitana | ++ |
| 27/28 | Escherichia coli | ++ |
| 19/20 | Geobacillus stearothermophilus | + |

Levels of increased soluble enzyme production were determined relative to the reference polypeptide of SEQ ID NO: 20 and defined as follows:
“+” 1.00 to 1.10,
“++” >1.10,
“+++” >2.10

### Example 3

### Gene Acquisition and Expression of Guanylate Kinase Variants

Synthetic genes encoding N-terminal 6-histidine tagged versions of an evolved guanylate kinase enzyme (GuK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the guanylate kinase expression construct were grown at shake-flask scale, as described in Example 7, (Method 1). Cells were then lysed, purified, and dialyzed into storage buffer (20 mM Tris-HCl, pH 7.4, 100 mM KCl, 0.1 mM EDTA, and 50% glycerol), as described in Example 7. After overnight dialysis, protein samples were removed, and guanylate kinase enzyme concentrations were measured by absorption at 280 nm using a NanoDrop™ 1000 spectrophotometer. Soluble protein concentrations are summarized in Table 3.1 below.

TABLE 3.1
Soluble Enzyme Production of guanylate kinase variant

| SEQ ID NO: | | Soluble Enzyme Concentration |
|---|---|---|
| (nt/aa) | Source organism of GuK gene sequence | After Purification [mg/mL] |
| 17/18 | Branchiostoma floridae | + |

Levels of increased soluble enzyme production are defined as follows:
“+” >12

### Example 4

### Gene Acquisition and Expression of Pyruvate Oxidase

Synthetic genes encoding N-terminal 6-histidine tagged versions of wild-type (WT) pyruvate oxidase (POX) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the pyruvate oxidase expression construct were grown at shake-flask scale and the expressed enzymes were collected as lyophilized powders as described in Example 7, (Method 3). The relative expression levels of these enzymes were determined by gel electrophoresis. The relative expression levels as measured by gel electrophoresis are shown in Table 4.1 below.

Cells transformed with the pyruvate oxidase expression construct were grown at shake-flask scale, as described in Example 7, (Method 1). Cells were then lysed, purified, and dialyzed into storage buffer (20 mM Tris-HCl, pH 7.4, 100 mM KCl, 0.1 mM EDTA, and 50% glycerol), as described in Example 7. After overnight dialysis, protein samples were removed, and pyruvate kinase enzyme concentrations were measured by absorption at 280 nm using a NanoDrop™ 1000 spectrophotometer. Soluble protein concentrations are summarized in Table 4.1 below.

TABLE 4.1
Enzyme Production of pyruvate oxidase variants

| SEQ ID NO: | Source organism of pyruvate oxidase gene | Relative Enzyme expression |
|---|---|---|
| (nt/aa) | sequence | level |
| 31/32 | Bifidobacterium mongoliense | +++ |
| 33/34 | Alkalibacterium subtropicum | ++ |
| 35/36 | Pisciglobus halotolerans | ++ |
| 37/38 | Jeotgalibaca sp PTS2502 | ++ |
| 39/40 | Vagococcus fluvialis | ++ |
| 41/42 | Candidatus Gracilibacteria bacterium | ++ |
| 43/44 | Bavariicoccus seileri | ++ |
| 45/46 | Bifidobacterium aquikefiri | + |
| 47/48 | Aerococcus urinae | + |
| 29/30 | Aerococcus suis | + |

Levels of increased enzyme production were qualitatively determined and defined as follows:
“+” low expression,
“++” moderate expression,
“+++” high expression

### Example 5

### Gene Acquisition and Expression of Adenosine Kinase Variants

Synthetic genes encoding N-terminal 6-histidine tagged versions of three wild-type (WT) adenosine kinase enzymes (AdoK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the adenosine kinase (AdoK) expression constructs were grown at shake-flask scale using IPTG induction, as described in Example 7 (Method 1). The cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and adenosine kinase enzyme concentrations were measured by absorption at 280 nm using a NanoDrop™ 1000 spectrophotometer. Soluble protein concentrations are summarized in Table 5.1 below.

TABLE 5.1
Soluble Enzyme Production of Adenosine Kinase Variants

| SEQ ID NO: | Source organism of adenosine kinase gene | FIOP Soluble Enzyme Production |
|---|---|---|
| (nt/aa) | sequence | (Relative to SEQ ID NO: 50) |
| 51/52 | Thermostaphylospora chromogena | ++ |
| 53/54 | Carbonactinospora thermoautotrophica | ++ |
| 49/50 | Xanthomonas campestris | + |

“Levels of increased soluble enzyme production were determined relative to the reference polypeptide of SEQ ID NO: 50 and defined as follows:
““+”” 1.00 to 1.19,
“++” >1.19

### Example 6

### 3OK Expression and Purification in High Throughput (HTP)

### High Throughput (HTP) Growth of 3OK Enzyme and Variants

Transformed E. coli cells were selected by plating onto LB agar plates containing 1% glucose and 30 μg/ml chloramphenicol. After overnight incubation at 37° C., colonies were placed into the wells of 96-well shallow flat bottom NUNC™ (Thermo-Scientific) plates filled with 180 μl/well LB medium supplemented with 1% glucose and 30 μg/ml chloramphenicol. The cultures were allowed to grow overnight for 18-20 hours in a shaker (200 rpm, 30° C., and 85% relative humidity; Kuhner). Overnight growth samples (20 μL) were transferred into Costar 96-well deep plates filled with 380 μL of Terrific Broth supplemented with 30 μg/ml chloramphenicol. The plates were incubated for 120 minutes in a shaker (250 rpm, 30° C., and 85% relative humidity; Kuhner) until the OD₆₀₀ reached between 0.4-0.8. The cells were then induced with 40 μL of 10 mM IPTG in sterile water and incubated overnight for 18-20 hours in a shaker (250 rpm, 30° C., and 85% relative humidity; Kuhner). The cells were pelleted (4,000 rpm for 20 min), the supernatants were discarded, and the cells were frozen at −80° C. prior to analysis.

For lysis, 200 μL lysis buffer containing 50 mM Tris-HCl buffer, pH 7.5, and 0.1 g/L lysozyme were added to the cell pellet in each well. The cells were shaken vigorously at room temperature for 10 minutes on a bench top shaker. A 100-uL aliquot of the re-suspended cells was transferred to a 96-well format 200 μL BioRad PCR plate, then briefly spun-down prior to 1-hour heat treatment at the temperature indicated, typically 48-60° C. Following heat-treatment, the cell debris was pelleted by centrifugation (4,000 rpm, 4° C., 20 min), and clear supernatants were then used in biocatalytic reactions to determine their activity levels.

### Example 7

### Shake Flask Expression and Purification Procedure

### Shake Flask Expression

### Method 1: Shake Flask Expression Using IPTG Induction

Selected HTP cultures, grown as described in Example 6, were plated onto LB agar plates with 1% glucose and 30 μg/mL chloramphenicol and grown overnight at 37° C. A single colony from each culture was transferred to 5 mL of LB broth with 1% glucose and 30 μg/mL chloramphenicol. The cultures were grown for 20 h at 30° C., 250 rpm, and subcultured at a dilution of approximately 1:50 into 250 mL of Terrific Broth with 30 μg/mL of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 μm PES membrane to further clarify the lysate.

### Method 2: Shake Flask Expression Using Auto-Induction

Selected HTP cultures, grown as described in Example 6, were plated onto LB agar plates with 1% glucose and 30 μg/mL chloramphenicol and grown overnight at 37° C. A single colony from each culture was transferred to 160 mL of Terrific Broth containing 0.075% glucose, 0.03% lactose, and 30 μg/mL of chloramphenicol. The cultures were grown for 20 h at 30° C. and 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 μm PES membrane to further clarify the lysate.

### Method 3: Shake-Flask Expression Using IPTG Induction and Collection of Enzyme as Shake Flask Powder

Selected HTP cultures, grown as described in Example 6, were plated onto LB agar plates with 1% glucose and 30 μg/mL chloramphenicol and grown overnight at 37° C. A single colony from each culture was transferred to 5 mL of LB broth with 1% glucose and 30 μg/mL chloramphenicol. The cultures were grown for 20 h at 30° C., 250 rpm, and subcultured at a dilution of approximately 1:50 into 250 mL of Terrific Broth with 30 μg/mL of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.). The supernatant was collected in petri dishes and frozen at −80° C. The water was then removed under reduced pressure with a lyophilizer. The resultant powder was then collected and stored at −20° C.

Purification of from Shake Flask Lysates

Lysates were supplemented with 1/10^{th} volume of SF elution buffer (50 mM Tris-HCl, 500 mM NaCl, 250 mM imidazole, 0.02% v/v Triton X-100 reagent). Lysates were then purified using an AKTA Pure purification system and a 5 mL HisTrap FF column (GE Healthcare) using the run parameters in Table 7.1. The SF wash buffer comprised 50 mM Tris-HCl, 300 mM NaCl, 20 mM imidazole, 0.02% v/v Triton X-100 reagent.

TABLE 7.1
Purification Parameters

| Parameter | Volume | |
|---|---|---|
| Column volume | 5 | mL |
| Flow rate | 8 | mL/min |
| Pressure limit | 0.3 | mPa |
| Sample volume | 35 | mL |
| Equilibration volume | 5 column volumes (CV) = 25 mL | |
| Wash Unbound volume | 20 CV = 100 mL | |
| Elution | Isocratic (step) | |
| Elution volume | 5 CV = 25 mL | |
| Fraction volume | 1.5 | mL |

| | |
|---|---|
| RE-equilibration volume | 5 CV = 25 mL |

Elution fractions containing protein were identified by UV absorption (A280) and pooled, then dialyzed overnight in dialysis buffer (20 mM Tris-HCl, pH 7.4, 100 mM KCl, 0.1 mM EDTA, and 50% glycerol) in a 3.5K Slide-A-Lyzer™ dialysis cassette (Thermo Fisher) for buffer exchange. Protein concentrations in the preparations were measured by absorption at 280 nm, and preparations were stored at −20° C.

### Example 8

### High Performance Liquid Chromatography (HPLC) Analysis of Nucleotides

### Sample Preparation for Reaction Analysis Using HPLC:

Reactions were quenched by the addition of 4 volume equivalents (5× dilution) or 34 volume equivalents (35× dilution) of 75% v/v MeOH/water. The plate was sealed, mixed well, and centrifuged at 4,000 rpm for 4 min at 4° C. The supernatant was collected and analyzed by HPLC using an Ultimate 3000 system.

TABLE 8.1
Method 1: Achiral HPLC Parameters (Acetonitrile Gradient)

| Instrument | Ultimate 3000 HPLC System with a PAL autosampler | |
|---|---|---|
| Column | Agilent Zorbax RR StableBond Aq, 150 × 3.0 mm × | |
| | 3.5 μm | |
| Guard Column | Agilent Zorbax StabeBond Aq, 5.0 × 3.0 mm, 1.8 μm | |
| Mobile Phases | A: 50 mM sodium phosphate, 2 mM | |
| | tetrabutylammonium bisulfate, pH 7.0 | |
| | B: Acetonitrile | |
| LC Gradient | 0-3.0 | min: 0-20% B |
| | 3.0-3.5 | min: 20% B |
| | 3.5-4.0 | min: 20-0% B |
| | 4.0-6.5 | min: 0% B |
| Flow rate | 1.5 | mL/min |
| Run time | 6.5 | min |
| Column | 30° | C. |
| temperature | | |
| Injection volume | 10 | μL |
| UV Detector | 254 | nm |

TABLE 8.2
Method 2: Achiral HPLC Parameters (Ion Pairing Gradient)

| Instrument | Ultimate 3000 HPLC System with a PAL autosampler | |
|---|---|---|
| Column | Agilent Zorbax RR StableBond Aq, 150 × 3.0 mm × | |
| | 3.5 μm | |
| Guard Column | Agilent Zorbax StabeBond Aq, 5.0 × 3.0 mm, 1.8 μm | |
| Mobile Phases | A: 50 mM sodium phosphate, 2 mM | |
| | tetrabutylammonium bisulfate, pH 7.0 | |
| | B: Acetonitrile | |
| | C: Water | |
| LC Gradient | 0-4.0 | min: 9.0% B, 48.5-16.2% C |
| | 4.0-4.5 | min: 9.0% B, 16.2-48.5% C |
| | 4.5-5.5 | min: 9.0% B, 48.5% C |
| Flow rate | 1.2 | mL/min |
| Run time | 5.5 | min |
| Column | 30° | C. |
| temperature | | |
| Injection volume | 10 | μL |
| UV Detector | 254 | nm |

TABLE 8.3
Method 3: Achiral HPLC Parameters (Isocratic)

| Instrument | Ultimate 3000 HPLC System with a PAL autosampler | |
|---|---|---|
| Column | Agilent Zorbax RR StableBond Aq, 150 × 3.0 mm × | |
| | 3.5 μm | |
| Guard Column | Agilent Zorbax StabeBond Aq, 5.0 × 3.0 mm, 1.8 μm | |
| Mobile Phases | A: 50 mM sodium phosphate, 2 mM | |
| | tetrabutylammonium bisulfate, pH 7.0 | |
| | B: Acetonitrile | |
| LC Gradient | 0-4.0 | min: 7.0% B |
| Flow rate | 1.2 | mL/min |
| Run time | 4.0 | min |
| Column | 30° | C. |
| temperature | | |
| Injection volume | 10 | μL |
| UV Detector | 254 | nm |

### Example 9

### Acetate Kinase (ACK) Expression and Purification

A previously engineered acetate kinase enzyme (ACK-101) featuring an N-terminal 6-histidine tag (See e.g., PCT/US22/23039, which is hereby incorporated by reference in its entirety) was produced in shake flask using IPTG induction according to Example 7, Method 1.

### Example 10

### Shake-Flask Screening of WT 3OK Homologs for Activity on ATP

Eight 3OK WT homologs were produced in shake flask and purified, as described in Example 7. ACK-101 was produced, as described in Example 9.

The 3OK homologs were screened for conversion of ATP to AQP, as depicted above in Scheme 1. Reactions were performed at 100 μL scale in Costar 96-well deep plates. Reactions included 1 mM ATP, 10 mM LiKAcPO₄, 1 mM MgCl₂, 0.2 g/L ACK-101, 0.5 g/L 30K, in 50 mM Tris-HCl (pH 7.5). The reactions were set up by sequential addition of 5× stocks prepared in 50 mM Tris-HCl (pH 7.5) as follows: (i) 20 μL of a 1.0 g/L ACK-101 stock was added; (ii) 20 μL of a 50 mM LiKAcPO₄ stock was added; (iii) 20 μL of a 2.5 g/L stock of 3OK purified enzyme variant was added; (iv) 20 μL of a 5 mM MgCl₂ was added; (v) 20 μL of a 5 mM ATP stock was added. After mixing well and briefly centrifuging to collect the contents in the bottom of each well, the reaction plates were heat-sealed with a peelable aluminum seal and incubated for 18 h (400 rpm, 30° C.).

Reactions were analyzed by HPLC Method 1, as described in Example 8, and the results for reaction with ATP to produce AQP are shown in their respective columns in Table 10.1.

TABLE 10.1
Conversion of 3OKs with Native ATP

| SEQ ID NO: | | |
|---|---|---|
| (nt/aa) | Source organism of 3OK gene sequence | Conversion of ATP to AQP |
| 3/4 | E coli W3110 | ++ |
| 5/6 | E coli W3110 | ++ |
| 7/8 | Thermomonas hydrothermalis | |
| 9/10 | Geobacillus stearothermophilus | ++ |
| 11/12 | Aquifex aeolicus | + |
| 13/14 | Thermotoga sp. RQ7 | +++ |
| 15/16 | Caldibacillus thermoamylovorans | +++ |

Levels of conversion of ATP to 3′-phosphorylated ATP (AQP) are defined as follows:
“+” 0.6-4.5 percent conversion,
“++” >4.5 percent conversion,
“+++” >41 percent conversion

### Example 11

Enzymatic Synthesis of NQP from Nucleoside

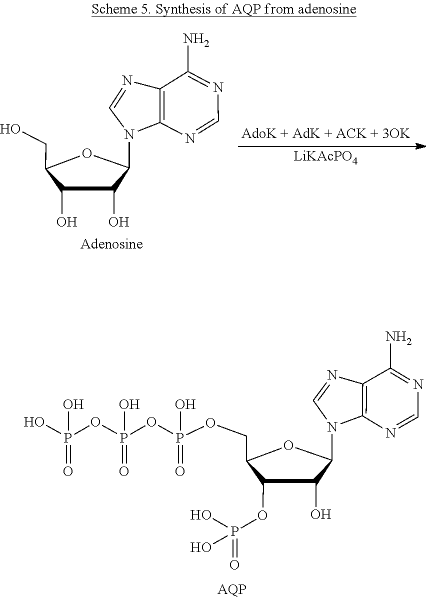

ACK-101 was produced and purified, as described in Example 9. AdoK (SEQ ID NO: 50) was produced and purified, as described in Example 3. AdK (SEQ ID NO: 26) was produced and purified, as described in Example 2. 3OK enzyme variants (SEQ ID NO: 14) were produced as described in Example 7 and tested in a kinase cascade resulting in the conversion of substrate nucleoside to the respective NQP.

Reactions were performed in 200 μL BioRad PCR plates. As depicted in Scheme 5 (and more generally in Scheme 3, above), reactions included 1.11 mM nucleoside, 11.1 mM LiKAcPO₄, 11.1 mM MgSO₄, 1.3 g/L ACK-101, 1.1 mg/mL 3OK (SEQ ID NO: 10 or SEQ ID NO: 14), 0.5 g/L AdK (SEQ ID NO: 26), 2.8 g/L AdoK (SEQ ID NO: 50), and 11.1 mM Tris-HCl (pH 8).

All reagents were dissolved in water. The reactions were prepared as follows: to a well was added sequentially by micropipette 10 μL 100 mM MgSO₄, 10 μL 100 mM Tris-HCl (pH 8), 10 μL 0.1 mM ATP, 10 μL 10 mM nucleoside 10 μL 11.5 mg/mL ACK-101, 10 μL 4.8 mg/mL AdK (SEQ ID NO: 26), 10 μL 25 mg/mL AdoK (SEQ ID NO: 50), 10 μL 10 mg/mL 3OK enzyme variant (SEQ ID NO: 10 or SEQ ID NO: 14), and then 10 μL 100 mM LiKAcPO₄. The plate was sealed and shaken at 400 rpm in an incubator set at 30° C. for 24 hours.

Subsequently, the reactions were quenched by removing 30 μL of the reaction mixtures, and four volume equivalents of 75% MeOH/Water (120 μL, 5× dilution) were added, as described in Example 8. Samples were analyzed by HPLC Method 2—ion pairing gradient and the results are shown in Table 11.1.

TABLE 11.1
Reaction of 3OK enzyme variants with ATP

| SEQ ID NO: | | |
|---|---|---|
| (nt/aa) | NTP Substrate | Percent Conversion to NQP |
| 13/14 | ATP | + |

Level of conversion of ATP substrate to AQP are defined as follows:
“+” >1.5 percent conversion

### Example 12

POX-Driven Enzymatic Synthesis of ATP from Adenosine without Addition of Acetyl Phosphate

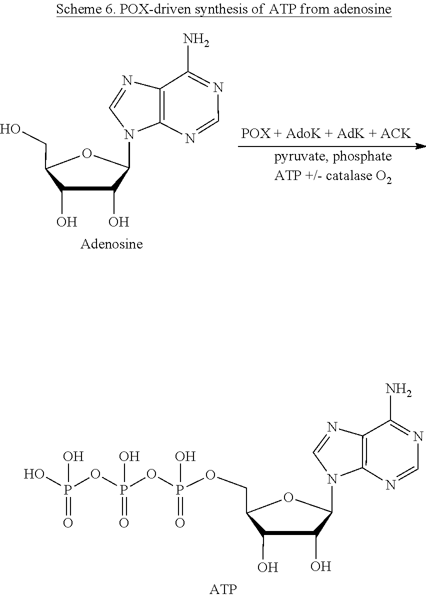

ACK-101 was produced and purified, as described in Example 9. AdoK (SEQ ID NO: 50) was produced and purified, as described in Example 3. AdK (SEQ ID NO: 26) was produced and purified, as described in Example 2. Pyruvate Oxidase (POX) (SEQ ID NO: 40) was produced and purified, as described in Example 4 and tested in a kinase cascade. Use of POX precludes the need for added LiKAcPO₄ in the kinase cascade.

Reactions were performed in 1.1 mL Axygen deepwell plates. As depicted in Scheme 6 (and more generally in Scheme 4, above), reactions included 0.91 mM adenosine, 0.009 mM ATP, 9.1 mM MgSO₄, 1 g/L ACK-101, 0.4 g/L AdK (SEQ ID NO: 26), 0.46 g/L AdoK (SEQ ID NO: 50), 9.1 mM Tris-HCl (pH 8), 0.45 mM flavin adenine dinucleotide (FAD), 0.45 mM thiamine pyrophosphate (ThPP), 45.5 mM sodium pyruvate, 18 mM K₂HPO₄, and 0.26 g/L POX (SEQ ID NO: 40).

All reagents were dissolved in water. The reactions were prepared as follows: to a well was added sequentially by micropipette 10 μL 100 mM MgSO₄, 10 μL 100 mM Tris-HCl (pH 8), 10 μL 200 mM K₂HPO₄, 10 μL 10 mM adenosine 5 μL 10 mM FAD, 5 μL 10 mM ThPP, 10 μL 0.1 mM ATP, 10 μL 11.5 mg/mL ACK-101, 10 μL 4.8 mg/mL AdK (SEQ ID NO: 26), 10 μL 5.04 mg/mL AdoK (SEQ ID NO: 50), 10 μL 0.26 mg/mL POX (SEQ ID NO: 40), and 10 μL 500 mM pyruvate. The plate was sealed with a porous aeroseal and shaken at 300 rpm in an incubator set at 30° C. and 85% humidity for 17 hours.

Subsequently, the reactions were quenched by transferring 60 μL of the reaction mixture into 60 μL methanol. The quenched mixture was filtered with a 0.45 μm low-binding hydrophilic PTFE plate. A 20-μL aliquot of the filtrate was then diluted with 180 μL water. These samples were then analyzed by HPLC Method 2—ion pairing gradient in Example 8, and the results are shown in Table 12.1.

TABLE 12.1
POX-driven synthesis of ATP from adenosine

| POX SEQ ID NO: (nt/aa) | Percent conversion to ATP |
|---|---|
| 39/40 | + |

Levels of conversion of adenosine to ATP are defined as follows:
“+” >75 percent conversion

### Example 13

### IMPROVEMENTS Over SEQ ID NO: 10 in Conversion to Tetraphosphorylated Nucleotide

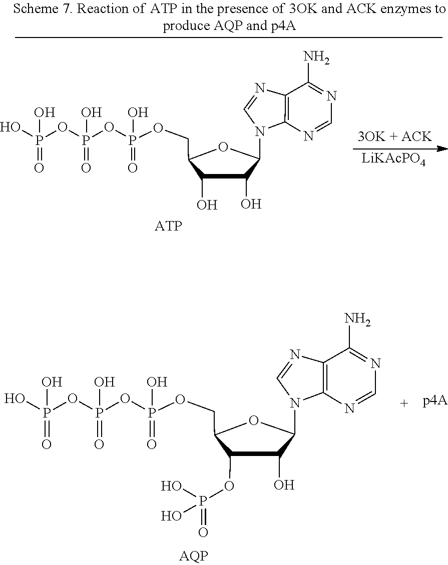

Libraries of engineered genes were produced from the parent gene SEQ ID NO: 10 using various techniques (e.g., saturation mutagenesis and recombination of previously identified beneficial mutations). The polypeptides encoded by each gene were produced in HTP and prepared, as described in Example 6.

Reactions were performed at 2 μL scale in 96-well format 200 μL BioRad PCR plates. Reactions included 10 mM ATP, 50 mM LiKAcPO₄, 10 mM MgCl₂, 50% v/v lysate, in 50 mM Tris-HCl (pH 7.5). The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for 18 hours.

Reactions were quenched according to Example 8 by adding 34 volume equivalents (65 μL, 35× dilution) of a 75% v/v MeOH/water solution to each well. After mixing well and centrifuging, the samples were subjected to HPLC analysis according to the ion pairing gradient HPLC Method 2.

### Improved Conversion for AQP

Activity relative to SEQ ID NO: 10 was calculated as AQP product peak area of the variant compared with the product peak area observed by the reaction with SEQ ID NO: 10. The results are shown below in Table 13.1.

TABLE 13.1
Improved Conversion to AQP of Variants
Relative to SEQ ID NO: 10

| | Amino Acid Differences | |
|---|---|---|
| SEQ ID NO: | (Relative to SEQ ID | FIOP Conversion to AQP |
| (nt/aa) | NO: 10) | (Relative to SEQ ID NO: 10) |
| 55/56 | M165S | +++ |
| 57/58 | S13V | ++ |
| 59/60 | R93Y | ++ |
| 61/62 | V74K | ++ |
| 63/64 | A38E | + |
| 65/66 | V39L | + |
| 67/68 | S13A | + |
| 69/70 | H89L | + |
| 71/72 | T124W | + |
| 73/74 | A72R | + |
| 75/76 | T124V | + |

Levels of increased conversion to AQP were determined relative to the reference polypeptide of SEQ ID NO: 10 and defined as follows:
“+” 1.20 to 1.30,
“++” >1.30,
“+++” >1.7

FIOP for Phosphorylated Product (p4A) The structure of the primary byproduct has been preliminarily assigned as that of adenosine-5′-tetraphosphate (ppppA or p4A). Other phosphorylated products are also potentially formed at low levels. The structures of the potential byproducts are shown, but not limited to, those shown above in Scheme 2.

In applications where p4A is the desired product, enzyme variants with improved p4A activity will be of interest. Activity relative to SEQ ID NO: 10 was calculated as the p4A product peak area of the variant compared with the p4A product peak area observed by the reaction with SEQ ID NO: 10. The results are shown below in Table 13.2.

TABLE 13.2
Improved Conversion to p4A of Variants
Relative to SEQ ID NO: 10

| | Amino Acid Differences | |
|---|---|---|
| SEQ ID NO: | (Relative to SEQ ID | FIOP Conversion to p4A |
| (nt/aa) | NO: 10) | (Relative to SEQ ID NO: 10) |
| 77/78 | N178H | +++ |
| 79/80 | D138-/V139- | +++ |
| 81/82 | T17R | +++ |
| 83/84 | N148R | ++ |
| 85/86 | L123A | ++ |
| 87/88 | D177S | ++ |
| 89/90 | L123I | ++ |
| 91/92 | R41M | ++ |
| 93/94 | N179M | + |
| 95/96 | N178R | + |
| 97/98 | F150M | + |
| 99/100 | R60L | + |
| 101/102 | N148G | + |
| 55/56 | M165S | + |
| 103/104 | W163S | + |
| 105/106 | L144V | + |
| 107/108 | D138S | + |

Levels of increased conversion to p4A were determined relative to the reference polypeptide of SEQ ID NO: 10 and defined as follows:
“+” 1.40 to 2.00,
“++” >2.00,
“+++” >4.50

The change in AQP selectivity relative to SEQ ID NO: 10 was calculated as the percent selectivity for AQP divided by the percent selectivity for AQP in the reaction with SEQ ID NO: 10.

TABLE 13.3
Improved Selectivity for AQP of Variants
Relative to SEQ ID NO: 10

| | Amino Acid Differences | |
|---|---|---|
| SEQ ID NO: | (Relative to SEQ ID | FIOP Selectivity for AQP |
| (nt/aa) | NO: 10) | (Relative to SEQ ID NO: 10) |
| 69/70 | H89L | +++ |
| 109/110 | H89S | +++ |
| 111/112 | V92A | ++ |
| 61/62 | V74K | ++ |
| 113/114 | A36T | ++ |
| 115/116 | H89G | ++ |
| 117/118 | V74M | ++ |
| 65/66 | V39L | + |
| 119/120 | A35S | + |
| 57/58 | S13V | + |
| 121/122 | A32G | + |
| 123/124 | F150W | + |
| 125/126 | N76A | + |
| 127/128 | V40M | + |
| 129/130 | L156M | + |

Levels of increased AQP selectivity were determined relative to the reference polypeptide of SEQ ID NO: 10 and defined as follows:
““+”” 1.10 to 1.18,
““++”” >1.18,
““+++”” >1.21

The change in p4A selectivity relative to SEQ ID NO: 10 was calculated as the percent selectivity for p4A divided by the percent selectivity for p4A in the reaction with SEQ ID NO: 10.

TABLE 13.4
Improved Selectivity for p4A of Variants
Relative to SEQ ID NO: 10

| | Amino Acid Differences | |
|---|---|---|
| SEQ ID NO: | (Relative to SEQ ID | FIOP Selectivity for p4A |
| (nt/aa) | NO: 10) | (Relative to SEQ ID NO: 10) |
| 95/96 | N178R | +++ |
| 77/78 | N178H | +++ |
| 83/84 | N148R | +++ |
| 79/80 | D138-/V139- | ++ |
| 81/82 | T17R | ++ |
| 105/106 | L144V | ++ |
| 131/132 | L123V | ++ |
| 87/88 | D177S | ++ |
| 97/98 | F150M | + |
| 89/90 | L123I | + |
| 133/134 | T17Q | + |
| 101/102 | N148G | + |
| 135/136 | N178S | + |
| 85/86 | L123A | + |
| 91/92 | R41M | + |
| 137/138 | P116S | + |
| 93/94 | N179M | + |

Levels of increased p4A selectivity were determined relative to the reference polypeptide of SEQ ID NO: 10 and defined as follows:
““+”” 1.20 to 2.70,
““++”” >2.70,
““+++”” >4.60

Beneficial selectivity and activity mutations favoring AQP relative to SEQ ID NO: 10 were recombined and were produced in HTP and prepared, as described in Example 6.

Reactions were performed at 2 μL scale in 96-well format 200 μL BioRad PCR plates. Reactions included 10 mM ATP, 50 mM LiKAcPO₄, 10 mM MgCl₂, 25% v/v lysate, in 50 mM Tris-HCl (pH 7.5). The reactions were set up as follows: (i) all reaction components except for lysate were pre-mixed in a single solution, and 1 μL of this solution was aliquoted into each well of the 96-well plate; (ii) 0.5 50 mM Tris-HCl (pH 7.5) was added to each well; (iii) 0.5 μL lysate was added to each well. After briefly centrifuging to collect the contents in the bottom of each well, the reaction plates were heat-sealed with a peelable aluminum seal and incubated at 30° C. for 18 hours.

Reactions were quenched according to Example 8 by adding 34 volume equivalents (65 μL, 35× dilution) of a 75% v/v MeOH/water solution to each well. After mixing well and centrifuging, the samples were subjected to HPLC analysis according to the ion pairing gradient HPLC Method 2.

Activity relative to SEQ ID NO: 10 was calculated as AQP product peak area of the variant compared with the product peak area observed by the reaction with SEQ ID NO: 10. The results are shown below in Table 13.5.

TABLE 13.5
Improved Conversion to and Selectivity for
AQP of Variants Relative to SEQ ID NO: 10

| | | | FIOP Selectivity |
|---|---|---|---|
| | Amino Acid Differences | FIOP Conversion to | for AQP (Relative |
| SEQ ID NO: | (Relative to | AQP (Relative to | to SEQ ID NO: |
| (nt/aa) | SEQ ID NO: 10) | SEQ ID NO: 10)¹ | 10)² |
| 139/140 | S13V/A36T/A38E/V40M/A72R/ | +++ | ++ |
| | V74K/H89L/R93Y | | |
| 141/142 | N76A/H89G/R93Y/M165S | +++ | +++ |
| 143/144 | A36T/V40M/A72R/V74K/H89G/ | +++ | ++ |
| | R93Y | | |
| 145/146 | A72R/H89L/M165S | +++ | ++ |
| 147/148 | S13V/A38E/V39L/V40M/H89L/ | +++ | +++ |
| | T124W/L156M | | |
| 149/150 | A36T/A38E/V40M/A72R/V74K/ | +++ | ++ |
| | H89S/R93Y/T124W/L156M | | |
| 151/152 | S13V/V74K/H89L/R93Y | +++ | ++ |
| 153/154 | N76A/H89L/T124V/M165S | +++ | ++ |
| 155/156 | S13A/A38E/V40M/H89G | +++ | ++ |
| 157/158 | S13A/A36T/A38E/V40M/A72R/ | +++ | ++ |
| | N76A/H89G | | |
| 159/160 | S13V/A38E/V39L/V40M/A72R/ | +++ | ++ |
| | N76A/R93Y/M165S | | |
| 161/162 | V74K/H89L/R93Y/L156M | +++ | ++ |
| 163/164 | S13A/A36T/V40M/A72R/V74K/ | +++ | ++ |
| | N76A/R93Y | | |
| 165/166 | N76A/H89G/M165S | +++ | ++ |
| 167/168 | S13V/A36T/A38E/V40M/A72R/ | +++ | +++ |
| | V74K/R93Y/L156M | | |
| 169/170 | S13V/A72R/N76A/H89G/R93Y | +++ | ++ |
| 171/172 | S13V/A72R/H89L/T124W | +++ | ++ |
| 173/174 | S13A/A72R/H89L/T124W | +++ | +++ |
| 175/176 | S13V/A72R/V74K/N76A/H89L/ | +++ | ++ |
| | R93Y | | |
| 177/178 | S13A/V40M/A72R/N76A/H89L | +++ | +++ |
| 179/180 | A36T/A38E/V39L/V40M/V74K/ | +++ | ++ |
| | N76A/H89L/R93Y/T124V | | |
| 181/182 | S13V/A38E/V40M/H89G | +++ | ++ |
| 183/184 | S13V/A72S/V74K/H89L/R93Y | +++ | ++ |
| 185/186 | S13V/A36T/A38E/V40M/H89G | ++ | ++ |
| 187/188 | S13A/A72R/H89S/R93Y/T124V/ | ++ | ++ |
| | L156M | | |
| 189/190 | S13A/H89L/M165S | ++ | ++ |
| 191/192 | S13V/A72R/N76A/H89L/T124V | ++ | ++ |
| 193/194 | H89L/R93Y/T124W | ++ | ++ |
| 195/196 | V74K/N76A/H89L/R93Y/T124V | ++ | ++ |
| 197/198 | S13A/A36T/A38E/V40M/N76A/ | ++ | ++ |
| | H89S/R93Y | | |
| 199/200 | S13A/A36T/V40M/R93Y/T124V | ++ | ++ |
| 201/202 | S13V/A72R/R93Y/T124W | ++ | +++ |
| 203/204 | A38E/V40M/N76A/H89L/T124W | ++ | ++ |
| 205/206 | A72R/V74K/H89L/R93Y/T124V | ++ | + |
| 207/208 | S13V/A72R/V74K/H89L/R93Y | ++ | ++ |
| 209/210 | S13V/N76A/H89L/R93Y/L156M/ | ++ | ++ |
| | M165S | | |
| 211/212 | A38E/A72R/H89L/R93Y/T124V | ++ | + |
| 213/214 | S13V/N76A/H89G/R93Y/T124W | ++ | + |
| 215/216 | S13V/V40M | ++ | + |
| 217/218 | A72R/V74K/H89G/R93Y | ++ | + |
| 219/220 | S13V/A36T/A38E/V39L/V40M/ | ++ | +++ |
| | H89L | | |
| 221/222 | V39L/V40M/A72R/N76A | ++ | +++ |
| 223/224 | V74K/H89L/T124W | ++ | +++ |
| 225/226 | A36T/A38E/V39L/V40M/A72R/ | ++ | +++ |
| | V74K/H89L | | |
| 227/228 | S13V/A36T/A38E/V40M/V74K/ | ++ | + |
| | N76A | | |
| 229/230 | A38E/V40M/R93Y | ++ | + |
| 231/232 | S13V/H89L | ++ | + |
| 233/234 | S13A/H89L/T124V | ++ | ++ |
| 235/236 | S13V/A72R/N76A | ++ | + |
| 237/238 | S13V/N76A/H89S/R93Y | ++ | + |
| 239/240 | A36T/V39L/V40M/A72R/N76A/ | ++ | ++ |
| | H89L/R93Y | | |
| 241/242 | S13V/N76A/R93Y | ++ | + |
| 243/244 | S13V/N76A/R93Y/T124V | ++ | + |
| 245/246 | A36T/A38E/V39L/V40M/A72R/ | ++ | + |
| | V74K/N76A/T124W | | |
| 247/248 | S13V/A36T/V39L/V40M/A72R/ | ++ | + |
| | N76A/H89L/T124V | | |
| 249/250 | S13V/N76A/H89L/T124W/L156M | ++ | +++ |
| 251/252 | S13A/A72R/H89G | ++ | +++ |
| 253/254 | S13V/V74K/H89L/R93Y/T124V | + | +++ |
| 255/256 | V74K/H89S/R93Y | + | + |
| 257/258 | A72R/R93Y/T124V | + | + |
| 259/260 | S13V/A36T/A38E/V39L/A72R/ | + | ++ |
| | N76A/H89L/R93Y/T124W | | |
| 261/262 | V74K/N76A/H89L | + | +++ |
| 263/264 | A72R/N76A/H89G/R93Y | + | + |
| 265/266 | A38E/V40M/H89S/T124W | + | + |
| 267/268 | H89L/R93Y | + | +++ |
| 269/270 | S13A/T124V | + | + |
| 271/272 | H89L/T124W | + | + |
| 273/274 | N76A/H89G/R93Y/T124V | + | + |
| 275/276 | S13V/A72R/H89G | + | + |
| 277/278 | A72R/V74K/R93Y | + | +++ |
| 279/280 | S13A/A72R/N76A | + | + |
| 281/282 | A38E/V39L | + | + |
| 283/284 | A36T/V39L/V40M/N76A/R93Y/ | + | + |
| | L156M | | |
| 285/286 | S13V/T124V | + | + |
| 287/288 | S13V/A72R/V74K/N76A/H89L/ | + | + |
| | R93Y/T124V | | |
| 289/290 | S13V/A72R/T124W | + | + |
| 291/292 | S13V/A36T/V40M/A72R/L156M | + | + |
| 293/294 | A72R/V74K/N76A/H89G/T124V | + | + |
| 295/296 | S13A/A36T/A38E/V40M | + | + |
| 297/298 | S13V/A72R | + | + |
| 299/300 | N76A/R93Y | + | + |
| 301/302 | S13A/V74K/H89G/L156M | + | ++ |
| 303/304 | S13V/L156M | + | + |
| 305/306 | S13V/A72R/N76A/T124V/L156M | + | +++ |
| 307/308 | A38V/A72R/N76A/H89G | + | +++ |
| 309/310 | A72R/N76A/T124V | + | + |
| 311/312 | A38E/V40M/A72R | + | + |
| 313/314 | A72R/N76A/T124W | + | + |
| 315/316 | S13A/N76A | + | + |
| 317/318 | T124W/L156M | + | + |
| 319/320 | S13A/A38E/A72R/L156M | + | + |
| 321/322 | V74K/H89L | + | + |
| 323/324 | A72R/V74K/N76A/T124V | + | + |
| 325/326 | A38E/V40M/L156M | + | + |
| 327/328 | A72R/V74K/R93Y/T124V | + | + |
| 329/330 | A72R/N76A | + | + |
| 331/332 | S13A/H89S/M165S | + | +++ |
| 333/334 | S13V/N76A | + | + |
| 335/336 | S13V/H89G | + | + |
| 337/338 | A38E/V39L/N76A | + | + |
| 339/340 | S13V/T124W | + | + |
| 341/342 | S13V/A36T/A38E/V40M/A72R/ | + | + |
| | V74K | | |
| 343/344 | A72R/V74K/H89G | + | + |
| 345/346 | A38E/V39L/V40M/A72R/V74K/ | + | ++ |
| | N76A/H89S | | |
| 347/348 | S13V/A72R/H89L/T124V/M165S | + | +++ |
| 349/350 | S13A/N76A/T124W | + | + |
| 351/352 | S13V/A72R/V74K/H89L/R93Y/ | + | +++ |
| | T124W | | |
| 353/354 | V74K/N76A/H89G/T124V | + | + |
| 355/356 | S13V/H89S | + | +++ |
| 357/358 | S13V/N76A/H89G/L156M/M165S | + | +++ |
| 359/360 | S13V/V74K/H89S/R93Y | + | + |
| 361/362 | A38E/N76A/L156M | + | + |
| 363/364 | S13V/A36T/A38E/V40M/A72R/ | + | + |
| | V74K/T124V | | |
| 365/366 | S13V/A36T/A38E/V39L/V40M/ | + | + |
| | V74K/H89G | | |

¹Levels of increased conversion of ATP to AQP were determined relative to the reference polypeptide of SEQ ID NO: 10 and defined as follows:
“+” 1.25 to 2.04,
“++” >2.04,
“+++” >2.83
²Levels of increased selectivity for AQP were determined relative to the reference polypeptide of SEQ ID NO: 10 and defined as follows:
“+” 1.17 to 1.55,
“++” >1.55,
“+++” >1.58

### Example 14

### Capillary Electrophoresis (CE) Analysis of Oligonucleotides

### Sample Preparation for Reaction Analysis Using CE

Reaction samples were analyzed via capillary electrophoresis using an ABI 3500xl Genetic Analyzer (ThermoFisher). Reactions (2 μL) were quenched by the addition of 38 μL of 10 mM aqueous EDTA. The quenched reaction mixture was further diluted 80000 times by water. 2 μL of this quenched solution was transferred to a new 96-well MicroAmp Optical PCR plate or 384-well MicroAmp Optical PCR plate containing 18 μL Hi-Di™ Formamide (ThermoFisher) which has an appropriate size standard. The ABI3500xl was configured with POP6 polymer, 50 cm capillaries and a 45° C. oven temperature. Pre-run settings were 18 KV for 180 sec. Injection was 5 KV for 5 sec, and the run settings were 19.5 KV for 640 sec. FAM-labeled oligo substrates and products were identified by their sizes relative to the sizing ladder, with the substrate oligo peak at ˜18 or 20 bp and the products appearing in the region of ˜14-18 bp.

Reaction samples were analyzed via capillary electrophoresis using an ABI 3500xl Genetic Analyzer (ThermoFisher). Reactions (2 μL) were quenched by the addition of 38 μL of 10 mM aqueous EDTA. The quenched reaction mixture was further diluted 80000 times by water. 2 μL of this quenched solution was transferred to a new 96-well MicroAmp Optical PCR plate or 384-well MicroAmp Optical PCR plate containing 18 μL Hi-Di™ Formamide (ThermoFisher) which has an appropriate size standard. The ABI3500xl was configured with POP6 polymer, 50 cm capillaries and a 45° C. oven temperature. Pre-run settings were 18 KV for 180 sec. Injection was 5 KV for 5 sec, and the run settings were 19.5 KV for 640 sec. FAM-labeled oligo substrates and products were identified by their sizes relative to the sizing ladder, with the substrate oligo peak at ˜18 or 20 bp and the products appearing in the region of ˜14-18 bp.

TABLE 14.1
List of substrate and product oligonucleotides

| Structural Description | SEQ ID NO: |
|---|---|
| 5′-6-FAM-T15AT*mG | 2129 |
| 5′-6-FAM-T17mAmUmC | 2130 |
| 5′-6-FAM-T15AT*mGrA-3′P | 2131 |
| 5′-6-FAM-T15AT*mGrG-3′P | 2132 |
| 5′-6-FAM-T15AT*mGrC-3′P | 2133 |
| 5′-6-FAM-T15AT*mGrU-3′P | 2134 |
| 5′-6-FAM-T15AT*mG(2′dF)A-3′P | 2135 |
| 5′-6-FAM-T15AT*mG(2′dF)G-3′P | 2136 |
| 5′-6-FAM-T15AT*mG(2′dF)C-3′P | 2137 |
| 5′-6-FAM-T15AT*mG(2′dF)U-3′P | 2138 |
| 5′-6-FAM-T15AT*mGmA-3′P | 2139 |
| 5′-6-FAM-T15AT*mGmG-3′P | 2140 |
| 5′-6-FAM-T15AT*mGmC-3′P | 2141 |
| 5′-6-FAM-T15AT*mGmU-3′P | 2142 |
| 5′-6-FAM-T15AT*mG*rA-3′P | 2143 |
| 5′-6-FAM-T15AT*mG*rG-3′P | 2144 |
| 5′-6-FAM-T17mAmUmCrA-3′P | 2145 |
| 5′-6-FAM-T17mAmUmCrG-3′P | 2146 |
| 5′-6-FAM-T17mAmUmCrC-3′P | 2147 |
| 5′-6-FAM-T17mAmUmCrU-3′P | 2148 |
| 5′-6-FAM-T17mAmUmC(2′dF)A-3′P | 2149 |
| 5′-6-FAM-T17mAmUmC(2′dF)G-3′P | 2150 |
| 5′-6-FAM-T17mAmUmC(2′dF)C-3′P | 2151 |
| 5′-6-FAM-T17mAmUmC(2′dF)U-3′P | 2152 |
| 5′-6-FAM-T17mAmUmCmA-3′P | 2153 |
| 5′-6-FAM-T17mAmUmCmG-3′P | 2154 |
| 5′-6-FAM-T17mAmUmCmC-3′P | 2155 |
| 5′-6-FAM-T17mAmUmCmU-3′P | 2156 |
| 5′-6-FAM-T17mAmUmC*rA-3′P | 2157 |
| 5′-6-FAM-T17mAmUmC*rG-3′P | 2158 |
| 5′-6-FAM-T17mAmUmCdA-3′P | 2159 |
| 5′-6-FAM-T17mAmUmCdG-3′P | 2160 |
| 5′-6-FAM-T17mAmUmCdC-3′P | 2161 |
| 5′-6-FAM-T17mAmUmCdT-3′P | 2162 |

### Example 15

### 3OK Expression and Heat Lysis in High Throughput (HTP) Using Thermal Lysis

### High Throughput (HTP) Growth of 3OK Enzyme and Variants and Thermal Lysis

Transformed E. coli cells were selected by plating onto LB agar plates containing 1% glucose and 30 μg/ml chloramphenicol. After overnight incubation at 37° C., colonies were placed into the wells of 96-well shallow flat bottom NUNC™ (Thermo-Scientific) plates filled with 180 μl/well LB medium supplemented with 1% glucose and 30 μg/ml chloramphenicol. The cultures were allowed to grow overnight for 18-20 hours in a shaker (200 rpm, 30° C., and 85% relative humidity; Kuhner). Overnight growth samples (20 μL) were transferred into Costar 96-well deep plates filled with 380 μL of Terrific Broth supplemented with 30 μg/ml chloramphenicol. The plates were incubated for 120 minutes in a shaker (250 rpm, 30° C., and 85% relative humidity; Kuhner) until the OD₆₀₀ reached between 0.4-0.8. The cells were then induced with 40 μL of 10 mM IPTG in sterile water and incubated overnight for 18-20 hours in a shaker (250 rpm, 30° C., and 85% relative humidity; Kuhner). The cells were pelleted (4,000 rpm for 20 min), the supernatants were discarded, and the cells were frozen at −80° C. prior to analysis.

For lysis, buffer (as specified in each example) and 0.1 g/L lysozyme were added to the cell pellet in each well. The cells were shaken vigorously at room temperature for 10 minutes on a bench top shaker. A 100-μLor 150-μL aliquot of the re-suspended cells was transferred to a 96-well format 200 μL BioRad PCR plate, then briefly spun-down prior to 1-hour heat treatment at specific temperature. Following heat-treatment, the cell debris was pelleted by centrifugation (4,000 rpm, 4° C., 20 min), and clear supernatants were then used in biocatalytic reactions to determine their activity levels.

### Example 16

### Improvements Over SEQ ID NO: 142 in Conversion to Tetraphosphorylated Nucleotide

### HTP Screening for Improved 3OK Variants

SEQ ID NO: 142 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 16.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched.

Reactions were quenched according to Example 8 by adding 34 volume equivalents (65 μL, 35× dilution) of a 75% v/v MeOH/water solution to each well. After mixing well and centrifuging, the samples were subjected to HPLC analysis according to the ion pairing gradient HPLC Method 2.

TABLE 16.1
All lysis, reaction, quench, and analytical properties

Lysis conditions: Lysis buffer - 50 mM Tris-HCl buffer, pH 7.5, 0.1 g/L lysozyme; Lysis buffer
volume - 200 μL; Lysate pre-treatment - Lysates were heat-treated at 55° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO₄, 10 mM Magnesium chloride, 0.2 g/L AcK101; Lysate dilution - None; Reaction time - 18
hour
Quench conditions: Reactions were quenched and analyzed by HPLC as described in Example 8

Activity relative to SEQ ID NO: 2 (Activity FIOP) was calculated as the fold-improvement of conversion of the variant divided by the conversion observed in the reaction with SEQ ID NO: 2 (where the conversion may be set as the average of replicates or else the highest single sample as appropriate). Conversion was calculated as 3′PO4 product peak areas over the total of the unreacted substrate, byproduct, and 3′PO4 product peak areas. The results are shown in Table 16.2.

TABLE 16.2
Relative to SEQ ID NO: 142

| | Amino Acid Differences | Activity FIOP |
|---|---|---|
| SEQ ID | (Relative to | Conversion Relative |
| NO: (nt/aa) | SEQ ID NO: 142) | to SEQ ID NO: 142 |
| 371/372 | S13E/A76S/Y93Q/A198L | +++ |
| 373/374 | T181N | ++ |
| 375/376 | A68G/H103V/T181N/I182E | + |
| 377/378 | I182E | + |
| 379/380 | Y93L/A198L | + |
| 381/382 | S13E/A76S/Y93Q | + |
| 383/384 | K82T/A198I | + |
| 385/386 | S13E/A76S/A198L | + |

Levels of increased activity were determined relative to the reference polypeptide of SEQ ID NO: 142 and defined as follows:
“+” 1.0 to 1.5,
“++” 1.5 to 2.4,
“+++” 2.4.

### Example 17

### Improvements Over SEQ ID NO: 142 in Conversion to Tetraphosphorylated Nucleotide

### HTP Screening for Improved 3OK Variants

SEQ ID NO: 142 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 17.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched.

Reactions were quenched according to Example 8 by adding 34 volume equivalents (65 μL, 35× dilution) of a 75% v/v MeOH/water solution to each well. After mixing well and centrifuging, the samples were subjected to HPLC analysis according to the ion pairing gradient HPLC Method 2.

TABLE 17.1
All lysis, reaction, quench, and analytical properties

Lysis conditions: Lysis buffer - 50 mM Tris-HCl buffer, pH 7.5, 0.1 g/L lysozyme; Lysis buffer
volume - 200 μL; Lysate pre-treatment - Lysates were heat-treated at 60° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO₄, 10 mM Magnesium chloride, 0.2 g/L AcK101; Lysate dilution - None; Reaction time - 1
hour

Activity relative to SEQ ID NO: 142 (Activity FIOP) was calculated as the fold-improvement of conversion of the variant divided by the conversion observed in the reaction with SEQ ID NO: 142 (where the conversion may be set as the average of replicates or else the highest single sample as appropriate). Conversion was calculated as 3′P04 product peak areas over the total of the unreacted substrate, byproduct, and 3′P04 product peak areas. The results are shown in Table 17.2.

TABLE 17.2
Relative to SEQ ID NO: 142

| SEQ ID | | Activity FIOP |
|---|---|---|
| NO: | Amino Acid Differences | Conversion Relative to |
| (nt/aa) | (Relative to SEQ ID NO: 142) | SEQ ID NO: 142 |
| 387/388 | G200C | +++ |
| 389/390 | Y93L | +++ |
| 391/392 | H103L | +++ |
| 393/394 | Y93M | +++ |
| 395/396 | Y93E | ++ |
| 397/398 | Y93Q | ++ |
| 399/400 | V111I | ++ |
| 387/388 | G200C | +++ |
| 389/390 | Y93L | +++ |
| 391/392 | H103L | +++ |
| 393/394 | Y93M | +++ |
| 395/396 | Y93E | ++ |
| 397/398 | Y93Q | ++ |
| 399/400 | V111I | ++ |
| 401/402 | H210S | ++ |
| 403/404 | A191D | ++ |
| 405/406 | H210E | ++ |
| 407/408 | K82E | ++ |
| 409/410 | Y93C | + |
| 411/412 | Y93V | + |
| 413/414 | A76S | + |
| 415/416 | H210P | + |
| 417/418 | H211K | + |
| 419/420 | G200D | + |
| 421/422 | H210Q | + |
| 377/378 | I182E | + |
| 423/424 | Y93I | + |
| 425/426 | A86S | + |
| 427/428 | A68Q | + |
| 429/430 | V88L | + |
| 431/432 | Y93F | + |
| 433/434 | G200E | + |
| 435/436 | H210V | + |
| 437/438 | V83M | + |
| 439/440 | H103Y | + |
| 441/442 | H210T | + |
| 443/444 | A68R | + |
| 445/446 | A91E | + |
| 447/448 | V169I | + |

Levels of increased activity were determined relative to the reference polypeptide of SEQ ID NO: 142 and defined as follows: “+” 1.1 to 1.6, “++” 1.6 to 2.0, “+++” 2.0 to 3.3.

### Example 18

### Improvements Over SEQ ID NO: 372 in Conversion to Tetraphosphorylated Nucleotide

### HTP Screening for Improved 3OK Variants

SEQ ID NO: 372 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 18.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled oligo as described in Table 18.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 18.1
All lysis, reaction, quench, and analytical properties

Lysis conditions: Lysis buffer - 50 mM Tris-HCl buffer, pH 7.5, 0.1 g/L lysozyme; Lysis buffer
volume - 200 μL; Lysate pre-treatment - Lysates were heat-treated at 55° C. and processed as
described in Example 15. The heat-treated and clarified HTP lysates were used in reactions.
Reaction conditions: Substrate - 10 mM GTP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
18 hours
TdT coupling reaction conditions: Substrate - 10 μM SEQ ID NO: 2129, 10 μM previous reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 372 (Activity FIOP) was calculated as the fold-improvement of conversion of the variant divided by the conversion observed in the reaction with SEQ ID NO: 142 (where the conversion may be set as the average of replicates or else the highest single sample as appropriate). Conversion was calculated as 3′PO4 product (SEQ ID NO: 2132) peak areas over the total of the unreacted substrate, byproduct, and 3′PO4 product (SEQ ID NO: 2132) peak areas. The results are shown in Table 18.2.

TABLE 18.2
Relative to SEQ ID NO: 372

| SEQ ID | | Activity FIOP |
|---|---|---|
| NO: | Amino Acid Differences | Conversion Relative to |
| (nt/aa) | (Relative to SEQ ID NO: 372) | SEQ ID NO: 372 |
| 449/450 | E13A/V40M/A68Q/V74R/Q93L/ | +++ |
| | A157K | |
| 451/452 | E13A/V40M/A68R/A157K | + |
| 453/454 | V40M/A68R/R81L | + |

Levels of increased activity were determined relative to the reference polypeptide of SEQ ID NO: 372 and defined as follows: “+” 1.1 to 1.6, “++” 1.6 to 2.3, “+++” 2.3 to 2.38.

### Example 19

### Improvements Over SEQ ID NO: 450 in Conversion to Tetraphosphorylated Nucleotide

### HTP Screening for Improved 3OK Variants

SEQ ID NO: 450 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 19.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 19.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 19.1
All lysis, reaction, quench, and analytical properties

Lysis conditions: Lysis buffer - 50 mM Tris-HCl buffer, pH 7.5, 0.1 g/L lysozyme; Lysis buffer
volume - 200 μL; Lysate pre-treatment - Lysates were heat-treated at 65° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
2.25 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 450 (Activity FIOP) was calculated as the fold-improvement of conversion of the variant divided by the conversion observed in the reaction with SEQ ID NO: 450 (where the conversion may be set as the average of replicates or else the highest single sample as appropriate). Conversion was calculated as 3′ P4O product (SEQ ID NO: 2131) peak areas over the total of the unreacted substrate, byproduct, and 3′PO4 product (SEQ ID NO: 2131) peak areas. The results are shown in Table 19.2.

TABLE 19.2
Relative to SEQ ID NO: 450

| SEQ ID | | Activity FIOP |
|---|---|---|
| NO: | Amino Acid Differences | Conversion Relative to |
| (nt/aa) | (Relative to SEQ ID NO: 450) | SEQ ID NO: 450 |
| 455/456 | A72Q/K82E/A91E/T124S/A166E/ | +++ |
| | I182E | |
| 457/458 | R48H/V135D/G200E | +++ |
| 459/460 | A91E/T124Q/A166E/I182E/K201N | +++ |
| 461/462 | K82E/V88L/A91E/I182E | +++ |
| 463/464 | K82T | +++ |
| 465/466 | V135D/G200E | +++ |
| 467/468 | R48H/V135D | +++ |
| 469/470 | K82E/A166E/I182E | ++ |
| 471/472 | R81L/V135D/G200D | ++ |
| 473/474 | H103I/V135D | ++ |
| 475/476 | K82T/V88L/T124Q/A166E | ++ |
| 477/478 | H103M | ++ |
| 479/480 | R48H/V135D/V175W | ++ |
| 481/482 | V135D | ++ |
| 483/484 | H103L/V135D/G200C | ++ |
| 485/486 | R48H/H103L/G200E | ++ |
| 487/488 | R48H/R81L | ++ |
| 489/490 | R81L/V135D | ++ |
| 491/492 | R48H/H103I/G200N | + |
| 493/494 | H103L | + |
| 495/496 | R41K/A86S/T181N/A191D | + |
| 497/498 | H103F/G200N | + |
| 499/500 | I182E | + |
| 501/502 | R81L/H103M | + |
| 503/504 | R41V/A46T/L190V/A191D | + |
| 505/506 | V175W/G200D | + |
| 507/508 | R81L/G200C | + |
| 509/510 | G200D | + |
| 511/512 | R81L/H103L | + |
| 513/514 | R48H/H103V/V175W/G200C | + |
| 515/516 | K82L/V88M/A91E/T124Q/A166E/ | + |
| | I182E | |
| 517/518 | K82M/T124Q/A166E | + |
| 519/520 | T124Q/A166E/I182E | + |
| 521/522 | R48H | + |
| 523/524 | A166E/I182E | + |
| 525/526 | R48H/H103L/G200C | + |
| 527/528 | R81L/H103I | + |
| 529/530 | V135D/V175W/G200N | + |
| 531/532 | R81L/G200D | + |
| 533/534 | K82L/A166E/I182E | + |
| 535/536 | R48H/R81L/H103L | + |
| 537/538 | G200C | + |
| 539/540 | H103I/V175W | + |
| 541/542 | T124Q/A166I | + |
| 543/544 | G2009 | + |
| 545/546 | A72H/A166I/I182E | + |
| 547/548 | H103I | + |
| 549/550 | K82L/T124S/A166I/I182E | + |
| 551/552 | R41V/V83M/A86S/T181N/L190V/ | + |
| | A191D | |
| 553/554 | R41V/T181N/A191D | + |
| 555/556 | A86S | + |
| 557/558 | A91E | + |

Levels of increased activity were determined relative to the reference polypeptide of SEQ ID NO: 450 and defined as follows: “+” 1.1 to 1.3, “++” 1.3 to 1.6, “+++” 1.6 to 2.1.

### Example 20

### Improvements Over SEQ ID NO: 450 in Conversion to Tetraphosphorylated Nucleotide

### HTP Screening for Improved 3OK Variants

SEQ ID NO: 450 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 20.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 20.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 20.1
All lysis, reaction, quench, and analytical properties

Lysis conditions: Lysis buffer - 50 mM Tris-HCl buffer, pH 7.5, 0.1 g/L lysozyme; Lysis buffer
volume - 200 μL; Lysate pre-treatment - Lysates were heat-treated at 50° C. and processed as
described in Example 15. The heat-treated and clarified HTP lysates were used in reactions.
Reaction conditions: Substrate - 10 mM GTP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 450 (Activity FIOP) was calculated as the fold-improvement of conversion of the variant divided by the conversion observed in the reaction with SEQ ID NO: 450 (where the conversion may be set as the average of replicates or else the highest single sample as appropriate). Conversion was calculated as 3′PO4 product (SEQ ID NO: 2132) peak areas over the total of the unreacted substrate, byproduct, and 3′PO4 product (SEQ ID NO: 2132) peak areas. The results are shown

TABLE 20.2
Relative to SEQ ID NO: 450

| SEQ ID | | Activity FIOP |
|---|---|---|
| NO: | Amino Acid Differences | Conversion Relative to |
| (nt/aa) | (Relative to SEQ ID NO: 450) | SEQ ID NO: 450 |
| 485/486 | R48H/H103L/G200E | ++ |
| 487/488 | R48H/R81L | +++ |
| 491/492 | R48H/H103I/G200N | + |
| 493/494 | H103L | + |
| 495/496 | R41K/A86S/T181N/A191D | + |
| 497/498 | H103F/G200N | + |
| 501/502 | R81L/H103M | +++ |
| 507/508 | R81L/G200C | +++ |
| 509/510 | G200D | + |
| 511/512 | R81L/H103L | +++ |
| 521/522 | R48H | +++ |
| 525/526 | R48H/H103L/G200C | ++ |
| 527/528 | R81L/H103I | +++ |
| 531/532 | R81L/G200D | +++ |
| 535/536 | R48H/R81L/H103L | ++ |
| 537/538 | G200C | + |
| 539/540 | H103I/V175W | ++ |
| 541/542 | T124Q/A166I | + |
| 543/544 | G200N | ++ |
| 545/546 | A72H/A166I/I182E | + |
| 547/548 | H103I | + |
| 559/560 | R81L/V175W/G200E | +++ |
| 561/562 | R81L | +++ |
| 563/564 | R81L/V175W/G200N | ++ |
| 565/566 | A72H/K82E/A91E | ++ |
| 567/568 | A72H/K82T/V88L/T124Q/A166E | ++ |
| 569/570 | R48H/G200N | ++ |
| 571/572 | R41K/K95I/V111I/T181N/L190V/ | + |
| | A191D | |
| 573/574 | A72H/I182E | + |
| 575/576 | T124S | + |
| 577/578 | A46T/V83M/L190V/A191D | + |
| 579/580 | V175W | + |
| 581/582 | A72H | + |
| 583/584 | A72H/A166E | + |
| 585/586 | H103L/V175W/G200D | + |
| 587/588 | R41K/A46T/A86S/K95I/V111I | + |
| 589/590 | K82L/T124Q/A166L | + |
| 591/592 | A72H/K82L/A166I | + |
| 593/594 | A166L | + |
| 595/596 | A46T/L190V/A191D | + |
| 597/598 | R41K/L190V | + |
| 599/600 | A91E/A166E | + |
| 601/602 | R41K/A86S/T181N/L190V/A191D | + |
| 603/604 | A46T/A86S/T181N/L190V/A191D | + |
| 605/606 | V88L/A166E | + |
| 607/608 | R41K/A46T/A86S/K95L/A191D | + |
| 609/610 | A72H/T124Q/A166I | + |
| 611/612 | R48H/R81L/H103L/V175W/G200C | + |

Levels of increased activity were determined relative to the reference polypeptide of SEQ ID NO: 450 and defined as follows: “+” 1.1 to 1.5, “++” 1.5 to 1.9, “+++” 1.9 to 2.5.

### Example 21

### Improvements Over SEQ ID NO: 450 in Conversion to Tetraphosphorylated Nucleotide

### HTP Screening for Improved 3OK Variants

SEQ ID NO: 450 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 21.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 21.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 21.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 50° C. and processed as
described in Example 15. The heat-treated and clarified HTP lysates were used in reactions.
Reaction conditions: Substrate - 10 mM GTP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 450 (Activity FIOP) was calculated as the fold-improvement of conversion of the variant divided by the conversion observed in the reaction with SEQ ID NO: 450 (where the conversion may be set as the average of replicates or else the highest single sample as appropriate). Conversion was calculated as 3′PO4 product (SEQ ID NO: 2132) peak areas over the total of the unreacted substrate, byproduct, and 3′PO4 product (SEQ ID NO: 2132) peak areas. The results are shown in Table 21.2.

TABLE 21.2
Relative to SEQ ID NO: 450

| SEQ ID | | Activity FIOP |
|---|---|---|
| NO: | Amino Acid Differences | Conversion Relative to |
| (nt/aa) | (Relative to SEQ ID NO: 450) | SEQ ID NO: 450 |
| 613/614 | W126L | +++ |
| 615/616 | F3V | ++ |
| 617/618 | A61E | ++ |
| 619/620 | R171M | + |
| 621/622 | W126C | + |
| 623/624 | R105K | + |
| 625/626 | R142K | + |
| 627/628 | W126V | + |
| 629/630 | H125Q | + |

Levels of increased activity were determined relative to the reference polypeptide of SEQ ID NO: 450 and defined as follows: “+” 1.0 to 1.3, “++” 1.3to 1.7, “+++” 1.7 to 2.1.

### Example 22

### Improvements Over SEQ ID NO: 450 in Conversion to Tetraphosphorylated Nucleotide

### HTP Screening for Improved 3OK Variants

SEQ ID NO: 450 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 22.1.

Reactions were performed at 2 μL scale in 96-well format 200 μL BioRad PCR plates. Reactions included substrate, LiKAcPO₄, MgCl₂, AcK101, 50% v/v lysate, in Tris-HC. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 22.1. The reaction plates were heat-sealed with a peelable aluminum seal and incubated at 30 DC for 1 h and 95° C. for 2 min, then held at 4 NC until the reaction was quenched.

Reactions were quenched and analyzed by CE analysis as described in Example 14.

TABLE 22.1
All lysis, reaction, quench, and analytical properties

Lysis conditions: Lysis buffer - 50 mM Tris-HCl buffer, pH 7.5, 0.1 g/L lysozyme; Lysis buffer volume - 400
μL; Lysate pre-treatment - Lysates were heat-treated at 50° C. and processed as described in Example 15. The
heat-treated and clarified HTP lysates were used in reactions.
Reaction conditions: Substrate - 1 mM CTP; Reaction buffer - 50 mM Tris-HCl, pH 7.5, 50 mM LiKAcPO_{4,} 10
mM Magnesium chloride, 0.2 g/L AcK101; Lysate dilution - None; Reaction time - 16 hour
TdT coupling reaction conditions: Substrate - 10 μM SEQ ID NO: 2129, 10 μM above reaction mixture; Reaction
buffer - 20 mM TEoA, pH 7.8, 50 mM CoCl₂, 0.001 U/μL IPP; Enzyme: 4 μM SEQ ID NO: 2125

Activity relative to SEQ ID NO: 450 (Activity FIOP) was calculated as the fold-improvement of conversion of the variant divided by the conversion observed in the reaction with SEQ ID NO: 450 (where the conversion may be set as the average of replicates or else the highest single sample as appropriate). Conversion was calculated as 3′ P04 product (SEQ ID NO: 2133) peak areas over the total of the unreacted substrate, byproduct, and 3′PO4 product (SEQ ID NO: 2133) peak areas. The results are shown in Table 22.2.

TABLE 22.2
Relative to SEQ ID NO: 450

| SEQ ID | | Activity FIOP |
|---|---|---|
| NO: | Amino Acid Differences | Conversion Relative to |
| (nt/aa) | (Relative to SEQ ID NO: 450) | SEQ ID NO: 450 |
| 621/622 | W126C | + |
| 631/632 | A36R | +++ |
| 633/634 | W126A | +++ |
| 635/636 | I50M | +++ |
| 637/638 | N85R | +++ |
| 639/640 | N85L | +++ |
| 641/642 | E78A | ++ |
| 643/644 | G122A | ++ |
| 645/646 | G203C | ++ |
| 647/648 | K100W | ++ |
| 649/650 | N85A | ++ |
| 651/652 | A191C | ++ |
| 653/654 | F3C | ++ |
| 655/656 | S204A | ++ |
| 657/658 | L97A | ++ |
| 659/660 | A32G | ++ |
| 661/662 | F3D | ++ |
| 663/664 | I58L | ++ |
| 665/666 | L7I | ++ |
| 667/668 | L25A | ++ |
| 669/670 | A2L | + |
| 671/672 | E78M | + |
| 673/674 | W126G | + |
| 675/676 | K170A | + |
| 677/678 | K201C | + |
| 679/680 | P28T | + |
| 681/682 | N85G | + |
| 683/684 | V51L | + |
| 685/686 | F3K | + |
| 687/688 | E44C | + |
| 689/690 | G57A | + |
| 691/692 | S121L | + |
| 693/694 | P28V | + |
| 695/696 | L7M | + |
| 697/698 | Q49G | + |
| 699/700 | H125R | + |
| 701/702 | I58C | + |
| 703/704 | G63E | + |
| 705/706 | M22A | + |
| 707/708 | R105A | + |
| 709/710 | A35M | + |
| 711/712 | I104T | + |
| 713/714 | G202D | + |
| 715/716 | K100R | + |
| 717/718 | E167D | + |
| 719/720 | H125P | + |
| 721/722 | S19G | + |
| 723/724 | S19C | + |
| 725/726 | A32W | + |
| 727/728 | D77N | + |
| 729/730 | H125C | + |
| 731/732 | S204T | + |
| 733/734 | A98I | + |
| 735/736 | S204L | + |
| 737/738 | V29C | + |
| 739/740 | H125M | + |
| 741/742 | D136A | + |
| 743/744 | K201A | + |
| 745/746 | Q49H | + |
| 747/748 | L97N | + |
| 749/750 | E153D | + |
| 751/752 | E153V | + |
| 753/754 | F3L | + |
| 755/756 | Q49M | + |

Levels of increased activity were determined relative to the reference polypeptide of SEQ ID NO: 450 and defined as follows: “+” 1.12 to 1.17, “++” 1.17 to t 1.24, “+++” 1.24 to 1.44.

### Example 23

### Improvements Over SEQ ID NO: 450 in Conversion to Tetraphosphorylated Nucleotide

### HTP Screening for Improved 3OK Variants

SEQ ID NO: 450 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 23.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 23.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 23.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 65° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
2 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 450 (Activity FIOP) was calculated as the fold-improvement of conversion of the variant divided by the conversion observed in the reaction with SEQ ID NO: 450 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 23.2.

TABLE 23.2
Relative to SEQ ID NO: 450

| SEQ ID | | Activity FIOP |
|---|---|---|
| NO: | Amino Acid Differences | Conversion Relative to |
| (nt/aa) | (Relative to SEQ ID NO: 450) | SEQ ID NO: 450 |
| 613/614 | W126L | ++ |
| 615/616 | F3V | + |
| 617/618 | A61E | + |
| 621/622 | W126C | + |
| 623/624 | R105K | + |
| 627/628 | W126V | ++ |
| 629/630 | H125Q | + |
| 633/634 | W126A | + |
| 635/636 | I50M | + |
| 653/654 | F3C | + |
| 655/656 | S204A | + |
| 659/660 | A32G | + |
| 665/666 | L7I | + |
| 669/670 | A2L | + |
| 673/674 | W126G | + |
| 679/680 | P28T | + |
| 683/684 | V51L | + |
| 699/700 | H125R | ++ |
| 705/706 | M22A | + |
| 711/712 | I104T | + |
| 713/714 | G202D | ++ |
| 719/720 | H125P | + |
| 725/726 | A32W | +++ |
| 727/728 | D77N | + |
| 729/730 | H125C | +++ |
| 731/732 | S204T | + |
| 733/734 | A98I | + |
| 737/738 | V29C | +++ |
| 747/748 | L97N | +++ |
| 753/754 | F3L | ++ |
| 757/758 | A13D | +++ |
| 759/760 | R142D | +++ |
| 761/762 | A53K | +++ |
| 763/764 | R105L | +++ |
| 765/766 | K170P | +++ |
| 767/768 | A36S | +++ |
| 769/770 | A36G | +++ |
| 771/772 | W196Y | +++ |
| 773/774 | Y47S | +++ |
| 775/776 | Q195P | +++ |
| 777/778 | D173Y | +++ |
| 779/780 | A53E | +++ |
| 781/782 | L199C | ++ |
| 783/784 | Q79G | ++ |
| 785/786 | Q49C | ++ |
| 787/788 | P28D | ++ |
| 789/790 | S204C | ++ |
| 791/792 | K109S | ++ |
| 793/794 | G122I | ++ |
| 795/796 | L25M | ++ |
| 797/798 | R171S | ++ |
| 799/800 | I58T | ++ |
| 801/802 | K201S | ++ |
| 803/804 | E44A | ++ |
| 805/806 | E167T | ++ |
| 807/808 | A53F | ++ |
| 809/810 | F3P | ++ |
| 811/812 | K94A | ++ |
| 813/814 | I50S | ++ |
| 815/816 | L59S | ++ |
| 817/818 | H125Y | ++ |
| 819/820 | T17S | ++ |
| 821/822 | K170G | ++ |
| 823/824 | E152D | ++ |
| 825/826 | Y47K | ++ |
| 827/828 | P28N | ++ |
| 829/830 | G202P | ++ |
| 831/832 | Q49V | ++ |
| 833/834 | K129S | ++ |
| 835/836 | G202I | ++ |
| 837/838 | D197V | ++ |
| 839/840 | K15S | ++ |
| 841/842 | K100S | ++ |
| 843/844 | R105E | ++ |
| 845/846 | N85P | + |
| 847/848 | R142Y | + |
| 849/850 | K94C | + |
| 851/852 | E44D | + |
| 853/854 | T4G | + |
| 855/856 | R171G | + |
| 857/858 | G63S | + |
| 859/860 | R105S | + |
| 861/862 | G57C | + |
| 863/864 | R105G | + |
| 865/866 | G149P | + |
| 867/868 | I50E | + |
| 869/870 | T4V | + |
| 871/872 | R171L | + |
| 873/874 | G55Y | + |
| 875/876 | M22S | + |
| 877/878 | R60V | + |
| 879/880 | D173P | + |
| 881/882 | G202T | + |
| 883/884 | V127C | + |
| 885/886 | E152I | + |
| 887/888 | L199V | + |
| 889/890 | V92T | + |
| 891/892 | G6W | + |
| 893/894 | A2V | + |
| 895/896 | Q195S | + |
| 897/898 | H194G | + |
| 899/900 | G203R | + |
| 901/902 | G26T | + |
| 903/904 | R171T | + |
| 905/906 | V139A | + |
| 907/908 | G203L | + |
| 909/910 | L84P | + |
| 911/912 | A61Y | + |
| 913/914 | A53R | + |
| 915/916 | L7S | + |
| 917/918 | R60L | + |
| 919/920 | R171V | + |
| 921/922 | I5T | + |
| 923/924 | W126M | + |
| 925/926 | A32K | + |
| 927/928 | N85V | + |
| 929/930 | I104C | + |
| 931/932 | I104R | + |
| 933/934 | K109L | + |
| 935/936 | L25V | + |
| 937/938 | G149T | + |
| 939/940 | P56M | + |
| 941/942 | E167I | + |
| 943/944 | I50V | + |
| 945/946 | A191T | + |
| 947/948 | G203A | + |
| 949/950 | H194E | + |
| 951/952 | E44G | + |
| 953/954 | K100I | + |
| 955/956 | V127Y | + |
| 957/958 | K170I | + |
| 959/960 | I5Y | + |
| 961/962 | G149F | + |
| 963/964 | V51T | + |
| 965/966 | G57W | + |
| 967/968 | I104Y | + |
| 969/970 | Y47G | + |
| 971/972 | E101I | + |
| 973/974 | A52I | + |
| 975/976 | G63T | + |
| 977/978 | R60C | + |
| 979/980 | W126P | + |
| 981/982 | I104A | + |
| 983/984 | D197K | + |
| 985/986 | A35S | + |
| 987/988 | G6F | + |
| 989/990 | R105I | + |
| 991/992 | I104G | + |
| 993/994 | R105M | + |
| 995/996 | K170Y | + |
| 997/998 | S204R | + |
| 999/1000 | G149L | + |
| 1001/1002 | D136F | + |
| 1003/1004 | L199T | + |
| 1005/1006 | G6C | + |
| 1007/1008 | G149K | + |
| 1009/1010 | P56E | + |
| 1011/1012 | D173H | + |
| 1013/1014 | K170C | + |
| 1015/1016 | E153S | + |
| 1017/1018 | M22E | + |
| 1019/1020 | E44P | + |
| 1021/1022 | D173V | + |
| 1023/1024 | K109R | + |
| 1025/1026 | G63I | + |

Levels of increased activity were determined relative to the reference polypeptide of SEQ ID NO: 450 and defined as follows: “+” 1.1 to 1.2, “++” 1.2 to 1.5, “+++” 1.5 to 3.1.

### Example 24

### Improvements Over SEQ ID NO: 496 in Conversion to Tetraphosphorylated Nucleotide

### HTP Screening for Improved 3OK Variants

SEQ ID NO: 496 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 24.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 24.1. The reaction plates were heat-sealed with a peelable aluminum seal and incubated at 30° C. for 1 h and 95° C. for 2 min, then held at 4 RC until the reaction was quenched.

Reactions were quenched and analyzed by CE analysis as described in Example 14.

TABLE 24.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 63° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 496 (Activity FIOP) was calculated as the fold-improvement of conversion of the variant divided by the conversion observed in the reaction with SEQ ID NO: 496 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 24.2.

TABLE 24.2
Relative to SEQ ID NO: 496

| SEQ ID | | Activity FIOP |
|---|---|---|
| NO: | Amino Acid Differences | Conversion Relative to |
| (nt/aa) | (Relative to SEQ ID NO: 496) | SEQ ID NO: 496 |
| 1027/1028 | S86A/W126L/V135D/R142K | +++ |
| 1029/1030 | F3V/Q49D/A61E/T124Q/H125Q/R171M/G200D | +++ |
| 1031/1032 | I50V/A91E/W126V/V135D | +++ |
| 1033/1034 | F3V/Q49D/A61E/R81L/T124Q/H125Q/G200D | +++ |
| 1035/1036 | F3V/Q49D/A61E/R81L/T124Q/H125S/G200D | +++ |
| 1037/1038 | A72Q/S86A/A91E/L97N/V135D | +++ |
| 1039/1040 | A61E/T124S/H125S/A166E/R171M/G200D | +++ |
| 1041/1042 | F3V/Q49D/R105K/T124Q/H125S/G200D | ++ |
| 1043/1044 | F3V/Q49D/T124Q/H125Q/A166I/R171M/G200D | ++ |
| 1045/1046 | F3V/A61E/R81L/H125S/A166I/R171M/G200D | ++ |
| 1047/1048 | F3V/A61E/R81L/R105K/T124Q/H125S/A166I/R171M | ++ |
| 1049/1050 | A61E/V83M/T124Q/H125S/G200D | ++ |
| 1051/1052 | A61E/R81L/H125S/A166E/R171M/G200D | ++ |
| 1053/1054 | K94A/W126L | ++ |
| 1055/1056 | Q49D/V111I/T124Q/A166E/R171M/G200D | ++ |
| 1057/1058 | Q49D/A61E/T124Q/H125S/A166I/R171M | ++ |
| 1059/1060 | F3V/Q49D/A61E/V83M/G200D | ++ |
| 1061/1062 | S86A/K94A/V111I/W126L/R142K | ++ |
| 1063/1064 | A61E/G200D | + |
| 1065/1066 | F3V/R81L/T124S/H125S/G200D | + |
| 1067/1068 | Q49D/A61E/V83M/R105K/T124S/H125S/R171M | + |
| 1069/1070 | A61E/R81L/V83M/A166I/R171M/G200D | + |
| 1071/1072 | F3V/Q49D/R81L/T124Q/H125Q/A166E/R171M | + |
| 1073/1074 | F3V/Q49D/A61E/V83M/H125S/A166E | + |
| 1075/1076 | A61E/H125Q | + |
| 1077/1078 | F3V/A166E/R171M | + |
| 1079/1080 | Q49D/A61E/H125S/R171M | + |
| 1081/1082 | K82E/V83M | + |
| 1083/1084 | F3V/R81L/R105K/T124Q/A166I | + |
| 1085/1086 | A61E/A166E | + |
| 1087/1088 | V83M/R105K/T124S/H125S/A166I/G200D | + |
| 1089/1090 | F3V/Q49D/A61E/R81L/V83M/T124S/H125S/A166I/R171M | + |
| 1091/1092 | Q49D/A61E/V83M/T124S/H125S/R171M | + |
| 1093/1094 | A72Q/R142K/I182E | + |
| 1095/1096 | Q49D/T124Q/H125Q/A166I | + |
| 1097/1098 | F3V/Q49D/R81L/V83M/T124S/H125S/G200D | + |
| 1099/1100 | W126V | + |
| 1101/1102 | F3V/Q49D/R81L/V83M/T124Q/H125S/R171M | + |
| 1103/1104 | G200D | + |
| 1105/1106 | K82E/V83M/H103F | + |
| 1107/1108 | A72Q/K82E/V83M/R142K/N181T/D191A/G200N | + |
| 1109/1110 | I182E | + |
| 1111/1112 | F3V/Q49D/A166E/R171M | + |
| 1113/1114 | F3V/Q49D/A61E/V83M | + |
| 1115/1116 | F3V/V83M/A166I/R171M | + |
| 1117/1118 | T124Q/H125S | + |
| 1119/1120 | R142K | + |
| 1121/1122 | Q49D/V83M/R105K/V111I/T124Q | + |
| 1123/1124 | Q49D/A61E/R81L/V83M | + |
| 1125/1126 | I50V/R60L/K82E/V83M/H103M/W126L/R142K/V175W/D191A | + |
| 1127/1128 | V83M/A91E/K94A/K95I/W126L/V135D/D191A | + |
| 1129/1130 | R60L/I182E | + |
| 1131/1132 | V83M/R105K/A166E | + |
| 1133/1134 | Q49D/V83M/R105K/T124Q/H125Q/A166I | + |
| 1135/1136 | R105K | + |
| 1137/1138 | V111I/W126V/V135D/V175W/I182E | + |
| 1139/1140 | V83M/H125S/R171M | + |
| 1141/1142 | I50V/R60L/A72Q/S86A/H103L | + |

Levels of increased activity were determined relative to the reference polypeptide of SEQ ID NO: 496 and defined as follows: “+” 1.1 to 2.7, “++” 2.7 to 5.0, “+++” 5.0 o 14.9.

### Example 25

### Improvements Over SEQ ID NO: 496 in Conversion to Tetraphosphorylated Nucleotide

### HTP Screening for Improved 3OK Variants

SEQ ID NO: 496 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 25.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 25.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 25.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 50° C. and processed as
described in Example 15. The heat-treated and clarified HTP lysates were used in reactions.
Reaction conditions: Substrate - 1 mM CTP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 496 (Activity FIOP) was calculated as the fold-improvement of conversion of the variant divided by the conversion observed in the reaction with SEQ ID NO: 496 (where the conversion may be set as the average of replicates or else the highest single sample as appropriate). Conversion was calculated as 3′PO4 product (SEQ ID NO: 2133) peak areas over the total of the unreacted substrate, byproduct, and 3′PO4 product (SEQ ID NO: 2133) peak areas. The results are shown in Table 25.2.

TABLE 25.2
Relative to SEQ ID NO: 496

| SEQ ID | | Activity FIOP |
|---|---|---|
| NO: | Amino Acid Differences | Conversion Relative to |
| (nt/aa) | (Relative to SEQ ID NO: 496) | SEQ ID NO: 496 |
| 1041/1042 | F3V/Q49D/R105K/T124Q/H125S/ | + |
| | G200D | |
| 1059/1060 | F3V/Q49D/A61E/V83M/G200D | + |
| 1099/1100 | W126V | ++ |
| 1107/1108 | A72Q/K82E/V83M/R142K/N181T/ | +++ |
| | D191A/G200N | |
| 1119/1120 | R142K | + |
| 1143/1144 | D191A/G200D | ++ |

Levels of increased activity were determined relative to the reference polypeptide of SEQ ID NO: 496 and defined as follows: “+” 1.06 to 1.11, “++” 1.1 to 1.15, “+++” 1.15 to 1.15.

### Example 26

### Improvements Over SEQ ID NO: 1042 in Conversion to Tetraphosphorylated Nucleotide

### HTP Screening for Improved 3OK Variants

SEQ ID NO: 1042 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 26.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 26.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14

TABLE 26.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 65° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1042 (Activity FIOP) was calculated as the fold-improvement of conversion of the variant divided by the conversion observed in the reaction with SEQ ID NO: 1042 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 26.2.

TABLE 26.2
Relative to SEQ ID NO: 1042

| SEQ ID | | Activity FIOP |
|---|---|---|
| NO: | Amino Acid Differences | Conversion Relative to |
| (nt/aa) | (Relative to SEQ ID NO: 1042) | SEQ ID NO: 1042 |
| 1145/1146 | N85A | +++ |
| 1147/1148 | K105G/K201H | +++ |
| 1149/1150 | R171M/K201S | +++ |
| 1151/1152 | A61E | +++ |
| 1153/1154 | Q79G/W126M/S204C | ++ |
| 1155/1156 | N85R/L97N | ++ |
| 1157/1158 | A35M/I50E | ++ |
| 1159/1160 | A13D | ++ |
| 1161/1162 | L7I/N85G/L97A/W126M | ++ |
| 1163/1164 | A53E/I58T/K100R/K105G/K109S | ++ |
| 1165/1166 | I50M/E78A/R142D | + |
| 1167/1168 | L7I | + |
| 1169/1170 | S19G/K100R/K105L/K201H | + |
| 1171/1172 | K100R | + |
| 1173/1174 | W126M | + |
| 1175/1176 | S19G/A53N/K105G | + |
| 1177/1178 | A53N/I58T/K109S/K201S | + |
| 1179/1180 | A53E/K100W/K105G | + |
| 1181/1182 | S204M | + |
| 1183/1184 | L97A | + |
| 1185/1186 | L7S/A61E/N85R/L97A/W126M | + |
| 1187/1188 | W126A | + |
| 1189/1190 | S19G | + |
| 1191/1192 | A32W/N85R/W126G/S204C | + |
| 1193/1194 | S19G/A53N/K201H | + |
| 1195/1196 | Q79G | + |

Levels of increased activity were determined relative to the reference polypeptide of SEQ ID NO: 1042 and defined as follows: “+” 1.1 to 1.4, “++” 1.4 to 1.7, “+++” 1.7 to 2.0.

### Example 27

### Improvements Over SEQ ID NO: 1042 in Conversion to Tetraphosphorylated Nucleotide

### HTP Screening for Improved 3OK Variants

SEQ ID NO: 1042 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 27.1.

Reactions were performed at 2 μL scale in 96-well format 200 μL BioRad PCR plates. Reactions included substrate, LiKAcPO₄, MgCl₂, Ack101, 50% v/v lysate, in Tris-HCL. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 27.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 27.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 50° C. and processed as
described in Example 15. The heat-treated and clarified HTP lysates were used in reactions.
Reaction conditions: Substrate - 1 mM UTP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1042 (Activity FIOP) was calculated as the fold-improvement of conversion of the variant divided by the conversion observed in the reaction with SEQ ID NO: 1042 (where the conversion may be set as the average of replicates or else the highest single sample as appropriate). Conversion was calculated as 3′PO4 product (SEQ ID NO: 2134) peak areas over the total of the unreacted substrate, byproduct, and 3′PO4 product (SEQ ID NO: 2134) peak areas. The results are shown in Table 27.2.

TABLE 27.2
Relative to SEQ ID NO: 1042

| SEQ ID | | Activity FIOP |
|---|---|---|
| NO: | Amino Acid Differences | Conversion Relative to |
| (nt/aa) | (Relative to SEQ ID NO: 1042) | SEQ ID NO: 1042 |
| 1145/1146 | N85A | + |
| 1155/1156 | N85R/L97N | + |
| 1179/1180 | A53E/K100W/K105G | + |
| 1191/1192 | A32W/N85R/W126G/S204C | +++ |
| 1197/1198 | A36R/A61E/W126M/S204C | +++ |
| 1199/1200 | P28T/A32W/N85R | ++ |
| 1201/1202 | A32W/A36G/W126M | ++ |
| 1203/1204 | P28T/A36R/A61E | ++ |
| 1205/1206 | P28T/A32W/L97A/W126M | + |
| 1207/1208 | A32W/A36G/A61E/N85G/L97N/ | + |
| | W126M/S204C | |
| 1209/1210 | K100W/K105A | + |
| 1211/1212 | P28T/A32W/A61E | + |
| 1213/1214 | N85R/W126M | + |
| 1215/1216 | P28T/A32W/G71V/Q79G/L97A/ | + |
| | W126M/S204C | |
| 1217/1218 | K100W/K105G | + |
| 1219/1220 | L7I/P28T/A32W/L97N | + |
| 1221/1222 | L7I/A61E | + |

Levels of increased activity were determined relative to the reference polypeptide of SEQ ID NO: 1042 and defined as follows: “+” 1.1 to 1.3, “++” 1.3 to 1.6, “+++” 1.6 to 2.2.

### Example 28

### Improvements Over SEQ ID NO: 1042 in Conversion to Tetraphosphorylated Nucleotide

### HTP Screening for Improved 3OK Variants

SEQ ID NO: 1042 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 28.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 28.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 28.1
All lysis, reaction, quench, and analytical properties

Lysis conditions: Lysis buffer - 50 mM Tris-HCl buffer, pH 7.5, 0.1 g/L lysozyme; Lysis buffer
volume - 400 μL; Lysate pre-treatment - Lysates were heat-treated at 65° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
16 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1042 (Activity FIOP) was calculated as the fold-improvement of conversion of the variant divided by the conversion observed in the reaction with SEQ ID NO: 1042 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 28.2.

TABLE 28.2
Relative to SEQ ID NO: 1042

| SEQ ID | | Activity FIOP |
|---|---|---|
| NO: | Amino Acid Differences | Conversion Relative to |
| (nt/aa) | (Relative to SEQ ID NO: 1042) | SEQ ID NO: 1042 |
| 1223/1224 | R74F | +++ |
| 1225/1226 | G71A | +++ |
| 1227/1228 | A146D | +++ |
| 1229/1230 | S161V | ++ |
| 1231/1232 | I115W | ++ |
| 1233/1234 | K69R | ++ |
| 1235/1236 | I176G | ++ |
| 1237/1238 | R160L | ++ |
| 1239/1240 | I176L | ++ |
| 1241/1242 | G71I | ++ |
| 1243/1244 | I182E | ++ |
| 1245/1246 | R74I | + |
| 1247/1248 | G71T | + |
| 1249/1250 | G71Y/L131I | + |
| 1251/1252 | S165I | + |
| 1253/1254 | H103F | + |
| 1255/1256 | L141P | + |
| 1257/1258 | A146T | + |
| 1259/1260 | A146S | + |
| 1261/1262 | Y134L | + |
| 1263/1264 | Q124H | + |
| 1265/1266 | L93T | + |
| 1267/1268 | I115V | + |
| 1269/1270 | K95S | + |
| 1271/1272 | L156P | + |
| 1273/1274 | R158H | + |
| 1275/1276 | S165Q | + |
| 1277/1278 | R23A | + |
| 1279/1280 | D62G | + |
| 1281/1282 | K157M | + |
| 1283/1284 | A72N | + |
| 1285/1286 | L27F | + |
| 1287/1288 | H103V | + |
| 1289/1290 | A72D | + |
| 1291/1292 | L117G | + |
| 1293/1294 | L141I | + |
| 1295/1296 | I182C | + |
| 1297/1298 | A72T | + |
| 1299/1300 | A72K | + |
| 1301/1302 | F150L | + |
| 1303/1304 | R74G | + |
| 1305/1306 | H103M | + |
| 1307/1308 | R160D | + |
| 1309/1310 | R67F | + |
| 1311/1312 | Y134H | + |
| 1313/1314 | L27Y | + |
| 1315/1316 | A166E | + |
| 1317/1318 | A166L | + |
| 1319/1320 | K157E | + |

Levels of increased activity were determined relative to the reference polypeptide of SEQ ID NO: 1042 and defined as follows: “+” 1.1 to 1.8, “++” 1.8 to 3.4, “+++” 3.4 to 10.79.

### Example 29

### Improvements Over SEQ ID NO: 1042 in Conversion to Tetraphosphorylated Nucleotide

### HTP Screening for Improved 3OK Variants

SEQ ID NO: 1042 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 29.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 29.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 29.1
All lysis, reaction, quench, and analytical properties

Lysis conditions: Lysis buffer - 50 mM TEoA buffer, pH 7.5, 0.1 g/L lysozyme; Lysis buffer
volume - 400 μL; Lysate pre-treatment - Lysates were heat-treated at 65° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
3 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1042 (Activity FIOP) was calculated as the fold-improvement of conversion of the variant divided by the conversion observed in the reaction with SEQ ID NO: 1042 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 29.2.

TABLE 29.2
Relative to SEQ ID NO: 1042

| SEQ ID | | Activity FIOP |
|---|---|---|
| NO: | Amino Acid Differences | Conversion Relative to |
| (nt/aa) | (Relative to SEQ ID NO: 1042) | SEQ ID NO: 1042 |
| 1223/1224 | R74F | +++ |
| 1225/1226 | G71A | + |
| 1243/1244 | I182E | + |
| 1245/1246 | R74I | + |
| 1247/1248 | G71T | + |
| 1251/1252 | S165I | ++ |
| 1267/1268 | I115V | + |
| 1275/1276 | S165Q | + |
| 1281/1282 | K157M | + |
| 1285/1286 | L27F | + |
| 1289/1290 | A72D | ++ |
| 1301/1302 | F150L | ++ |
| 1305/1306 | H103M | + |
| 1321/1322 | R74C | +++ |
| 1323/1324 | R74V | +++ |
| 1325/1326 | R74A | +++ |
| 1327/1328 | G71Q | ++ |
| 1329/1330 | H103L | + |
| 1331/1332 | Q68G | + |
| 1333/1334 | A20T | + |
| 1335/1336 | Q124Y | + |
| 1337/1338 | M96I | + |
| 1339/1340 | G71R | + |
| 1341/1342 | I192L | + |
| 1343/1344 | N181T | + |
| 1345/1346 | T110A | + |
| 1347/1348 | G89L | + |
| 1349/1350 | R160E | + |
| 1351/1352 | K41A | + |
| 1353/1354 | G71V | + |

Levels of increased activity were determined relative to the reference polypeptide of SEQ ID NO: 1042 and defined as follows: “+” 1.1 to 1.5, “++” 1.5 to 1.9, “+++” 1.9 to 2.9.

### Example 30

### Improvements Over SEQ ID NO: 1042 in Conversion to Tetraphosphorylated Nucleotide

### HTP Screening for Improved 3OK Variants

SEQ ID NO: 1042 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 30.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 30.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 30.1
All lysis, reaction, quench, and analytical properties

Lysis conditions: Lysis buffer - 50 mM TEoA buffer, pH 7.5, 0.1 g/L lysozyme; Lysis buffer
volume - 400 μL; Lysate pre-treatment - Lysates were heat-treated at 55° C. and processed as
described in Example 15. The heat-treated and clarified HTP lysates were used in reactions.
Reaction conditions: Substrate - 1 mM fATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
3 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1042 (Activity FIOP) was calculated as the fold-improvement of conversion of the variant divided by the conversion observed in the reaction with SEQ ID NO: 1042 (where the conversion may be set as the average of replicates or else the highest single sample as appropriate). Conversion was calculated as 3′P04 product (SEQ ID NO: 2135) peak areas over the total of the unreacted substrate, byproduct, and 3′PO4 product (SEQ ID NO: 2135) peak areas. The results are shown in Table 30.2.

TABLE 30.2
Relative to SEQ ID NO: 1042

| SEQ ID | | Activity FIOP |
|---|---|---|
| NO: | Amino Acid Differences | Conversion Relative to |
| (nt/aa) | (Relative to SEQ ID NO: 1042) | SEQ ID NO: 1042 |
| 1229/1230 | S161V | ++ |
| 1231/1232 | I115W | + |
| 1239/1240 | I176L | + |
| 1253/1254 | H103F | ++ |
| 1291/1292 | L117G | + |
| 1303/1304 | R74G | + |
| 1333/1334 | A20T | + |
| 1355/1356 | R160K | +++ |
| 1357/1358 | W163V | ++ |
| 1359/1360 | H103C | ++ |
| 1361/1362 | Q124A | ++ |
| 1363/1364 | A20C | ++ |
| 1365/1366 | K95Y | ++ |
| 1367/1368 | V130Q | + |
| 1369/1370 | V39C | + |
| 1371/1372 | V175W | + |
| 1373/1374 | I11K | + |
| 1375/1376 | R160S | + |
| 1377/1378 | A146H | + |
| 1379/1380 | E64R | + |
| 1381/1382 | V135F | + |
| 1383/1384 | K95H | + |
| 1385/1386 | E64V | + |
| 1387/1388 | A72L | + |
| 1389/1390 | N148V | + |
| 1391/1392 | I182P | + |
| 1393/1394 | N181V | + |
| 1395/1396 | G10S | + |
| 1397/1398 | M96T | + |
| 1399/1400 | N178M | + |
| 1401/1402 | A38S | + |

Levels of increased activity were determined relative to the reference polypeptide of SEQ ID NO: 1042 and defined as follows: “+” 1.1 to 1.3, “++” 1.3 to 1.6, “+++” 1.6 to 3.0.

### Example 31

### Improvements Over SEQ ID NO: 1180 in Conversion to Tetraphosphorylated Nucleotide

### HTP Screening for Improved 3OK Variants

SEQ ID NO: 1180 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 31.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 31.1. The reaction plates were heat-sealed with a peelable aluminum seal and incubated at 30° C. for 1 h and 95 sC for 2 min, then held at 4 LC until the reaction was quenched.

Reactions were quenched and analyzed by CE analysis as described in Example 14.

TABLE 31.1
All lysis, reaction, quench, and analytical properties

Lysis conditions: Lysis buffer - 50 mM TEoA buffer, pH 7.5, 0.1 g/L lysozyme; Lysis buffer
volume - 400 μL; Lysate pre-treatment - Lysates were heat-treated at 66° C. and processed as
described in Example 15. The heat-treated and clarified HTP lysates were used in reactions.
Reaction conditions: Substrate - 10 mM ATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
3 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1180 (Activity FIOP) was calculated as the fold-improvement of conversion of the variant divided by the conversion observed in the reaction with SEQ ID NO: 1180 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 31.2.

TABLE 31.2
Relative to SEQ ID NO: 1180

| SEQ ID | | Activity FIOP |
|---|---|---|
| NO: | Amino Acid Differences | Conversion Relative to |
| (nt/aa) | (Relative to SEQ ID NO: 1180) | SEQ ID NO: 1180 |
| 1403/1404 | I58L/A61E/W126M | +++ |
| 1405/1406 | V3D/L25A/W126M | +++ |
| 1407/1408 | E167D/R171L/D173A | +++ |
| 1409/1410 | L25A/I58L/R60L | ++ |
| 1411/1412 | V127M | ++ |
| 1413/1414 | L25M/E44C/I58C | ++ |
| 1415/1416 | V3D/L25A/V29A/R60C/K170A | + |
| 1417/1418 | V3D/L25A/V29A/W126V | + |
| 1419/1420 | V3D/E44C/W126M/K170A | + |
| 1421/1422 | K170A | + |
| 1423/1424 | E44D/I58L/R60L/A61E/W126C/ | + |
| | K170A | |

Levels of increased activity were determined relative to the reference polypeptide of SEQ ID NO: 1180 and defined as follows: “+” 1.10 to 1.17, “++” 1.17 to 1.20, “+++” 1.20 to 1.21.

### Example 32

### Improvements Over SEQ ID NO: 1180 in Conversion to Tetraphosphorylated Nucleotide

### HTP Screening for Improved 3OK Variants

SEQ ID NO: 1180 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 32.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 32.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 32.1
All lysis, reaction, quench, and analytical properties

Lysis conditions: Lysis buffer - 50 mM TEoA buffer, pH 7.5, 0.1 g/L lysozyme; Lysis buffer
volume - 400 μL; Lysate pre-treatment - Lysates were heat-treated at 55° C. and processed as
described in Example 15. The heat-treated and clarified HTP lysates were used in reactions.
Reaction conditions: Substrate - 1 mM fATP; Reaction buffer - 50 mM Tris-HCl, pH 7.5, 50 mM
LiKAcPO_{4,} 10 mM Magnesium chloride, 0.2 g/L AcK101; Lysate dilution - None; Reaction time -
3 hour
TdT coupling reaction conditions: Substrate - 10 μM SEQ ID NO: 2129, 10 μM above reaction
mixture; Reaction buffer - 20 mM TEoA, pH 7.8, 50 mM CoCl₂, 0.001 U/μL IPP; Enzyme: 4 μM
SEQ ID NO: 2125

Activity relative to SEQ ID NO: 1180 (Activity FIOP) was calculated as the fold-improvement of conversion of the variant divided by the conversion observed in the reaction with SEQ ID NO: 1180 (where the conversion may be set as the average of replicates or else the highest single sample as appropriate). Conversion was calculated as 3′P04 product (SEQ ID NO: 2135) peak areas over the total of the unreacted substrate, byproduct, and 3′PO4 product (SEQ ID NO: 2135) peak areas. The results are shown in Table 32.2.

TABLE 32.2
Relative to SEQ ID NO: 1180

| SEQ ID | | Activity FIOP |
|---|---|---|
| NO: | Amino Acid Differences | Conversion Relative |
| (nt/aa) | (Relative to SEQ ID NO: 1180) | to SEQ ID NO: 1180 |
| 1411/1412 | V127M | + |
| 1425/1426 | T17S/G63S/I104F/S125R | +++ |
| 1427/1428 | T17S/G63E | ++ |
| 1429/1430 | P28V | ++ |
| 1431/1432 | L59S/I104F/S125R | ++ |
| 1433/1434 | M22A/G55Y/A98I/V127M | ++ |
| 1435/1436 | T17S/G63S/I104N | ++ |
| 1437/1438 | L25A/A36R/W126V | ++ |
| 1439/1440 | A98I/E167D/R171M | + |
| 1441/1442 | Q79N/S125R/K129S | + |
| 1443/1444 | E44C/R60L/A61E/W126M | + |
| 1445/1446 | M22A/G55Y/A98L/E167D/R171V/ | + |
| | D173A/D197V | |
| 1447/1448 | G63S/S125Y | + |
| 1449/1450 | L25A/V29A/R60C/W126L | + |

Levels of increased activity were determined relative to the reference polypeptide of SEQ ID NO: 1180 and defined as follows: “+” 1.03 to 1.06, “++” 1.06 to 1.10, “+++” 1.10 to 1.16.

### Example 33

### Improvements Over SEQ ID NO: 1412 in Conversion to Tetraphosphorylated Nucleotide

### HTP Screening for Improved 3OK Variants

SEQ ID NO: 1412 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 33.1.

Reactions were performed at 2 μL scale in 96-well format 200 μL BioRad PCR plates. Reactions included substrate, LiKAcPO₄, MgCl₂, AcK10m, 50% v/v lysate, in Tris-HC. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 33.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 33.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 66° C. and processed as described in Example 15.
The heat-treated and clarified HTP lysates were used in reactions.
Reaction conditions: Substrate - 10 mM ATP; Reaction buffer - 100 mM
Tris-HCl, pH 8, 50 mM LiKAcPO₄, 10 mM Magnesium chloride, 0.2 g/L
AcK101; Lysate dilution - None; Reaction time - 16 hour
TdT coupling reaction conditions: Substrate - 10 μM SEQ ID NO: 2129,
10 μM above reaction mixture; Reaction buffer - 20 mM TEoA, pH 7.8,
50 mM CoCl₂, 0.001 U/μL IPP; Enzyme: 4 μM SEQ ID NO: 2125

Activity relative to SEQ ID NO: 1412 (Activity FIOP) was calculated as the fold-improvement of conversion of the variant divided by the conversion observed in the reaction with SEQ ID NO: 1412 (where the conversion may be set as the average of replicates or else the highest single sample as appropriate). Conversion was calculated as 3′PO4 product (SEQ ID NO: 2131) peak areas over the total of the unreacted substrate, byproduct, and 3′PO4 product (SEQ ID NO: 2131) peak areas. The results are shown in Table 33.2.

TABLE 33.2
Relative to SEQ ID NO: 1412

| SEQ ID | | Activity FIOP |
|---|---|---|
| NO: | Amino Acid Differences | Conversion Relative |
| (nt/aa) | (Relative to SEQ ID NO: 1412) | to SEQ ID NO: 1412 |
| 1451/1452 | G71A/E184D | +++ |
| 1453/1454 | L27F/G71Q | +++ |
| 1455/1456 | L27F/Q68G/G71A/E184D | +++ |
| 1457/1458 | G71A | ++ |
| 1459/1460 | L27F/G71T/E184D | ++ |
| 1461/1462 | Q68G/G71R/L113M | + |
| 1463/1464 | Q68G/G71R/K157M/E184D | + |
| 1465/1466 | L27F | + |
| 1467/1468 | Q68G/G71Q/L113M/K157R/I176L | + |
| 1469/1470 | G71T/E184D | + |
| 1471/1472 | L27F/K95Y | + |
| 1473/1474 | L27F/K95H | + |
| 1475/1476 | K41A/A72D/R160S/S161V | + |
| 1477/1478 | L27F/Q68G | + |

Levels of increased activity were determined relative to the reference polypeptide of SEQ ID NO: 1412 and defined as follows: “+” 1.2 to 3.5, “++” 3.5 to 5.8, “+++” 5.8 to 8.4.

### Example 34

### Improvements Over SEQ ID NO: 1412 in Conversion to Tetraphosphorylated Nucleotide

### HTP Screening for Improved 3OK Variants

SEQ ID NO: 1412 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 34.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 34.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 34.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 55° C. and processed as described in Example 15.
The heat-treated and clarified HTP lysates were used in reactions.
Reaction conditions: Substrate - 10 mM UTP; Reaction buffer - 100 mM
Tris-HCl, pH 8, 50 mM LiKAcPO₄, 10 mM Magnesium chloride, 0.2 g/L
AcK101; Lysate dilution - None; Reaction time - 16 hour
TdT coupling reaction conditions: Substrate - 10 μM SEQ ID NO: 2129,
10 μM above reaction mixture; Reaction buffer - 20 mM TEoA, pH 7.8,
50 mM CoCl₂, 0.001 U/μL IPP; Enzyme: 4 μM SEQ ID NO: 2125

Activity relative to SEQ ID NO: 1412 (Activity FIOP) was calculated as the fold-improvement of conversion of the variant divided by the conversion observed in the reaction with SEQ ID NO: 1412 (where the conversion may be set as the average of replicates or else the highest single sample as appropriate). Conversion was calculated as 3′PO4 product (SEQ ID NO: 2134) peak areas over the total of the unreacted substrate, byproduct, and 3′PO4 product (SEQ ID NO: 2131) peak areas. The results are shown in Table 34.2.

TABLE 34.2
Relative to SEQ ID NO: 1412

| SEQ ID | | Activity FIOP |
|---|---|---|
| NO: | Amino Acid Differences | Conversion Relative |
| (nt/aa) | (Relative to SEQ ID NO: 1412) | to SEQ ID NO: 1412 |
| 1479/1480 | S125E | +++ |
| 1481/1482 | A61G | ++ |
| 1483/1484 | V83R | ++ |
| 1485/1486 | D49Q | + |
| 1487/1488 | A52S | + |
| 1489/1490 | S125A | + |
| 1491/1492 | D49M | + |

Levels of increased activity were determined relative to the reference polypeptide of SEQ ID NO: 1412 and defined as follows: “+” 1.1 to 1.3, “++” 1.3 to 1.4, “+++” 1.4 to 1.41.

### Example 35

### Improvements Over SEQ ID NO: 1412 in Conversion to Tetraphosphorylated Nucleotide

### HTP Screening for Improved 3OK Variants

SEQ ID NO: 1412 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 35.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 35.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 35.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 66° C. and processed as described in Example 15.
The heat-treated and clarified HTP lysates were used in reactions.
Reaction conditions: Substrate - 10 mM ATP; Reaction buffer - 100 mM
Tris-HCl, pH 8, 50 mM LiKAcPO₄, 10 mM Magnesium chloride, 0.2 g/L
AcK101; Lysate dilution - None; Reaction time - 16 hour
TdT coupling reaction conditions: Substrate - 10 μM SEQ ID NO: 2129,
10 μM above reaction mixture; Reaction buffer - 20 mM TEoA, pH 7.8,
50 mM CoCl₂, 0.001 U/μL IPP; Enzyme: 4 μM SEQ ID NO: 2125

Activity relative to SEQ ID NO: 1412 (Activity FIOP) was calculated as the fold-improvement of conversion of the variant divided by the conversion observed in the reaction with SEQ ID NO: 1412 (where the conversion may be set as the average of replicates or else the highest single sample as appropriate). Conversion was calculated as 3′P04 product (SEQ ID NO: 2131) peak areas over the total of the unreacted substrate, byproduct, and 3′PO4 product (SEQ ID NO: 2131) peak areas. The results are shown in Table 35.2.

TABLE 35.2
Relative to SEQ ID NO: 1412

| SEQ ID | | Activity FIOP |
|---|---|---|
| NO: | Amino Acid Differences | Conversion Relative |
| (nt/aa) | (Relative to SEQ ID NO: 1412) | to SEQ ID NO: 1412 |
| 1491/1492 | D49M | + |
| 1493/1494 | M127P | +++ |
| 1495/1496 | R186P | +++ |
| 1497/1498 | A2V | +++ |
| 1499/1500 | E101L | +++ |
| 1501/1502 | S76D | +++ |
| 1503/1504 | K82D | +++ |
| 1505/1506 | K94Q | +++ |
| 1507/1508 | R186V | +++ |
| 1509/1510 | K94N | +++ |
| 1511/1512 | T4P | +++ |
| 1513/1514 | A2W | +++ |
| 1515/1516 | A35Y | +++ |
| 1517/1518 | K94A | +++ |
| 1519/1520 | K94T | ++ |
| 1521/1522 | A2R | ++ |
| 1523/1524 | M127A | ++ |
| 1525/1526 | L97W | ++ |
| 1527/1528 | S76G | ++ |
| 1529/1530 | D49G | ++ |
| 1531/1532 | D49N | ++ |
| 1533/1534 | A13M | ++ |
| 1535/1536 | G57L | ++ |
| 1537/1538 | A35L | ++ |
| 1539/1540 | E167A | ++ |
| 1541/1542 | Q80T | ++ |
| 1543/1544 | D49V | + |
| 1545/1546 | E45L | + |
| 1547/1548 | I30W | + |
| 1549/1550 | E78V | + |
| 1551/1552 | V133N | + |
| 1553/1554 | P56L | + |
| 1555/1556 | V83E | + |
| 1557/1558 | D191G | + |
| 1559/1560 | E154Q | + |
| 1561/1562 | A35R | + |
| 1563/1564 | A52Q | + |
| 1565/1566 | S121E | + |
| 1567/1568 | R60V | + |
| 1569/1570 | A36S | + |
| 1571/1572 | K82H | + |
| 1573/1574 | I104A | + |
| 1575/1576 | A61H | + |
| 1577/1578 | E152L | + |
| 1579/1580 | K109D | + |
| 1581/1582 | V139Q | + |
| 1583/1584 | S76T | + |
| 1585/1586 | L97G | + |
| 1587/1588 | V92A | + |
| 1589/1590 | W100R | + |
| 1591/1592 | A36G | + |
| 1593/1594 | A52L | + |
| 1595/1596 | A52T | + |
| 1597/1598 | A13V | + |
| 1599/1600 | V3S | + |
| 1601/1602 | K109S | + |
| 1603/1604 | R81W | + |
| 1605/1606 | D197A | + |
| 1607/1608 | P90R | + |
| 1609/1610 | A2Q | + |
| 1611/1612 | V83L | + |
| 1613/1614 | F54M | + |
| 1615/1616 | G43E | + |
| 1617/1618 | M40R | + |
| 1619/1620 | D173A | + |
| 1621/1622 | A36E | + |
| 1623/1624 | N85R | + |
| 1625/1626 | R48V | + |
| 1627/1628 | D173I | + |
| 1629/1630 | E53G | + |
| 1631/1632 | V3G | + |
| 1633/1634 | A2L | + |
| 1635/1636 | G57K | + |
| 1637/1638 | N85M | + |
| 1639/1640 | G57V | + |
| 1641/1642 | K129P | + |
| 1643/1644 | E78G | + |
| 1645/1646 | E154T | + |
| 1647/1648 | R186A | + |
| 1649/1650 | V83Q | + |
| 1651/1652 | L97T | + |
| 1653/1654 | G63F | + |
| 1655/1656 | I104L | + |
| 1657/1658 | A52D | + |
| 1659/1660 | V139L | + |
| 1661/1662 | A32W | + |
| 1663/1664 | L198P | + |
| 1665/1666 | A36P | + |
| 1667/1668 | V3A | + |
| 1669/1670 | Q80V | + |
| 1671/1672 | R48E | + |
| 1673/1674 | W100N | + |
| 1675/1676 | L198G | + |
| 1677/1678 | G63Q | + |
| 1679/1680 | V92T | + |
| 1681/1682 | V139R | + |
| 1683/1684 | V139T | + |
| 1685/1686 | E153V | + |
| 1687/1688 | R48P | + |
| 1689/1690 | P56K | + |
| 1691/1692 | K94G | + |
| 1693/1694 | V51A | + |
| 1695/1696 | D77E | + |
| 1697/1698 | H194M | + |
| 1699/1700 | P56G | + |
| 1701/1702 | P28E | + |
| 1703/1704 | R60S | + |
| 1705/1706 | A35V | + |
| 1707/1708 | D77W | + |
| 1709/1710 | N85A | + |
| 1711/1712 | T4Q | + |
| 1713/1714 | D173P | + |
| 1715/1716 | S86Q | + |

Levels of increased activity were determined relative to the reference polypeptide of SEQ ID NO: 1412 and defined as follows: “+” 1.1 to 2.6, “++” 2.6 to 5.4, “+++” 5.4 to 21.2.

### Example 36

### Improvements Over SEQ ID NO: 1412 in Conversion to Tetraphosphorylated Nucleotide

### HTP Screening for Improved 3OK Variants

SEQ ID NO: 1412 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 36.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 36.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 36.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 55° C. and processed as described in Example 15.
The heat-treated and clarified HTP lysates were used in reactions.
Reaction conditions: Substrate - 10 mM CTP; Reaction buffer - 100 mM
Tris-HCl, pH 8, 50 mM LiKAcPO₄, 10 mM Magnesium chloride, 0.2 g/L
AcK101; Lysate dilution - None; Reaction time - 16 hour
TdT coupling reaction conditions: Substrate - 10 μM SEQ ID NO: 2129,
10 μM above reaction mixture; Reaction buffer - 20 mM TEoA, pH 7.8,
50 mM CoCl₂, 0.001 U/μL IPP; Enzyme: 4 μM SEQ ID NO: 2125

Activity relative to SEQ ID NO: 1412 (Activity FIOP) was calculated as the fold-improvement of conversion of the variant divided by the conversion observed in the reaction with SEQ ID NO: 1412 (where the conversion may be set as the average of replicates or else the highest single sample as appropriate). Conversion was calculated as 3′PO4 product (SEQ ID NO: 2133) peak areas over the total of the unreacted substrate, byproduct, and 3′PO4 product (SEQ ID NO: 2133) peak areas. The results are shown in Table 36.2.

TABLE 36.2
Relative to SEQ ID NO: 1412

| SEQ ID | | Activity FIOP |
|---|---|---|
| NO: | Amino Acid Differences | Conversion Relative |
| (nt/aa) | (Relative to SEQ ID NO: 1412) | to SEQ ID NO: 1412 |
| 1479/1480 | S125E | +++ |
| 1481/1482 | A61G | + |
| 1483/1484 | V83R | +++ |
| 1485/1486 | D49Q | +++ |
| 1497/1498 | A2V | + |
| 1501/1502 | S76D | +++ |
| 1513/1514 | A2W | + |
| 1529/1530 | D49G | ++ |
| 1531/1532 | D49N | + |
| 1623/1624 | N85R | + |
| 1637/1638 | N85M | ++ |
| 1709/1710 | N85A | ++ |
| 1717/1718 | A61E | +++ |
| 1719/1720 | Q195K | ++ |
| 1721/1722 | S19T | ++ |
| 1723/1724 | A35C | ++ |
| 1725/1726 | E53K | + |
| 1727/1728 | E78P | + |
| 1729/1730 | Q195A | + |
| 1731/1732 | E45Q | + |
| 1733/1734 | H194A | + |
| 1735/1736 | K170C | + |
| 1737/1738 | R48G | + |
| 1739/1740 | R48K | + |
| 1741/1742 | R171M | + |
| 1743/1744 | V83T | + |
| 1745/1746 | R171A | + |
| 1747/1748 | S121T | + |
| 1749/1750 | Q80G | + |
| 1751/1752 | K170I | + |
| 1753/1754 | D49S | + |
| 1755/1756 | K170T | + |
| 1757/1758 | S106R | + |
| 1759/1760 | N85W | + |
| 1761/1762 | A52K | + |
| 1763/1764 | A98G | + |
| 1765/1766 | D49R | + |
| 1767/1768 | K109A | + |
| 1769/1770 | R171Q | + |

Levels of increased activity were determined relative to the reference polypeptide of SEQ ID NO: 1412 and defined as follows: “+” 1.1 to 1.3, “++” 1.3 to 1.5, “+++” 1.5 to 1.9.

### Example 37

### Improvements Over SEQ ID NO: 1412 in Conversion to Tetraphosphorylated Nucleotide

### HTP Screening for Improved 3OK Variants

SEQ ID NO: 1412 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 37.1.

Reactions were performed at 2 μL scale in 96-well format 200 μL BioRad PCR plates. Reactions included substrate, LiKAcPO₄, MgCl₂, AcK0, 50% v/v lysate, in Tris-HC. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 37.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 37.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 55° C. and processed as described in Example 15.
The heat-treated and clarified HTP lysates were used in reactions.
Reaction conditions: Substrate - 1 mM fATP; Reaction buffer - 100 mM
Tris-HCl, pH 8, 50 mM LiKAcPO₄, 10 mM Magnesium chloride,
0.2 g/L AcK101; Lysate dilution - None; Reaction time - 16 hour
TdT coupling reaction conditions: Substrate - 10 μM SEQ ID NO: 2130,
10 μM above reaction mixture; Reaction buffer - 20 mM TEoA, pH 7.8,
50 mM CoCl₂, 0.001 U/μL IPP; Enzyme: 4 μM SEQ ID NO: 2127

Activity relative to SEQ ID NO: 1412 (Activity FIOP) was calculated as the fold-improvement of conversion of the variant divided by the conversion observed in the reaction with SEQ ID NO: 1412 (where the conversion may be set as the average of replicates or else the highest single sample as appropriate). Conversion was calculated as 3′PO4 product (SEQ ID NO: 2149) peak areas over the total of the unreacted substrate, byproduct, and 3′PO4 product (SEQ ID NO: 2149) peak areas. The results are shown in Table 37.2.

TABLE 37.2
Relative to SEQ ID NO: 1412

| SEQ ID | | Activity FIOP |
|---|---|---|
| NO: | Amino Acid Differences | Conversion Relative |
| (nt/aa) | (Relative to SEQ ID NO: 1412) | to SEQ ID NO: 1412 |
| 1507/1508 | R186V | +++ |
| 1517/1518 | K94A | + |
| 1541/1542 | Q80T | +++ |
| 1567/1568 | R60V | + |
| 1571/1572 | K82H | + |
| 1579/1580 | K109D | + |
| 1587/1588 | V92A | + |
| 1601/1602 | K109S | ++ |
| 1611/1612 | V83L | + |
| 1617/1618 | M40R | ++ |
| 1625/1626 | R48V | + |
| 1635/1636 | G57K | + |
| 1671/1672 | R48E | + |
| 1673/1674 | W100N | + |
| 1679/1680 | V92T | + |
| 1691/1692 | K94G | ++ |
| 1701/1702 | P28E | ++ |
| 1703/1704 | R60S | +++ |
| 1729/1730 | Q195A | ++ |
| 1733/1734 | H194A | + |
| 1737/1738 | R48G | + |
| 1739/1740 | R48K | + |
| 1763/1764 | A98G | + |
| 1771/1772 | D49L | +++ |
| 1773/1774 | M127T | +++ |
| 1775/1776 | L193V | ++ |
| 1777/1778 | R48A | ++ |
| 1779/1780 | K94R | ++ |
| 1781/1782 | G57A | ++ |
| 1783/1784 | L198S | ++ |
| 1785/1786 | R171L | + |
| 1787/1788 | R48C | + |
| 1789/1790 | R48T | + |
| 1791/1792 | I104V | + |
| 1793/1794 | K109M | + |
| 1795/1796 | R186S | + |
| 1797/1798 | V51I | + |

Levels of increased activity were determined relative to the reference polypeptide of SEQ ID NO: 1412 and defined as follows: “+” 1.1 to 1.2, “++” 1.2 to 1.3, “+++” 1.3 to 1.6.

### Example 38

### Improvements Over SEQ ID NO: 1464 in Conversion to Tetraphosphorylated Nucleotide

### HTP Screening for Improved 3OK Variants

SEQ ID NO: 1464 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 38.1.

Reactions were performed at 2 μL scale in 96-well format 200 μL BioRad PCR plates. Reactions included substrate, LiKAcPO₄, MgCl₂, AcK10m, 50% v/v lysate, in Tris-HC. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 38.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 38.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 65° C. and processed as described in Example 15.
The heat-treated and clarified HTP lysates were used in reactions.
Reaction conditions: Substrate - 10 mM ATP; Reaction buffer - 100 mM
Tris-HCl, pH 8, 50 mM LiKAcPO₄, 10 mM Magnesium chloride,
0.2 g/L AcK101; Lysate dilution - None; Reaction time - 16 hour
TdT coupling reaction conditions: Substrate - 10 μM SEQ ID NO: 2130,
10 μM above reaction mixture; Reaction buffer - 20 mM TEoA, pH 7.8,
50 mM CoCl₂, 0.001 U/μL IPP; Enzyme: 4 μM SEQ ID NO: 2127

Activity relative to SEQ ID NO: 1464 (Activity FIOP) was calculated as the fold-improvement of conversion of the variant divided by the conversion observed in the reaction with SEQ ID NO: 1464 (where the conversion may be set as the average of replicates or else the highest single sample as appropriate). Conversion was calculated as 3′PO4 product (SEQ ID NO: 2145) peak areas over the total of the unreacted substrate, byproduct, and 3′PO4 product (SEQ ID NO: 2145) peak areas. The results are shown in Table 38.2.

TABLE 38.2
Relative to SEQ ID NO: 1464

| SEQ ID | | Activity FIOP |
|---|---|---|
| NO: | Amino Acid Differences | Conversion Relative |
| (nt/aa) | (Relative to SEQ ID NO: 1464) | to SEQ ID NO: 1464 |
| 1799/1800 | E64R/F150L/N181T | +++ |
| 1801/1802 | E64R/A72D/I115V/F150L | +++ |
| 1803/1804 | G10S/L27F/A38S/D49M | ++ |
| 1805/1806 | L117G/F150L | ++ |
| 1807/1808 | L27F/R60A/V83R/S125E | ++ |
| 1809/1810 | E64R/H103M/F150L/N181T | ++ |
| 1811/1812 | H103F/I182E | ++ |
| 1813/1814 | A72D/H103M/S125A/F150L/R160E/ | ++ |
| | N181S | |
| 1815/1816 | G68S/A72L/V83R/V175E | ++ |
| 1817/1818 | K41A/E64R/A72D/H103M/R160K | ++ |
| 1819/1820 | F150L/R160E/N181V | + |
| 1821/1822 | V3T/A20C/R74F/H103F | + |
| 1823/1824 | L27F/V83R/L113A | + |
| 1825/1826 | I192L | + |
| 1827/1828 | H103L/I182P | + |
| 1829/1830 | F150L/N181S | + |
| 1831/1832 | T151G | + |
| 1833/1834 | R60A/V175E | + |
| 1835/1836 | E64R/S161V | + |
| 1837/1838 | K41A/E64R/H103M/L117G/F150L/ | + |
| | R160K/S161V | |
| 1839/1840 | R74F/S165I | + |
| 1841/1842 | A72D/H103M/Q124A/R160K/S161V | + |
| 1843/1844 | R60L/A61P | + |
| 1845/1846 | D49M/G68S/Y134M | + |
| 1847/1848 | V39C | + |
| 1849/1850 | L27F/D49Q/R74H | + |
| 1851/1852 | I182E | + |
| 1853/1854 | G10S/V83R | + |
| 1855/1856 | R23A/L27F/D49Q/V83R/S125E/ | + |
| | L141I | |
| 1857/1858 | A72D/Q124Y/F150L/R160S/N181V | + |
| 1859/1860 | R160K/N181T | + |
| 1861/1862 | E64R | + |
| 1863/1864 | A61P/T110A/A146H/T151G | + |
| 1865/1866 | G10S/R23A/L27F/A38S/D49Q/ | + |
| | L113A | |
| 1867/1868 | D49M/R60A | + |
| 1869/1870 | D49Q/E64A/M96T/L113A/V175E | + |

Levels of increased activity were determined relative to the reference polypeptide of SEQ ID NO: 1464 and defined as follows: “+” 1.19 to 2.7, “++” 2.7 to 5.3, “+++” 5.3 to 13.2.

### Example 39

### Improvements Over SEQ ID NO: 1464 in Conversion to Tetraphosphorylated Nucleotide

### HTP Screening for Improved 3OK Variants

SEQ ID NO: 1464 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 39.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 39.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 39.1
All lysis, reaction, quench, and analytical properties

Lysis conditions: Lysis buffer - 50 mM TEoA buffer, pH 7.5, 0.1 g/L
lysozyme; Lysis buffer volume - 400 μL; Lysate pre-treatment - Lysates
were heat-treated at 55° C. and processed as described in Example 15.
The heat-treated and clarified HTP lysates were used in reactions.
Reaction conditions: Substrate - 1 mM fATP; Reaction buffer - 100 mM
Tris-HCl, pH 8, 50 mM LiKAcPO₄, 10 mM Magnesium chloride,
0.2 g/L AcK101; Lysate dilution - None; Reaction time - 16 hour
TdT coupling reaction conditions: Substrate - 10 μM SEQ ID NO: 2130,
10 μM above reaction mixture; Reaction buffer - 20 mM TEoA, pH 7.8,
50 mM CoCl₂, 0.001 U/μL IPP; Enzyme: 4 μM SEQ ID NO: 2127

Activity relative to SEQ ID NO: 1464 (Activity FIOP) was calculated as the fold-improvement of conversion of the variant divided by the conversion observed in the reaction with SEQ ID NO: 1464 (where the conversion may be set as the average of replicates or else the highest single sample as appropriate). Conversion was calculated as 3′PO4 product (SEQ ID NO: 2149) peak areas over the total of the unreacted substrate, byproduct, and 3′PO4 product (SEQ ID NO: 2149) peak areas. The results are shown in Table 39.2.

TABLE 39.2
Relative to SEQ ID NO: 1464

| SEQ ID | | Activity FIOP |
|---|---|---|
| NO: | Amino Acid Differences | Conversion Relative |
| (nt/aa) | (Relative to SEQ ID NO: 1464) | to SEQ ID NO: 1464 |
| 1801/1802 | E64R/A72D/I115V/F150L | +++ |
| 1813/1814 | A72D/H103M/S125A/F150L/R160E/ | + |
| | N181S | |
| 1825/1826 | I192L | + |
| 1871/1872 | A61P/T110A/S165Q | +++ |
| 1873/1874 | A20C/H103L/I192L | ++ |
| 1875/1876 | A52S/A61P | + |
| 1877/1878 | A72D | + |

Levels of increased activity were determined relative to the reference polypeptide of SEQ ID NO: 1464 and defined as follows: “+” 1.1 to 1.2, “++” 1.2 to 1.3, “+++” 1.3 to 1.33.

### Example 40

### Improvements Over SEQ ID NO: 1464 in Conversion to Tetraphosphorylated Nucleotide

### HTP Screening for Improved 3OK Variants

SEQ ID NO: 1464 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 40.1.

Reactions were performed at 2 μL scale in 96-well format 200 μL BioRad PCR plates. Reactions included substrate, LiKAcPO₄, MgCl₂, AcK10m, 50% v/v lysate, in Tris-HC. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 40.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 40.1
All lysis, reaction, quench, and analytical properties

Lysis conditions:
Lysis buffer - 50 mM TEoA buffer, pH 7.5, 0.1 g/L lysozyme;
Lysis buffer volume - 400 μL;
Lysate pre-treatment - Lysates were heat-treated at 55° C. and processed
as described in Example 15. The heat-treated and clarified HTP lysates
were used in reactions.
Reaction conditions:
Substrate - 10 mM CTP;
Reaction buffer - 100 mM Tris-HCl, pH 8, 50 mM LiKAcPO_{4,} 10 mM
Magnesium chloride, 0.2 g/L AcK101;
Lysate dilution - None;
Reaction time - 16 hour
TdT coupling reaction conditions:
Substrate - 10 μM SEQ ID NO: 2130, 10 μM above reaction mixture;
Reaction buffer - 20 mM TEoA, pH 7.8, 50 mM CoCl₂, 0.001 U/μL IPP;
Enzyme: 4 μM SEQ ID NO: 2127

Activity relative to SEQ ID NO: 1464 (Activity FIOP) was calculated as the fold-improvement of conversion of the variant divided by the conversion observed in the reaction with SEQ ID NO: 1464 (where the conversion may be set as the average of replicates or else the highest single sample as appropriate). Conversion was calculated as 3′PO4 product (SEQ ID NO: 2147) peak areas over the total of the unreacted substrate, byproduct, and 3′PO4 product (SEQ ID NO: 2147) peak areas. The results are shown in Table 40.2.

TABLE 40.2
Relative to SEQ ID NO: 1464

| SEQ ID | | Activity FIOP |
|---|---|---|
| NO: | Amino Acid Differences | Conversion Relative |
| (nt/aa) | (Relative to SEQ ID NO: 1464) | to SEQ ID NO: 1464 |
| 1879/1880 | G68E | +++ |
| 1881/1882 | N148G | +++ |
| 1883/1884 | F150L | +++ |
| 1885/1886 | I182R/G205D | ++ |
| 1887/1888 | F75L | ++ |
| 1889/1890 | S16T | ++ |
| 1891/1892 | T151P | ++ |
| 1893/1894 | S165Q | ++ |
| 1895/1896 | A20R | ++ |
| 1897/1898 | L93V | ++ |
| 1899/1900 | G122E | + |
| 1901/1902 | I182R | + |
| 1903/1904 | V139T | + |
| 1905/1906 | A20H | + |
| 1907/1908 | N85T | + |
| 1909/1910 | V88L | + |
| 1911/1912 | G89L | + |
| 1913/1914 | Y134W | + |
| 1915/1916 | A35L | + |
| 1917/1918 | A146S | + |
| 1919/1920 | N85L | + |
| 1921/1922 | S161L | + |
| 1923/1924 | M127V | + |
| 1879/1880 | G68E | +++ |
| 1881/1882 | N148G | +++ |
| 1883/1884 | F150L | +++ |
| 1885/1886 | I182R/G205D | ++ |
| 1887/1888 | F75L | ++ |
| 1889/1890 | S16T | ++ |
| 1891/1892 | T151P | ++ |
| 1893/1894 | S165Q | ++ |
| 1895/1896 | A20R | ++ |
| 1897/1898 | L93V | ++ |
| 1899/1900 | G122E | + |
| 1901/1902 | I182R | + |
| 1903/1904 | V139T | + |
| 1905/1906 | A20H | + |
| 1907/1908 | N85T | + |
| 1909/1910 | V88L | + |
| 1911/1912 | G89L | + |
| 1913/1914 | Y134W | + |
| 1915/1916 | A35L | + |
| 1917/1918 | A146S | + |
| 1919/1920 | N85L | + |
| 1921/1922 | S161L | + |
| 1923/1924 | M127V | + |

Levels of increased activity were determined relative to the reference polypeptide of SEQ ID NO: 1464 and defined as follows: “+” 1.1 to 1.3, “++” 1.3to 1.4, “+++” 1.4 to 1.7.

### Example 41

### Improvements Over SEQ ID NO: 1464 in Conversion to Tetraphosphorylated Nucleotide

### HTP Screening for Improved 3OK Variants

SEQ ID NO: 1464 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 41.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 41.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 41.1
All lysis, reaction, quench, and analytical properties

Lysis conditions:
Lysis buffer - 50 mM TEoA buffer, pH 7.5, 0.1 g/L lysozyme;
Lysis buffer volume - 400 μL;
Lysate pre-treatment - Lysates were heat-treated at 65° C. and processed
as described in Example 15. The heat-treated and clarified HTP lysates
were used in reactions.
Reaction conditions:
Substrate - 10 mM ATP;
Reaction buffer - 100 mM Tris-HCl, pH 8, 50 mM LiKAcPO_{4,} 10 mM
Magnesium chloride, 0.2 g/L AcK101;
Lysate dilution - None;
Reaction time - 16 hour
TdT coupling reaction conditions:
Substrate - 10 μM SEQ ID NO: 2130, 10 μM above reaction mixture;
Reaction buffer - 20 mM TEoA, pH 7.8, 50 mM CoCl₂, 0.001 U/μL IPP;
Enzyme: 4 μM SEQ ID NO: 2127

Activity relative to SEQ ID NO: 1464 (Activity FIOP) was calculated as the fold-improvement of conversion of the variant divided by the conversion observed in the reaction with SEQ ID NO: 1464 (where the conversion may be set as the average of replicates or else the highest single sample as appropriate). Conversion was calculated as 3′PO4 product (SEQ ID NO: 2145) peak areas over the total of the unreacted substrate, byproduct, and 3′PO4 product (SEQ ID NO: 2145) peak areas. The results are shown in Table 41.2.

TABLE 41.2
Relative to SEQ ID NO: 1464

| SEQ ID | | Activity FIOP |
|---|---|---|
| NO: | Amino Acid Differences | Conversion Relative |
| (nt/aa) | (Relative to SEQ ID NO: 1464) | to SEQ ID NO: 1464 |
| 1879/1880 | G68E | +++ |
| 1881/1882 | N148G | + |
| 1883/1884 | F150L | +++ |
| 1885/1886 | I182R/G205D | + |
| 1887/1888 | F75L | ++ |
| 1901/1902 | I182R | + |
| 1903/1904 | V139T | +++ |
| 1905/1906 | A20H | +++ |
| 1909/1910 | V88L | ++ |
| 1911/1912 | G89L | + |
| 1915/1916 | A35L | + |
| 1919/1920 | N85L | + |
| 1921/1922 | S161L | + |
| 1925/1926 | S121G | +++ |
| 1927/1928 | A20G | +++ |
| 1929/1930 | M22L | +++ |
| 1931/1932 | R160H | +++ |
| 1933/1934 | I176L | +++ |
| 1935/1936 | A146D | ++ |
| 1937/1938 | R142G | ++ |
| 1939/1940 | D136S | ++ |
| 1941/1942 | R81N | ++ |
| 1943/1944 | L141V | ++ |
| 1945/1946 | G68S | + |
| 1947/1948 | S121T | + |
| 1949/1950 | A35V | + |
| 1951/1952 | D137S | + |
| 1953/1954 | R71A | + |
| 1955/1956 | F150H | + |
| 1957/1958 | T151A | + |
| 1959/1960 | V18L | + |
| 1961/1962 | L7V | + |
| 1963/1964 | A146N | + |
| 1965/1966 | F75N | + |
| 1967/1968 | F150C | + |
| 1969/1970 | R67K | + |
| 1971/1972 | E153A | + |
| 1973/1974 | I182P | + |
| 1975/1976 | T185A | + |
| 1977/1978 | G68Y | + |
| 1979/1980 | R160T | + |
| 1981/1982 | R160S | + |
| 1983/1984 | R160V | + |

Levels of increased activity were determined relative to the reference polypeptide of SEQ ID NO: 1464 and defined as follows: “+” 1.2 to 4.1, “++” 4.1 to 7.1, “+++” 7.1 to 11.9.

### Example 42

### Improvements Over SEQ ID NO: 1464 in Conversion to Tetraphosphorylated Nucleotide

### HTP Screening for Improved 3OK Variants

SEQ ID NO: 1464 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 42.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 42.1. The reaction plates were heat-sealed with a peelable aluminum seal and incubated at 30° C. for 1 h and 95° C. for 2 min then held at 4° C. until the reaction was quenched.

Reactions were quenched and analyzed by CE analysis as described in Example 14.

TABLE 42.1
All lysis, reaction, quench, and analytical properties

Lysis conditions:
Lysis buffer - 50 mM TEoA buffer, pH 7.5, 0.1 g/L lysozyme;
Lysis buffer volume - 400 μL;
Lysate pre-treatment - Lysates were heat-treated at 55° C. and processed
as described in Example 15. The heat-treated and clarified HTP lysates
were used in reactions.
Reaction conditions:
Substrate - 1 mM fATP;
Reaction buffer - 100 mM Tris-HCl, pH 8, 50 mM LiKAcPO_{4,} 10 mM
Magnesium chloride, 0.2 g/L AcK101;
Lysate dilution - None;
Reaction time - 16 hour
TdT coupling reaction conditions:
Substrate - 10 μM SEQ ID NO: 2130, 10 μM above reaction mixture;
Reaction buffer - 20 mM TEoA, pH 7.8, 50 mM CoCl₂, 0.001 U/μL IPP;
Enzyme: 4 μM SEQ ID NO: 2127

Activity relative to SEQ ID NO: 1464 (Activity FIOP) was calculated as the fold-improvement of conversion of the variant divided by the conversion observed in the reaction with SEQ ID NO: 1464 (where the conversion may be set as the average of replicates or else the highest single sample as appropriate). Conversion was calculated as 3′PO4 product (SEQ ID NO: 2149) peak areas over the total of the unreacted substrate, byproduct, and 3′P04 product (SEQ ID NO: 2149) peak areas. The results are shown in Table 42.2.

TABLE 42.2
Relative to SEQ ID NO: 1464

| SEQ ID | | Activity FIOP |
|---|---|---|
| NO: | Amino Acid Differences | Conversion Relative |
| (nt/aa) | (Relative to SEQ ID NO: 1464) | to SEQ ID NO: 1464 |
| 1893/1894 | S165Q | ++ |
| 1897/1898 | L93V | + |
| 1915/1916 | A35L | ++ |
| 1921/1922 | S161L | +++ |
| 1923/1924 | M127V | + |
| 1949/1950 | A35V | + |
| 1953/1954 | R71A | + |
| 1975/1976 | T185A | + |
| 1985/1986 | A35M | +++ |
| 1987/1988 | M127A | +++ |
| 1989/1990 | L113W | +++ |
| 1991/1992 | I30V | +++ |
| 1993/1994 | K95V | +++ |
| 1995/1996 | M40R | ++ |
| 1997/1998 | G89M | ++ |
| 1999/2000 | A35R | ++ |
| 2001/2002 | A35D | ++ |
| 2003/2004 | A20S | ++ |
| 2005/2006 | A20I | + |
| 2007/2008 | A20K | + |
| 2009/2010 | A36S | + |
| 2011/2012 | N85E | + |
| 2013/2014 | I30G | + |
| 2015/2016 | R23A | + |
| 2017/2018 | A20Q | + |
| 2019/2020 | G89T | + |
| 2021/2022 | V18A | + |
| 2023/2024 | V29I | + |
| 2025/2026 | R142S | + |
| 2027/2028 | R37V | + |
| 2029/2030 | M40L | + |
| 2031/2032 | A146Q | + |
| 2033/2034 | R142A | + |
| 2035/2036 | A38I | + |

Levels of increased activity were determined relative to the reference polypeptide of SEQ ID NO: 1464 and defined as follows: “+” 1.1 to 1.3, “++” 1.3 to 1.5, “+++” 1.5 to 1.8.

### Example 43

### Improvements Over SEQ ID NO: 1464 in Conversion to Tetraphosphorylated Nucleotide

### HTP Screening for Improved 3OK Variants

SEQ ID NO: 1464 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 43.1.

Reactions were performed at 2 μL scale in 96-well format 200 μL BioRad PCR plates. Reactions included substrate, LiKAcPO₄, MgCl₂, AcK101, 50% v/v lysate, in Tris-HC. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 43.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 43.1
All lysis, reaction, quench, and analytical properties

Lysis conditions:
Lysis buffer - 50 mM TEoA buffer, pH 7.5, 0.1 g/L lysozyme;
Lysis buffer volume - 400 μL;
Lysate pre-treatment - Lysates were heat-treated at 55° C. and processed
as described in Example 15. The heat-treated and clarified HTP lysates
were used in reactions.
Reaction conditions:
Substrate - 1 mM GTP-αS;
Reaction buffer - 100 mM Tris-HCl, pH 8, 50 mM LiKAcPO_{4,} 10 mM
Magnesium chloride, 0.2 g/L AcK101;
Lysate dilution - None;
Reaction time - 16 hour
TdT coupling reaction conditions:
Substrate - 10 μM SEQ ID NO: 2130, 10 μM above reaction mixture;
Reaction buffer - 20 mM TEoA, pH 7.8, 50 mM CoCl₂, 0.001 U/μL IPP;
Enzyme: 4 μM SEQ ID NO: 2127

Activity relative to SEQ ID NO: 2054 (Activity FIOP) (SEQ ID 1464 has no activity, SEQ ID 2054 was used instead for FIOP calculation.) was calculated as the fold-improvement of conversion of the variant divided by the conversion observed in the reaction with SEQ ID 2054 (where the conversion may be set as the average of replicates or else the highest single sample as appropriate). Conversion was calculated as 3′PO4 product (SEQ ID NO: 2158) peak areas over the total of the unreacted substrate, byproduct, and 3′PO4 product (SEQ ID NO: 2158) peak areas. The results are shown in Table 43.2.

TABLE 43.2
Relative to SEQ ID NO: 2054

| SEQ ID | | Activity FIOP |
|---|---|---|
| NO: | Amino Acid Differences | Conversion Relative |
| (nt/aa) | (Relative to SEQ ID NO: 1464) | to SEQ ID NO: 2054 |
| 2037/2038 | V133R | +++ |
| 2039/2040 | T8R | ++ |
| 2041/2042 | V88W | ++ |
| 2043/2044 | A155P | ++ |
| 2045/2046 | L113R | ++ |
| 2047/2048 | S161V | + |
| 2049/2050 | I11P | + |
| 2051/2052 | R143V | + |
| 2053/2054 | K15Y | + |

Levels of increased activity were determined relative to the reference polypeptide of SEQ ID NO: 2054 and defined as follows: “+” 1.0 to 2.0, “++” 2.0 to 2.7, “+++” 2.7 to 2.9.

### Example 44

### Improvements Over SEQ ID NO: 1800 in Conversion to Tetraphosphorylated Nucleotide

### HTP Screening for Improved 3OK Variants

SEQ ID NO: 1800 was selected as the parent 3OK enzyme. Libraries of engineered genes were produced from the parent gene using well-established techniques (e.g., saturation mutagenesis and recombination of previously identified beneficial mutations). The polypeptides encoded by each gene were produced in HTP and prepared as described in Table 44.1.

Reactions were performed at 2 μL scale in 96-well format 200 μL BioRad PCR plates. Reactions included substrate, LiKAcPO₄, MgCl₂, AcK101, 50% v/v lysate, in Tris-HCl. The reactions were set up as follows: (i) all reaction components except for lysate were pre-mixed in a single solution, and 1 μL of this solution was aliquoted into each well of the 96-well plate; (ii) 1 μL lysate was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 44.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 44.1
All lysis, reaction, quench, and analytical properties

Lysis conditions:
Lysis buffer - 50 mM TEoA buffer, pH 7.5, 0.1 g/L lysozyme;
Lysis buffer volume - 400 μL;
Lysate pre-treatment - Lysates were heat-treated at 66° C. and processed
as described in Example 15. The heat-treated and clarified HTP lysates
were used in reactions.
Reaction conditions:
Substrate - 10 mM ATP;
Reaction buffer - 100 mM Tris-HCl, pH 8, 50 mM LiKAcPO_{4,} 10 mM
Magnesium chloride, 0.2 g/L AcK101;
Lysate dilution - None;
Reaction time - 16 hour
TdT coupling reaction conditions: Substrate - 10 μM SEQ ID NO: 2130,
10 μM above reaction mixture;
Reaction buffer - 20 mM TEoA, pH 7.8, 50 mM CoCl₂, 0.001 U/μL IPP;
Enzyme: 4 μM SEQ ID NO: 2127

Activity relative to SEQ ID 1800 (Activity FIOP) was calculated as the fold-improvement of conversion of the variant divided by the conversion observed in the reaction with SEQ ID 1800 (where the conversion may be set as the average of replicates or else the highest single sample as appropriate). Conversion was calculated as 3′PO4 product (SEQ ID NO: 2145) peak areas over the total of the unreacted substrate, byproduct, and 3′PO4 product (SEQ ID NO: 2145) peak areas. The results are shown in Table 44.2.

TABLE 44.2
Relative to SEQ ID NO: 1800

| | | Activity |
|---|---|---|
| | | FIOP |
| | | Conversion |
| SEQ ID | | Relative to |
| NO: | Amino Acid Differences | SEQ ID NO: |
| (nt/aa) | (Relative to SEQ ID NO: 1800) | 1800 |
| 2055/2056 | R186V | +++ |
| 2057/2058 | S165Q | +++ |
| 2059/2060 | R60V/L193V | +++ |
| 2061/2062 | P56L/S76D/Q80T/K170T | ++ |
| 2063/2064 | E53K/P56L/R60V/S76G | ++ |
| 2065/2066 | S76G/Q80T | ++ |
| 2067/2068 | P56L/R60V/N85M/L193V | ++ |
| 2069/2070 | R48E/E53K/R60V/S76D/Q80T/L193V | ++ |
| 2071/2072 | P56L/R60V | ++ |
| 2073/2074 | M40R/V92A/I104V | ++ |
| 2075/2076 | P56L/R60V/S76D/E78V/Q80T | + |
| 2077/2078 | R60A/A61E | + |
| 2079/2080 | E101L/K109S/L198S | + |
| 2081/2082 | L27F | + |
| 2083/2084 | L27F/D49G/V51I | + |
| 2085/2086 | P56L/N85M/I104V | + |
| 2087/2088 | R48A | + |
| 2089/2090 | M40R | + |
| 2091/2092 | D49Q | + |
| 2093/2094 | P56L/E167A/L193V | + |
| 2095/2096 | R48E/P56L/R60V/S76D/E167A/K170T/L193V | + |
| 2097/2098 | P56L/S76G/Q80T/L193V | + |
| 2099/2100 | E101L | + |
| 2101/2102 | S125E | + |
| 2103/2104 | L27F/D49L/R171A | + |
| 2105/2106 | D49N | + |
| 2107/2108 | R60V | + |
| 2109/2110 | A98G | + |
| 2111/2112 | R171L/R186P | + |

“Levels of increased activity were determined relative to the reference polypeptide of SEQ ID NO: 1800 and defined as follows: ““+”” 1.1 to 2.9, ““++”” 2.9 to 5.0, ““+++”” 5.0 to 9.8.

### Example 45

### Improvements Over SEQ ID NO: 2078 in Conversion to Tetraphosphorylated Nucleotide

### Characterization of Shake Flask Purified Proteins for Improved 3OK Variants

SEQ ID NO: 2078 was selected as the parent 3OK enzyme. Six shake-flask variants including the parent SEQ ID NO: 2078 were grown, expressed, and purified as described in Example 7 method 2

Reactions were performed at 2 μL scale in 96-well format 200 μL BioRad PCR plates. Reactions included substrate, LiKAcPO₄, MgCl₂, AcK101, 25% 3OK solution, in Tris-HCl. The reactions were set up as follows: (i) all reaction components except for 3OK solution were pre-mixed in a single solution, and 1.5 μL of this solution was aliquoted into each well of the 96-well plate; (ii) 0.5 μL 3OK solution was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 45.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 45.1
Reaction, quench, and analytical properties

Reaction conditions:
Substrate - 1 mM fATP;
Reaction buffer - 100 mM Tris-HCl, pH 8, 50 mM LiKAcPO_{4,}
10 mM Magnesium chloride, 0.2 g/L AcK101;
3OK - 39 uM;
Reaction time - 16 hour
TdT coupling reaction conditions:
Substrate - 10 μM SEQ ID NO: 2130, 10 μM above
reaction mixture;
Reaction buffer - 20 mM TEoA, pH 7.8, 50 mM
CoCl₂, 0.001 U/μL IPP;
Enzyme: 4 μM SEQ ID NO: 2127
Quench conditions:
Reactions were quenched, and sample preparation as
described in Example 14

Activity relative to SEQ ID 2078 (Activity FIOP) was calculated as the fold-improvement of conversion of the variant divided by the conversion observed in the reaction with SEQ ID 2078 (where the conversion may be set as the average of replicates or else the highest single sample as appropriate).

Conversion was calculated as 3′PO4 product (SEQ ID NO: 2149) peak areas over the total of the unreacted substrate, byproduct, and 3′PO4 product (SEQ ID NO: 2149) peak areas. The results are shown in Table 45.2.

TABLE 45.2
Relative to SEQ ID NO: 2078

| SEQ ID | | Activity FIOP |
|---|---|---|
| NO: | Amino Acid Differences | Conversion Relative |
| (nt/aa) | (Relative to SEQ ID NO: 2078) | to SEQ ID NO: 2078 |
| 2113/2114 | S165Q | +++ |
| 2115/2116 | W100N | ++ |
| 2117/2118 | L193V | + |
| 2119/2120 | R48E | + |
| 2121/2122 | A52D | + |

Levels of increased activity were determined relative to the reference polypeptide of SEQ ID NO: 2078 and defined as follows: “+” 1.1 to 1.3), “++” 1.3 to 15, “+++” 1.5 to 1.7.

### Example 46

### Evaluation of Deoxynucleotide Triphosphate as Substrates as Determined by Conversion to the 3′PO4 Product

Activity of SEQ ID NO: 2078 with Deoxynucleotide Triphosphate

SEQ ID NO: 2078 was grown, expressed, and purified as described in Example 7 using method 2.

Reactions were performed at 2 μL scale in 96-well format 200 μL BioRad PCR plates. Reactions included substrate, LiKAcPO₄, MgCl₂, AcK101, 25% v/v 3OK solution, in Tris-HCl. The reactions were set up as follows: (i) all reaction components except for 3OK solution were pre-mixed in a single solution, and 1.5 μL of this solution was aliquoted into each well of the 96-well plate; (ii) 0.5 μL 3OK solution was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled oligo described in Table 46.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 46.1
All lysis, reaction, quench, and analytical properties

Reaction conditions:
Substrate - 1 mM dATP; 1 mM dGTP; 1 mM dCTP; 1
mM dTTP;
Reaction buffer - 100 mM Tris-HCl, pH 8, 50 mM LiKAcPO_{4,}
10 mM Magnesium chloride, 0.2 g/L AcK101;
3OK solution- 39 μM;
Reaction time - 16 hour
TdT coupling reaction conditions:
Substrate - 10 uM SEQ ID NO: 2130, 10 μM above
reaction mixture;
Reaction buffer - 20 mM TEoA, pH 7.8, 50 mM CoCl₂,
0.001 U/μL IPP;
Enzyme: 4 μM SEQ ID NO: 2127

Activity of SEQ ID 2078 (% Conversion) was calculated as 3′PO₄ product (SEQ ID NO: 2159, 2160, 2161, 2162) peak areas over the total of the unreacted substrate, byproduct, and 3′PO₄ product (SEQ ID NO: 2159, 2160, 2161, 2162) peak areas. The results are shown in Table 46.2.

TABLE 46.2
Percent conversion of deoxynucleotide triphosphates
to 3′ phosphorylated product by SEQ ID NO: 2078

| Product SEQ ID NO: | % Conversion |
|---|---|
| 2159 | +++ |
| 2160 | ++ |
| 2161 | ++ |
| 2162 | + |

Percent conversion in reactions with SEQ ID NO: 2078 are defined as follows:
“+” 0.5-1.0,
“++”1.0 to 4.0,
“+++” 4.0-6.0.

### Example 47

### Conversion of 2′Deoxyfluoro Adenosine (fA) to 3′Phosphorylated-fATP in a One-Pot Two-Step Biocatalytic Cascade Using Evolved Enzymes

Biocatalytic Conversion of fA to fATP

The enzymes SEQ ID NO: 2164 (nucleoside kinase variant), SEQ ID NO: 2166 (adenylate kinase variant), SEQ ID NO: 2124 (acetate kinase variant), and 3OK (SEQ ID NO: 2114) were expressed and purified as described in Example 7, method 2.

Biocatalytic Conversion of fA to fATP

To convert 2′deoxyfluoro adenosine (fA) to 2′deoxyfluro-adenosine triphosphate (fATP), each enzyme SEQ ID NO: 2164, SEQ ID NO: 2166, and SEQ ID NO: 2124 was added to a 1 mL reaction at a final concentration of 1 μM. The reaction contained 50 mM Tris (pH 8.0), 50 mM lithium potassium acetylphosphate, 10 μM ATP, 10 mM MgCl₂, and 10 mM nucleoside. The reaction was incubated in an Eppendorf Thermomixer at 30° C. and 400 rpm for 120 minutes. A 5 μL aliquot was then quenched and diluted 40-fold with 75% methanol and analyzed by HPLC using a Zorbax RR StableBond Aq, 3.0×150 mm, 3.5 μm (Agilent, #863954-314) column as a stationary phase and a mobile phase consisting of 50 mM potassium phosphate (pH 7) with 2 mM tetrabutylammonium hydrogensulfate (Solvent A), acetonitrile (solvent B), and water (Solvent C). Products were detected by UV absorption at 254 n. Based on this analysis, the quenched reaction contained 97% of fATP.

Purification of Crude fATP

To purify the crude fATP, 0.5 mL was removed from the crude reaction and placed in an Amicon Ultra—0.5 mL centrifugal filter which was then centrifuged at 14,000 rpm for 10 min. The filtrate was collected and purified by anion exchange chromatography using a pre-packed 5.0 mL Bio-Rad EconoFit Macro-Prep High Q anion exchange column. A 1.0 mL/min flow rate was used with a 0.25 to 0.4 M NH4HCO₃ buffer gradient over 90 mL (18 column volumes), collecting 3 mL fractions. The fractions were analyzed by UV-Vis absorbance at 260 nm to identify which contained nucleotide, and from each a 50 μL aliquot was transferred to a 96 well round bottom plate and dried overnight at room temperature on a vacufuge. These were reconstituted with 150 μL of water and analyzed by the same HPLC method above to identify the purest fractions, which were subsequently lyophilized and then reconstituted into 450 μL milli-Q water providing purified fATP in 6.1 mM concentration.

Biocatalytic Conversion of fATP to 3′Phosphorylated-fATP

To convert fATP to 3′phosphorylated-fATP, reactions were performed at 50 μL scale in a Costar round-bottom 96-well plate. Reactions contained 100 mM Tris (pH 8.0), 50 mM lithium potassium acetylphosphate, 10 mM magnesium chloride, 0.2 g/L SEQ ID NO: 2124, 40 μM SEQ ID NO: 2114, and 1 mM fATP from the preceding reaction, including two conditions both the purified material and as the crude reaction mixture. Following setup, reactions were then heat-sealed and mixed by briefly vortexing, then incubated in a Multitron Infors shaker at 30° C. and 400 rpm for 16 hours. Reactions were then quenched with the addition of 150 μL of methanol, sealed, and mixed by vortexing prior to HPLC analysis (as described above).

Activity of SEQ ID 2114 (% Conversion) was calculated as 3′PO₄ product peak areas over the total of the unreacted substrate, byproduct, and 3′PO₄ product and peak areas. The results are shown in Table 47.1.

TABLE 47.1
Conversion of fATP to 3′phosphorylated-
fATP by SEQ ID NO: 2114

| | Percent Conversion to |
|---|---|
| Source of fATP | 3′phosphorylated-fATP |
| Unpurified biocatalytic reaction mixture | + |
| Purified fATP | + |

Percent conversion in reactions with SEQ ID NO: 2114 are defined as follows: “+” 3.8-5.5.

### Example 48

### Ribonucleotide and Modified Nucleotide Activity Improvement by Evolved 3OKinases

SEQ ID NO: 10, SEQ ID NO: 372, SEQ ID NO: 496, SEQ ID NO: 1464, SEQ ID NO: 1800, SEQ ID NO: 2078, SEQ ID NO: 2114 were selected as the parents of 3OK enzymes. Seven shake-flask parent variants were grown, expressed, and purified as described in Example 7, method 2.

Reactions were performed at 2 μL scale in 96-well format 200 μL BioRad PCR plates. Reactions included substrate, LiKAcPO₄, MgCl₂, AcK101, 25% 3OK solution, in Tris-HCl. The reactions were set up as follows: (i) all reaction components except for 3OK solution were pre-mixed in a single solution, and 1.5 μL of this solution was aliquoted into each well of the 96-well plate; (ii) 0.5 μL 3OK solution was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. The reaction mixture was further diluted to 40 μM by water, then coupled with FAM labeled TdT oligo as described in Table 48.1. The reaction plates were heat-sealed with a peelable aluminum seal and incubated at 30° C. for 1 h and 95° C. for 2 min, then held at 4° C. until the reaction was quenched.

Reactions were quenched and analyzed by CE analysis as described in Example 14.

TABLE 48.1
Reaction, quench, and analytical properties

Reaction conditions:
Substrate - 10 mM ATP, 10 mM GTP, 10 mM CTP, 10 mM UTP, 1 mM
fATP, 1 mM GTP-αS;
Reaction buffer - 100 mM Tris-HCl, pH 8, 50 mM LiKAcPO_{4,} 10 mM
Magnesium chloride, 0.2 g/L AcK101;
3OK - 39 uM;
Reaction time - 16 hour
TdT coupling reaction conditions:
Substrate - 10 μM SEQ ID: 2130, 10 μM above reaction mixture;
Reaction buffer - 20 mM TEoA, pH 7.8, 50 mM CoCl₂, 0.001 U/μL IPP;
TdT enzyme: 4 μM SEQ ID: 2128
Quench conditions: Reactions were quenched, and sample preparation as
described in Example 14

Ribo and modified activities of SEQ ID: 9/10, SEQ ID: 371/372, SEQ ID: 495/496, SEQ ID: 1463/1464, SEQ ID: 1799/1800, SEQ ID: 2077/2078, SEQ ID: 2113/2114: Activity (% conversion) were calculated as 3′PO₄ product peak areas (SEQ ID: 2145, SEQ ID: 2146, SEQ ID: 2147, SEQ ID: 2148, SEQ ID: 2149, SEQ ID: 2158 over the total of the unreacted substrate (SEQ ID: 2130, byproduct, and 3′PO₄ product peak areas. The results are shown in Table 48.2.

TABLE 48.2
conversion of nucleotide triphosphates
to 3′phosphorylated products

| | Relative Activities of 3OKinase | | | | | |
|---|---|---|---|---|---|---|
| SEQ ID | variants on nucleotide substrates | | | | | |
| No: (nt/aa) | CTP | UTP | GTP | ATP | GTP-αS | fATP |
| 9/10 | − | − | − | + | − | − |
| 371/372 | − | − | + | +++ | − | − |
| 495/496 | + | + | ++ | +++ | + | − |
| 1463/1464 | ++ | ++ | +++ | +++ | + | + |
| 1799/1800 | ++ | ++ | +++ | +++ | +++ | + |
| 2077/2078 | +++ | +++ | +++ | ++++ | +++ | + |
| 2113/2114 | +++ | +++ | +++ | ++++ | +++ | ++ |

Levels of relative activity were measured for the listed variants and defined as follows: “−” 0.00 to 0.99, “+” ≥1.0, “++” >10.0, “+++” >30.0, “++++” >70.0.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes in the United States of America, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered 3′O-kinase comprising a polypeptide sequence having at least 85%, 90%, 91%, 92%, 93%, 94%, 95%, 96%, 97%, 98%, 99%, or more sequence identity to a reference sequence selected from SEQ ID NO: 10, 142, 372, 450, 496, 1042, 1180, 1412 1464, 1800, and 2078, or a functional fragment thereof, and one or more amino acid residue differences relative to the reference sequence.

2. The engineered 3′O-kinase of claim 1, wherein the polypeptide sequence comprises one or more amino acid residue differences as compared to the reference sequence of SEQ ID NO: 10 at amino acid positions selected from: 165, 89, 40, 13, 41, 74, 76, 93, 124, 150, 17, 32, 35, 36, 38, 39, 72, 60, 92, 116, 123, 138, 144, 148, 148, 156, 163, 177, 178, 179, and a combination thereof.

3. The engineered 3′O-kinase of claim 1, wherein the polypeptide sequence comprises one or more amino acid residue differences as compared to the reference sequence of SEQ ID NO: 10 at one or more amino acid positions selected from: 76/89/93/165, 13/76, 13/89, 13/124, 74/89, 76/93, 89/93, 89/124, 13/76/89/93, 13/76/89/93/124, 13/76/93, 13/76/93/124, 13/76/124, 13/89/124, 13/89/165, 74/89/93, 74/89/124, 76/89/93/124, 76/89/124/165, 76/89/165, 89/93/124, 13/36/38/39/40/74/89, 13/36/38/39/40/89, 13/36/38/39/72/76/89/93/124, 13/36/38/40, 13/36/38/40/72/74, 13/36/38/40/72/74/89/93, 13/36/38/40/72/74/93/156, 13/36/38/40/72/74/124, 13/36/38/40/72/76/89, 13/36/38/40/74/76, 13/36/38/40/76/89/93, 13/36/38/40/89, 13/36/39/40/72/76/89/124, 13/36/40/72/74/76/93, 13/36/40/72/156, 13/36/40/93/124, 13/38/39/40/72/76/93/165, 13/38/39/40/89/124/156, 13/38/40/89, 13/38/72/156, 13/40, 13/40/72/76/89, 13/72, 13/72/74/76/89/93, 13/72/74/76/89/93/124, 13/72/74/89/93, 13/72/74/89/93/124, 13/72/76, 13/72/76/89/93, 13/72/76/89/124, 13/72/76/124/156, 13/72/89, 13/72/89/93/124/156, 13/72/89/124, 13/72/89/124/165, 13/72/93/124, 13/72/124, 13/74/89/93, 13/74/89/93/124, 13/74/89/156, 13/76/89/93/156/165, 13/76/89/124/156, 13/76/89/156/165, 13/156, 36/38/39/40/72/74/76/124, 36/38/39/40/72/74/89, 36/38/39/40/74/76/89/93/124, 36/38/40/72/74/89/93/124/156, 36/39/40/72/76/89/93, 36/39/40/76/93/156, 36/40/72/74/89/93, 38/39, 38/39/40/72/74/76/89, 38/39/76, 38/40/72, 38/40/76/89/124, 38/40/89/124, 38/40/93, 38/40/156, 38/72/76/89, 38/72/89/93/124, 38/76/156, 39/40/72/76, 72/74/76/89/124, 72/74/76/124, 72/74/89, 72/74/89/93, 72/74/89/93/124, 72/74/93, 72/74/93/124, 72/76, 72/76/89/93, 72/76/124, 72/89/165, 72/93/124, 74/76/89, 74/76/89/93/124, 74/76/89/124, 74/89/93/156, 124/156, 138/139, and/or any combinations thereof.

4. The engineered 3′O-kinase of claim 1, wherein the polypeptide sequence comprises one or more amino residue difference as compared to the reference sequence of SEQ ID NO: 142 at one or more amino acid positions selected from: 13/76/93/198, 13/76/93, 13/76/198, 68, 68/103/181/182, 76, 82, 82/198, 83, 86, 88, 91, 93, 93/198, 103, 111, 169, 181, 182, 191,200, 210, and 211.

5. The engineered 3′O-kinase of claim 1, wherein the polypeptide sequence comprises one or more amino residue difference as compared to the reference sequence of SEQ ID NO: 372 at one or more amino acid positions selected from:
13/40/68/74/93/157, 13/40/68/157, and 40/68/81.

6. The engineered 3′O-kinase of claim 1, wherein the polypeptide sequence comprises one or more amino residue difference as compared to the reference sequence of SEQ ID NO: 450 at one or more amino acid positions selected from: 41/86/181/191, 41/46/190/191, 41/83/86/181/190/191, 41/181/191, 41/46/86/95/111, 41/46/86/95/191, 41/86/181/190/191, 41/86/181/191, 41/95/111/181/190/191, 41/190, 46/83/190/191, 46/86/181/190/191, 46/190/191, 48, 48/81, 48/81/103, 48/103/175/200, 48/103/200, 48/135, 48/135/175, 48/135/200, 48/200, 72, 72/82/88/124/166, 72/82/166, 72/82/91/124/166/182, 72/124/166, 72/166, 72/166/182, 72/182, 81, 81/103, 81/135, 81/135/200, 81/175/200, 81/200, 82, 82/88/91/124/166/182, 82/88/91/182, 82/88/124/166, 82/124/166, 82/124/166/182, 82/166/182, 86, 88/166, 91, 91/124/166/182/201, 91/166, 103, 103/135, 103/135/200, 103/175, 103/175/200, 103/200, 124, 124/166, 124/166/182, 135, 135/175/200, 135/200, 166, 166/182, 175, and 175/200.

7. The engineered 3′O-kinase of claim 1, wherein the polypeptide sequence comprises one or more amino residue difference as compared to the reference sequence of SEQ ID NO: 450 at one or more amino acid positions selected from: 2, 3, 4, 5, 6, 7, 13, 15, 17, 19, 22, 25, 26, 28, 29, 32, 35, 36, 44, 47, 49, 50, 51, 52, 53, 55, 56, 57, 58, 59, 60, 61, 63, 77, 78, 79, 84, 85, 92, 94, 97, 98, 100, 101, 104, 105, 109, 121, 122, 125, 126, 127, 129, 136, 139, 142, 149, 152, 153, 167, 170, 171, 173, 182, 191, 194, 195, 196, 197, 199, 200, 201, 202, 203, and 204.

8. The engineered 3′O-kinase of claim 1, wherein the polypeptide sequence comprises one or more amino residue difference as compared to the reference sequence of SEQ ID NO: 496 at one or more amino acid positions selected from: 3/49/105/124/125/200, 3/49/61/81/83/124/125/166/171, 3/49/61/81/124/125/200, 3/49/61/83, 3/49/61/83/125/166, 3/49/61/83/200, 3/49/61/124/125/171/200, 3/49/81/83/124/125/171, 3/49/81/83/124/125/200, 3/49/81/124/125/166/171, 3/49/124/125/166/171/200, 3/49/166/171, 3/61/81/105/124/125/166/171, 3/61/81/125/166/171/200, 3/81/105/124/166, 3/81/124/125/200, 3/83/166/171, 3/166/171, 49/61/81/83, 49/61/83/105/124/125/171, 49/61/83/124/125/171, 49/61/124/125/166/171, 49/61/125/171, 49/83/105/111/124, 49/83/105/124/125/166, 49/111/124/166/171/200, 49/124/125/166, 50/60/72/86/103, 50/60/82/83/103/126/142/175/191, 50/91/126/135, 60/182, 61/81/83/166/171/200, 61/81/125/166/171/200, 61/83/124/125/200, 61/124/125/166/171/200, 61/125, 61/166, 61/200, 72/82/83/142/181/191/200, 72/86/91/97/135, 72/142/182, 82/83, 82/83/103, 83/91/94/95/126/135/191, 83/105/124/125/166/200, 83/105/166, 83/125/171, 86/94/111/126/142, 86/126/135/142, 94/126, 105, 111/126/135/175/182, 124/125, 126, 142, 182, and 200.

9. The engineered 3′O-kinase of claim 1, wherein the polypeptide sequence comprises one or more amino residue difference as compared to the reference sequence of SEQ ID NO: 496 at one or more amino acid positions selected from: 3/49/61/83/200, 3/49/105/124/125/200, 72/82/83/142/181/191/200, 126, 142, and 191/200.

10. The engineered 3′O-kinase of claim 1, wherein the polypeptide sequence comprises one or more amino residue difference as compared to the reference sequence of SEQ ID NO: 1042 at one or more amino acid positions selected from: 53/100/105, 7, 7/28/32/97, 7/61, 7/61/85/97/126, 7/85/97/126, 13, 19, 19/53/105, 19/53/201, 19/100/105/201, 28/32/71/79/97/126/204, 28/32/85, 28/32/97/126, 28/36/61, 32/36/61/85/97/126/204, 32/36/126, 32/85/126/204, 35/50, 36/61/126/204, 50/78/142, 53/58/100/105/109, 53/58/109/201, 61, 79, 79/126/204, 85, 85/97, 85/126, 97, 100, 100/105, 105/201, 126, 171/201, and 204.

11. The engineered 3′O-kinase of claim 1, wherein the polypeptide sequence comprises one or more amino residue difference as compared to the reference sequence of SEQ ID NO: 1042 at one or more amino acid positions selected from: 10, 11, 20, 23, 27, 38, 39, 41, 62, 64, 67, 68, 69, 71, 71/131, 72, 74, 89, 93, 95, 96, 103, 110, 115, 117, 124, 130, 134, 135, 141, 146, 148, 150, 156, 157, 158, 160, 161, 163, 165, 166, 175, 176, 181, 182, and 192.

12. The engineered 3′O-kinase of claim 1, wherein the polypeptide sequence comprises one or more amino residue difference as compared to the reference sequence of SEQ ID NO: 1180 at one or more amino acid positions selected from: 127, 3/25/29/60/170, 3/25/29/126, 3/25/126, 3/44/126/170, 25/44/58, 25/58/60, 44/58/60/61/126/170, 58/61/126, 167/171/173, and 170.

13. The engineered 3′O-kinase of claim 1, wherein the polypeptide sequence comprises one or more amino residue difference as compared to the reference sequence of SEQ ID NO: 1180 at one or more amino acid positions selected from: 17/63, 17/63/104, 17/63/104/125, 22/55/98/127, 22/55/98/167/171/173/197, 25/29/60/126, 25/36/126, 28, 44/60/61/126, 59/104/125, 63/125, 79/125/129, 98/167/171, and 127.

14. The engineered 3′O-kinase of claim 1, wherein the polypeptide sequence comprises one or more amino residue difference as compared to the reference sequence of SEQ ID NO: 1412 at one or more amino acid positions selected from: 68/71/157/184, 27, 27/68, 27/68/71/184, 27/71, 27/71/184, 27/95, 41/72/160/161, 68/71/113, 68/71/113/157/176, 71, and 71/184.

15. The engineered 3′O-kinase of claim 1, wherein the polypeptide sequence comprises one or more amino residue difference as compared to the reference sequence of SEQ ID NO: 1412 at one or more amino acid positions selected from: 49, 52, 61, 83, and 125.

16. The engineered 3′O-kinase of claim 1, wherein the polypeptide sequence comprises one or more amino residue difference as compared to the reference sequence of SEQ ID NO: 1412 at one or more amino acid positions selected from: 2, 3, 4, 13, 28, 30, 32, 35, 36, 40, 43, 45, 48, 49, 51, 52, 53, 54, 56, 57, 60, 61, 63, 76, 77, 78, 80, 81, 82, 83, 85, 86, 90, 92, 94, 97, 100, 101, 104, 109, 121, 127, 129, 133, 139, 152, 153, 154, 167, 173, 186, 191, 194, 197, and 198.

17. The engineered 3′O-kinase of claim 1, wherein the polypeptide sequence comprises one or more amino residue difference as compared to the reference sequence of SEQ ID NO: 1412 at one or more amino acid positions selected from: 2, 19, 35, 45, 48, 49, 52, 53, 61, 76, 78, 80, 83, 85, 98, 106, 109, 121, 125, 170, 171, 194, and 195.

18. The engineered 3′O-kinase of claim 1, wherein the polypeptide sequence comprises one or more amino residue difference as compared to the reference sequence of SEQ ID NO: 1412 at one or more amino acid positions selected from: 28, 40, 48, 49, 51, 57, 60, 80, 82, 83, 92, 94, 98, 100, 104, 109, 127, 171, 186, 193, 194, 195, and 198.

19. The engineered 3′O-kinase of claim 1, wherein the polypeptide sequence comprises one or more amino residue difference as compared to the reference sequence of SEQ ID NO: 1464 at one or more amino acid positions selected from: 64/150/181, 3/20/74/103, 10/23/27/38/49/113, 10/27/38/49, 10/83, 23/27/49/83/125/141, 27/49/74, 27/60/83/125, 27/83/113, 39, 41/64/72/103/160, 41/64/103/117/150/160/161, 49/60, 49/64/96/113/175, 49/68/134, 60/61, 60/175, 61/110/146/151, 64, 64/72/115/150, 64/103/150/181, 64/161, 68/72/83/175, 72/103/124/160/161, 72/103/125/150/160/181, 72/124/150/160/181, 74/165, 103/182, 117/150, 150/160/181, 150/181, 151, 160/181, 182, and 192.

20. The engineered 3′O-kinase of claim 1, wherein the polypeptide sequence comprises one or more amino residue difference as compared to the reference sequence of SEQ ID NO: 1464 at one or more amino acid positions selected from: 20/103/192, 52/61, 61/110/165, 64/72/115/150, 72, 72/103/125/150/160/181, and 192.

21. The engineered 3′O-kinase of claim 1, wherein the polypeptide sequence comprises one or more amino residue difference as compared to the reference sequence of SEQ ID NO: 1464 at one or more amino acid positions selected from: 16, 20, 35, 68, 75, 85, 88, 89, 93, 122, 127, 134, 139, 146, 148, 150, 151, 161, 165, 182, and 182/205.

22. The engineered 3′O-kinase of claim 1, wherein the polypeptide sequence comprises one or more amino residue difference as compared to the reference sequence of SEQ ID NO: 1464 at one or more amino acid positions selected from: 7, 18, 20, 22, 35, 67, 68, 71, 75, 81, 85, 88, 89, 121, 136, 137, 139, 141, 142, 146, 148, 150, 151, 153, 160, 161, 176, 182, 182/205, and 185.

23. The engineered 3′O-kinase of claim 1, wherein the polypeptide sequence comprises one or more amino residue difference as compared to the reference sequence of SEQ ID NO: 1464 at one or more amino acid positions selected from: 18, 20, 23, 29, 30, 35, 36, 37, 38, 40, 71, 85, 89, 93, 95, 113, 127, 142, 146, 161, 165, and 185.

24. The engineered 3′O-kinase of claim 1, wherein the polypeptide sequence comprises one or more amino residue difference as compared to the reference sequence of SEQ ID NO: 1464 at one or more amino acid positions selected from: 8, 11, 15, 88, 113, 133, 143, 155, and 161.

25. The engineered 3′O-kinase of claim 1, wherein the polypeptide sequence comprises one or more amino residue difference as compared to the reference sequence of SEQ ID NO: 1800 at one or more amino acid positions selected from: 60/61, 27, 27/49/51, 27/49/171, 40, 40/92/104, 48, 48/53/60/76/80/193, 48/56/60/76/167/170/193, 49, 53/56/60/76, 56/60, 56/60/76/78/80, 56/60/85/193, 56/76/80/170, 56/76/80/193, 56/85/104, 56/167/193, 60, 60/193, 76/80, 98, 101, 101/109/198, 125, 165, 171/186, and 186.

26. The engineered 3′O-kinase of claim 1, wherein the polypeptide sequence comprises one or more amino residue difference as compared to the reference sequence of SEQ ID NO: 2078 at one or more amino acid positions selected from: 48, 52, 100, 165, and 193.

27. The engineered 3′O-kinase of claim 1, wherein 3′O-kinase comprises a polypeptide sequence having at least 85%, 90%, 91%, 92%, 93%, 94%, 95%, 96%, 97%, 98%, 99%, or more sequence identity to an even-numbered sequence selected from SEQ ID NOs: 56-366, or 372-2122.

28. The engineered 3′O-kinase of claim 1, wherein said engineered 3′O-kinase has activity in the conversion of a natural or modified NTP to a nucleoside triphosphate with an additional phosphate at the 3′ position of the sugar.

29. The engineered 3′O-kinase of claim 1, comprising at least one improved property, as compared to a wild-type or reference 3′O-kinase, wherein said improved Property comprises increased activity, increased activity on non-natural substrates, increased selectivity, increased substrate Promiscuity, decreased product inhibition and/or decreased byproduct formation, as compared to a wild-type or reference 3′O-kinase.

30. (canceled)

31. The engineered 3′O-kinase of claim 1, wherein said 3′O-kinase comprises increased selectivity toward the nucleoside tetraphosphate (NQP) over 4 pA or other byproduct species, as compared to a wild-type or reference 3′O-kinase.

32. The engineered 3′O-kinase of claim 1, wherein said 3′O-kinase comprises increased activity in the conversion of a natural or modified NTP to a nucleoside triphosphate with an additional phosphate at the 3′ position of the sugar, as compared to a wild-type or reference 3′O-kinase.

33. The engineered 3′O-kinase of claim 1, wherein said 3′O-kinase is purified.

34. A polynucleotide encoding at least one engineered 3′O-kinase of claim 1.

35-39. (canceled)

40. An expression vector comprising at least one polynucleotide claim 34.

41. A host cell comprising at least one expression vector of claim 40.

42. A method of producing an engineered 3′O-kinase polypeptide in a host cell comprising culturing a host cell of claim 41, under suitable culture conditions, such that at least one engineered 3′O-kinase is produced.

43. (canceled)

44. (canceled)

45. A composition comprising at least one engineered 3′O-kinase of claim 1.

46. A method of producing an NTP with a phosphate group at the 3′ position of the sugar (NQP), the method comprising (i) providing a 3′O-kinase enzyme, and (ii) contacting the 3′O-kinase enzyme with an NTP under suitable reaction conditions, such that an NQP is produced.

47. (canceled)

48. (canceled)

49. The method of claim 46, further comprising a modification of the NTP and/or NQP at the 2′ position of the sugar.

50-52. (canceled)

53. The method of claim 46, further comprising a modification of the NTP and/or NQP at the phosphate chain.

54. The method of claim 53, wherein the modification of the NTP and/or NQP at the phosphate chain comprises an α-phosphothioate linkage.

55. The method of claim 46, further comprising a phosphate donor.

56. The method of claim 55, wherein the phosphate donor comprises acetyl phosphate, polyphosphate, or an NTP.

57. The method of claim 55, further comprising a phosphate donor that is the same or is a different type of NTP than the substrate NTP.

58. The method of claim 46, further comprising a phosphate recycling system.

59. (canceled)

60. The method of claim 58, wherein the phosphate recycling system comprises a phosphate donor and a kinase, wherein the kinase comprises acetate kinase or polyphosphate kinase/transferase.

61. The method of claim 58, further comprising a pyruvate oxidase enzyme.

62. (canceled)

63. (canceled)

64. The method of any claim 46, wherein the 3′O-kinase comprises an engineered 3′O-kinase of claim 1.

65. (canceled)

66. The method of claim 64, wherein said engineered 3′O-kinase converts an NTP to an NQP with a conversion rate that is at least 1.5 fold, 2 fold, 5 fold, 10 fold or more increased, as compared to a wild type or reference 3′O-kinase.

67. The method of claim 64, wherein the engineered 3′O-kinase has increased activity, increased activity on non-natural substrates, increased selectivity, increased substrate promiscuity, decreased product inhibition and/or decreased byproduct formation, as compared to a wild-type or reference 3′O-kinase known to those of skill in the art.

68-111. (canceled)

112. The engineered 3′O-kinase of claim 1, wherein said engineered 3′O-kinase is immobilized.

113-115. (canceled)
